(19) United States
(12) Patent Application Publication
(10) Pub. No.: US 2024/0327522 A1
(43) Pub. Date: Oct. 3, 2024

(54) ANTI-ALPHA V BETA 8 INTEGRIN ANTIBODIES AND METHODS OF USE

(71) Applicant: Genentech, Inc., South San Francisco, CA (US)

(72) Inventors: WeiYu LIN, Millbrae, CA (US); Kelly Michele LOYET, San Mateo, CA (US); Matthieu MASUREEL, San Carlos, CA (US); Minh THAI, Alameda, CA (US); Shannon Jennifer TURLEY, Cambridge, MA (US); Tulika TYAGI, San Mateo, CA (US); Yan WU, Foster City, CA (US); Jianhuan ZHANG, Union City, CA (US); Alessandra CASTIGLIONI, San Carlos, CA (US); Cecilia Pui Chi CHIU, Redwood City, CA (US)

(73) Assignee: Genentech, Inc., South San Francisco, CA (US)

(21) Appl. No.: 18/621,505

(22) Filed: Mar. 29, 2024

Related U.S. Application Data
(60) Provisional application No. 63/536,342, filed on Sep. 1, 2023.
(60) Provisional application No. 63/456,246, filed on Mar. 31, 2023.

(51) Int. Cl. C07K 16/28; A61K 39/00; A61P 35/00

(52) U.S. Cl. CPC C07K 16/2842; A61P 35/00; C07K 16/2818; A61K 2039/507; C07K 2317/24; C07K 2317/52; C07K 2317/565; C07K 2317/76; C07K 2317/92

(57) ABSTRACT

Provided herein are anti-αvβ8 antibodies or antigen-binding portions thereof and methods of making and using the same. Further provided are methods of treating cancer with an anti-αvβ8 and a PD-1 axis antagonist.

### CROSS-REFERENCE TO RELATED APPLICATIONS

This application claims priority to U.S. Provisional Patent Application No. 63/456,246, filed Mar. 31, 2023, and U.S. Provisional Patent Application No. 63/536,342, filed Sep. 1, 2023, each entitled “ANTI-ALPHA V BETA 8 INTEGRIN ANTIBODIES AND METHODS OF USE”, the contents of each of which are hereby incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 146392064800SeqList.xml, created Mar. 25, 2024, which is 157,697 bytes in size. The information in the electronic format of the Sequence Listing is herein incorporated by reference in its entirety.

### TECHNICAL FIELD

The present invention relates to anti-αvβ8 antibodies and methods of using the same.

### BACKGROUND

The transforming growth factor β (TGFβ) exists in an inactive dormant complex of which most cells produce the cytokine and/or express receptors for it. TGFβ acts locally and is highly dependent on the cellular context and the tissue microenvironment. TGFβ can promote cancer progression through highly regulated and differential effects on multiple cells type in the microenvironment. It is also associated with reduced survival of cancer patients and lack of response to checkpoint inhibitors and other anti-cancer drugs.

TGFβ complexes must be activated to release active cytokine. Certain integrins, such as αvβ8, and proteases convert latent TGFβ into active cytokine. Antibodies which bind to integrin αvβ8 and reduce ligand binding function are needed.

### SUMMARY

The invention provides anti-αvβ8 antibodies and methods of using the same.

Provided herein is an antibody, or antigen-binding portion thereof, that specifically binds to αvβ8, wherein the antibody or antigen-binding portion thereof exhibits at least one of the following properties: (a) binds to human αvβ8 with a KD of 1 nM or less; (b) binds to murine αvβ8 with a KD of 1 nM or less; (c) binds to cynomolgus αvβ8 with a KD of 1 nM or less; (d) inhibits αvβ8-mediated activation of LTGFβ 1 and LTGFβ3 presented by and/or associated with human-leucine-rich-repeat-containing protein 32 (LRRC32), LRRC32, and/or latent TGFβ-binding proteins (LTBPs); and/or (e) blocks binding of TGFβ peptide to αvβ8. In some embodiments, the antibody, or antigen-binding portion thereof, comprises a light chain variable domain (VL) comprising CDR-L1, CDR-L2, and CDR-L3 and a heavy chain variable domain (VH) comprising CDR-H1, CDR-H2, and CDR-H3, wherein: (a) CDR-L1, CDR-L2, and CDR-L2 sequences are from a VL domain of SEQ ID NO:152 and CDR-H1, CDR-H2, and CDR-H3 sequences are from a VH domain of SEQ ID NO:153; (b) CDR-L1, CDR-L2, and CDR-L2 sequences are from a VL domain of SEQ ID NO:154 and CDR-H1, CDR-H2, and CDR-H3 sequences are from a VH domain of SEQ ID NO:155; (c) CDR-L1, CDR-L2, and CDR-L2 sequences are from a VL domain of SEQ ID NO:156 and CDR-H1, CDR-H2, and CDR-H3 sequences are from a VH domain of SEQ ID NO:157; (d) CDR-L1, CDR-L2, and CDR-L2 sequences are from a VL domain of SEQ ID NO:158 and CDR-H1, CDR-H2, and CDR-H3 sequences are from a VH domain of SEQ ID NO:159; (e) CDR-L1, CDR-L2, and CDR-L2 sequences are from a VL domain of SEQ ID NO:160 and CDR-H1, CDR-H2, and CDR-H3 sequences are from a VH domain of SEQ ID NO:161; (f) CDR-L1, CDR-L2, and CDR-L2 sequences are from a VL domain of SEQ ID NO:164 and CDR-H1, CDR-H2, and CDR-H3 sequences are from a VH domain of SEQ ID NO:165; (g) CDR-L1, CDR-L2, and CDR-L2 sequences are from a VL domain of SEQ ID NO:166 and CDR-H1, CDR-H2, and CDR-H3 sequences are from a VH domain of SEQ ID NO:167; (h) CDR-L1 is according to SEQ ID NO:7, CDR-L2 is according to SEQ ID NO:8, CDR-L3 is according SEQ ID NO:9, CDR-H1 is according to SEQ ID NO:10, CDR-H2 is according to SEQ ID NO:11, and CDR-H3 is according to SEQ ID NO:12; (i) CDR-L1 is according to SEQ ID NO:13, CDR-L2 is according to SEQ ID NO:14, CDR-L3 is according SEQ ID NO:15, CDR-H1 is according to SEQ ID NO:16, CDR-H2 is according to SEQ ID NO:17, and CDR-H3 is according to SEQ ID NO:18; (j) CDR-L1 is according to SEQ ID NO:19, CDR-L2 is according to SEQ ID NO:20, CDR-L3 is according SEQ ID NO:21, CDR-H1 is according to SEQ ID NO:22, CDR-H2 is according to SEQ ID NO:23, and CDR-H3 is according to SEQ ID NO:24; (k) CDR-L1 is according to SEQ ID NO:25, CDR-L2 is according to SEQ ID NO:26, CDR-L3 is according SEQ ID NO:27, CDR-H1 is according to SEQ ID NO:28, CDR-H2 is according to SEQ ID NO:29, and CDR-H3 is according to SEQ ID NO:30; (l) CDR-L1 is according to SEQ ID NO:31, CDR-L2 is according to SEQ ID NO:32, CDR-L3 is according SEQ ID NO:33, CDR-H1 is according to SEQ ID NO:34, CDR-H2 is according to SEQ ID NO:35, and CDR-H3 is according to SEQ ID NO:36; (m) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42; (n) CDR-L1, CDR-L2, and CDR-L2 sequences are from a VL domain of SEQ ID NO:150 and CDR-H1, CDR-H2, and CDR-H3 sequences are from a VH domain of SEQ ID NO:151; (o) CDR-L1 is according to SEQ ID NO:1, CDR-L2 is according to SEQ ID NO:2, CDR-L3 is according SEQ ID NO:3, CDR-H1 is according to SEQ ID NO:4, CDR-H2 is according to SEQ ID NO:5, and CDR-H3 is according to SEQ ID NO:6; (p) CDR-L1, CDR-L2, and CDR-L2 sequences are from a VL domain of SEQ ID NO:162 and CDR-H1, CDR-H2, and CDR-H3 sequences are from a VH domain of SEQ ID NO:163; or (q) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42.

Further provided herein is an antibody, or antigen-binding portion thereof, that specifically binds to αvβ8, wherein the antibody, or antigen-binding portion thereof, comprises a heavy chain variable domain (VH) comprising CDR-H1, CDR-H2, and CDR-H3 and a light chain variable domain (VL) comprising CDR-L1, CDR-L2, and CDR-L3, wherein: (a) CDR-L1, CDR-L2, and CDR-L2 sequences are from a VL domain of SEQ ID NO:152 and CDR-H1, CDR-H2, and CDR-H3 sequences are from a VH domain of SEQ ID NO:153; (b) CDR-L1, CDR-L2, and CDR-L2 sequences are from a VL domain of SEQ ID NO:154 and CDR-H1, CDR-H2, and CDR-H3 sequences are from a VH domain of SEQ ID NO:155; (c) CDR-L1, CDR-L2, and CDR-L2 sequences are from a VL domain of SEQ ID NO:156 and CDR-H1, CDR-H2, and CDR-H3 sequences are from a VH domain of SEQ ID NO:157; (d) CDR-L1, CDR-L2, and CDR-L2 sequences are from a VL domain of SEQ ID NO:158 and CDR-H1, CDR-H2, and CDR-H3 sequences are from a VH domain of SEQ ID NO:159; (e) CDR-L1, CDR-L2, and CDR-L2 sequences are from a VL domain of SEQ ID NO:160 and CDR-H1, CDR-H2, and CDR-H3 sequences are from a VH domain of SEQ ID NO:161; (f) CDR-L1, CDR-L2, and CDR-L2 sequences are from a VL domain of SEQ ID NO:164 and CDR-H1, CDR-H2, and CDR-H3 sequences are from a VH domain of SEQ ID NO:165; (g) CDR-L1, CDR-L2, and CDR-L2 sequences are from a VL domain of SEQ ID NO:166 and CDR-H1, CDR-H2, and CDR-H3 sequences are from a VH domain of SEQ ID NO:167; (h) CDR-L1 is according to SEQ ID NO:7, CDR-L2 is according to SEQ ID NO:8, CDR-L3 is according SEQ ID NO:9, CDR-H1 is according to SEQ ID NO:10, CDR-H2 is according to SEQ ID NO:11, and CDR-H3 is according to SEQ ID NO:12; (i) CDR-L1 is according to SEQ ID NO:13, CDR-L2 is according to SEQ ID NO:14, CDR-L3 is according SEQ ID NO:15, CDR-H1 is according to SEQ ID NO:16, CDR-H2 is according to SEQ ID NO:17, and CDR-H3 is according to SEQ ID NO:18; (j) CDR-L1 is according to SEQ ID NO:19, CDR-L2 is according to SEQ ID NO:20, CDR-L3 is according SEQ ID NO:21, CDR-H1 is according to SEQ ID NO:22, CDR-H2 is according to SEQ ID NO:23, and CDR-H3 is according to SEQ ID NO:24; (k) CDR-L1 is according to SEQ ID NO:25, CDR-L2 is according to SEQ ID NO:26, CDR-L3 is according SEQ ID NO:27, CDR-H1 is according to SEQ ID NO:28, CDR-H2 is according to SEQ ID NO:29, and CDR-H3 is according to SEQ ID NO:30; (l) CDR-L1 is according to SEQ ID NO:31, CDR-L2 is according to SEQ ID NO:32, CDR-L3 is according SEQ ID NO:33, CDR-H1 is according to SEQ ID NO:34, CDR-H2 is according to SEQ ID NO:35, and CDR-H3 is according to SEQ ID NO:36; (m) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42; (n) CDR-L1, CDR-L2, and CDR-L2 sequences are from a VL domain of SEQ ID NO:150 and CDR-H1, CDR-H2, and CDR-H3 sequences are from a VH domain of SEQ ID NO:151; (o) CDR-L1 is according to SEQ ID NO:1, CDR-L2 is according to SEQ ID NO:2, CDR-L3 is according SEQ ID NO:3, CDR-H1 is according to SEQ ID NO:4, CDR-H2 is according to SEQ ID NO:5, and CDR-H3 is according to SEQ ID NO:6; (p) CDR-L1, CDR-L2, and CDR-L2 sequences are from a VL domain of SEQ ID NO:162 and CDR-H1, CDR-H2, and CDR-H3 sequences are from a VH domain of SEQ ID NO:163; or (q) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42.

In some embodiments, the antibody, or antigen-binding portion thereof, is a monoclonal antibody. In some embodiments, the antibody, or antigen-binding portion thereof, is a humanized or chimeric antibody. In some embodiments, the antibody, or antigen-binding portion thereof, is an antibody fragment that specifically binds human αvβ8. In some embodiments, the antibody, or antigen-binding portion thereof, comprises a sequence selected from the group consisting of (a) a VL sequence having at least 95% sequence identity to the amino acid sequence of SEQ ID NO:152 and a VH sequence having at least 95% sequence identity to the amino acid sequence of SEQ ID NO:153; (b) a VL sequence having at least 95% sequence identity to the amino acid sequence of SEQ ID NO:154 and a VH sequence having at least 95% sequence identity to the amino acid sequence of SEQ ID NO:155; (c) a VL sequence having at least 95% sequence identity to the amino acid sequence of SEQ ID NO:156 and a VH sequence having at least 95% sequence identity to the amino acid sequence of SEQ ID NO:157; (d) a VL sequence having at least 95% sequence identity to the amino acid sequence of SEQ ID NO:158 and a VH sequence having at least 95% sequence identity to the amino acid sequence of SEQ ID NO:159; (e) a VL sequence having at least 95% sequence identity to the amino acid sequence of SEQ ID NO:160 and a VH sequence having at least 95% sequence identity to the amino acid sequence of SEQ ID NO:161; (f) a VL sequence having at least 95% sequence identity to the amino acid sequence of SEQ ID NO:164 and a VH sequence having at least 95% sequence identity to the amino acid sequence of SEQ ID NO:165; (g) a VL sequence having at least 95% sequence identity to the amino acid sequence of SEQ ID NO:166 and a VH sequence having at least 95% sequence identity to the amino acid sequence of SEQ ID NO:167; and (h) a VL sequence having at least 95% sequence identity to the amino acid sequence of SEQ ID NO:162 and a VH sequence having at least 95% sequence identity to the amino acid sequence of SEQ ID NO:163.

In some embodiments, the antibody, or antigen-binding portion thereof, comprises a sequence selected from the group consisting of: (a) a VL sequence comprising the sequence of amino acids set forth in SEQ ID NO:152 and a VH sequence comprising the sequence of amino acids set forth in SEQ ID NO:153; (b) a VL sequence comprising the sequence of amino acids set forth in SEQ ID NO:154 and a VH sequence comprising the sequence of amino acids set forth in SEQ ID NO:155; (c) a VL sequence comprising the sequence of amino acids set forth in SEQ ID NO:156 and a VH sequence comprising the sequence of amino acids set forth in SEQ ID NO:157; (d) a VL sequence comprising the sequence of amino acids set forth in SEQ ID NO:158 and a VH sequence comprising the sequence of amino acids set forth in SEQ ID NO:159; (e) a VL sequence comprising the sequence of amino acids set forth in SEQ ID NO:160 and a VH sequence comprising the sequence of amino acids set forth in SEQ ID NO:161; (f) a VL sequence comprising the sequence of amino acids set forth in SEQ ID NO:164 and a VH sequence comprising the sequence of amino acids set forth in SEQ ID NO:165; (g) a VL sequence comprising the sequence of amino acids set forth in SEQ ID NO:166 and a VH sequence comprising the sequence of amino acids set forth in SEQ ID NO:167; (h) a VL sequence comprising the sequence of amino acids set forth in SEQ ID NO:150 and a VH sequence comprising the sequence of amino acids set forth in SEQ ID NO:151; and (i) a VL sequence comprising the sequence of amino acids set forth in SEQ ID NO:162 and a VH sequence comprising the sequence of amino acids set forth in SEQ ID NO:163.

In some embodiments, the antibody, or antigen-binding portion thereof, is a full-length antibody of IgG1 isotype. In some embodiments, the antibody, or antigen-binding portion thereof, comprises a variant IgG1 Fc region with reduced effector function. In some embodiments, the Fc region comprises amino acid substitutions L234A/L235A with numbering according to the EU index of Kabat. In some embodiments, the Fc region comprises amino acid substitution P329G with numbering according to the EU index of Kabat. In some embodiments, the antibody binds human αvβ8 with a KD of 1 nM or less as measured by surface plasmon resonance. In some embodiments, the antibody, or antigen-binding portion thereof, comprises: (a) a heavy chain exhibiting at least 95% sequence identity to the sequence of amino acids set forth in SEQ ID NO:201 and a light chain exhibiting at least 95% sequence identity to the sequence of amino acids set forth in SEQ ID NO:200; or (b) a heavy chain exhibiting at least 95% sequence identity to the sequence of amino acids set forth in SEQ ID NO:203 and a light chain exhibiting at least 95% sequence identity to the sequence of amino acids set forth in SEQ ID NO:202. In some embodiments, the antibody, or antigen-binding portion thereof, comprises: (a) a heavy chain exhibiting at least 95% sequence identity to the sequence of amino acids set forth in SEQ ID NO:201 and a light chain exhibiting at least 95% sequence identity to the sequence of amino acids set forth in SEQ ID NO:200; or (b) a heavy chain exhibiting at least 95% sequence identity to the sequence of amino acids set forth in SEQ ID NO:221 and a light chain exhibiting at least 95% sequence identity to the sequence of amino acids set forth in SEQ ID NO:202. In some embodiments, the antibody, or antigen-binding portion thereof, comprises: (a) a heavy chain exhibiting at least 95% sequence identity to the sequence of amino acids set forth in SEQ ID NO:220 and a light chain exhibiting at least 95% sequence identity to the sequence of amino acids set forth in SEQ ID NO:200; or (b) a heavy chain exhibiting at least 95% sequence identity to the sequence of amino acids set forth in SEQ ID NO:203 and a light chain exhibiting at least 95% sequence identity to the sequence of amino acids set forth in SEQ ID NO:202. In some embodiments, the antibody, or antigen-binding portion thereof, comprises: (a) a heavy chain of SEQ ID NO:201 and a light chain of SEQ ID NO:200; or (b) a heavy chain of SEQ ID NO:203 and a light chain of SEQ ID NO:202. In some embodiments, the antibody, or antigen-binding portion thereof, comprises: (a) a heavy chain of SEQ ID NO:220 and a light chain of SEQ ID NO:200; or (b) a heavy chain of SEQ ID NO:221 and a light chain of SEQ ID NO:202.

Further provided herein is an isolated nucleic acid encoding any one of the preceding antibodies. Further provided herein is a vector comprising the nucleic acid. Further provided herein is a host cell comprising the nucleic acid or the vector. Further provided herein is a method of producing an antibody that binds to αvβ8 comprising culturing the host cell under conditions suitable for the expression of the antibody. In some embodiments, the method further comprises recovering the antibody from the host cell. Further provided herein is an antibody produced by the method.

Further provided herein is a pharmaceutical composition comprising any of the preceding antibodies, or an antigen-binding portion thereof, and a pharmaceutically acceptable carrier.

Further provided herein is a method of treating cancer in an individual in need thereof, the method comprising administering to the individual an effective amount of one of the preceding antibodies, or an antigen-binding portion thereof, or the preceding pharmaceutical composition.

Further provided herein is a method of treating cancer in an individual in need thereof, the method comprising administering: (a) an effective amount of one of the preceding antibodies or an antigen-binding fragment thereof or the preceding pharmaceutical composition, and (b) an effective amount of a PD-1 axis antagonist. In some embodiments, the PD-1 axis antagonist is an anti-PD-L1 antibody. In some embodiments, the PD-1 axis antagonist is atezolizumab. In some embodiments, the method further comprises assessing the level of αvβ6 expressed in the cancer.

In some embodiments of the preceding methods, the cancer is selected from the list of ovarian cancer, triple-negative breast cancer, non-small cell lung cancer, colorectal cancer, cholangiocarcinoma, endometrial cancer, kidney renal papillary cancer, and bladder cancer.

### BRIEF DESCRIPTION OF THE FIGURES

Representative embodiments of the invention are disclosed by reference to the following figures. It should be understood that the embodiments depicted are not limited to the precise details shown.

FIG. 1 shows a schematic of a screening method for αVβ8 antibody.

FIGS. 2A and 2B show IC50 for rabbit αvβ8 antibodies compared to C6D4 mIgG2a LALALG control.

FIGS. 3A-3C show binding plots for rabbit αvβ8 antibodies (rb.avb8-65, FIG. 3A, and rb.avb8-92, FIG. 3B) compared to a control antibody (hu.C6D4, FIG. 3C) for cynomolgus (cyno), human (hu) or murine (mu) αvβ8. FIG. 3D shows binding plots for rabbit αvβ8 antibodies (rb.avb8-65, middle, and rb.avb8-92, right) in the presence of two metal ions (e.g., divalent cations) compared to a control antibody (C6D4).

FIGS. 4A and 4B show sequence alignment of rabbit and humanized αvβ8 antibodies. CDR sequences are underlined and identified according to Kabat numbering system. FIG. 4A shows sequence alignment of the light chain variable region. FIG. 4B shows sequence alignment of the heavy chain variable region.

FIGS. 5A-5D demonstrate CryoEM results highlighting the interaction between rb.αvβ8-65 (anti-αvβ8 integrin antibody) and αvβ8 integrin (FIG. 5A and FIG. 5B (rotated 90° relative to FIG. 5A)), latent-TGFβ1 (L-TGFβ1) and αvβ8 integrin (FIG. 5C and FIG. 5D (rotated 90° relative to FIG. 5A)). FIGS. 5A-5D demonstrate that the binding of Fab65 blocks the interaction of latent-TGFβ1 to αvβ8. FIGS. 5C and 5D shown the position of the RGDLXXI/L motif of L-TGFb1 slotting into the interface between αV and β8 subunits. FIGS. 5A and 5B demonstrate that the FAB65 occupies a similar position as L-TGFb1 with respect to αvβ8, effectively blocking the L-TGFb1 binding site.

FIGS. 5E-5H are expanded views of the interface between rb.αvβ8-65 (anti-αvβ8 integrin antibody) and αvβ8 integrin (FIGS. 5E and 5G) and the interface between L-TGFβ1 and αvβ8 integrin (FIGS. 5F and 5H). Several residues from αvβ8 integrin that interact with either rb.αvβ8-65 or L-TGFβ1 are highlighted, respectively. FIGS. 5E and 5G demonstrate that residues F177 and D218 of αV make specific contacts with CDRH2; K119, Q120, E121 and D148 of αV make specific contacts with CDRL1; N219 of β8 makes specific contacts with CDRH2; and R164 of 38 makes specific contacts with CDRL1.

FIGS. 5I-5K are expanded views of the interfaces between rb.αvβ8-65 (anti-αvβ8 integrin antibody) and αvβ8 integrin showing salt bridges formed between the hu.αvβ8-65 (anti-αvβ8 integrin antibody) and αvβ8 integrin. Particular residues are highlighted.

FIG. 5L shows the sequence of the αV and β8 subunits of the αvβ8 and an EM structure of the interface between rb.αvβ8-65 (anti-αvβ8 integrin antibody) and αvβ8 integrin. Residues within 5 Å of rb.αvβ8-65 are highlighted in the sequence of the αV (i.e., R115, 118M, 119K, 120Q, 121E, 123E, 1471, 148D, 149A, 150D, 154F, 177F, 178Y, 180Q, 212T, 213A, 214Q, 215A, and 218D) and β8 (i.e., 118H, 119N, 122E, 1581, 159S, 1601, 164R, 166H, 169C, 170S, 171D, 172Y, 206G, 207N, 2081) subunits of the αvβ8 and depicts the putative αvβ8 epitope as bound by Fab65.

FIG. 5M demonstrates subtype specificity assessment of binding between Fab65 and αV compared to other αV integrins. The alignment is based on low sequence conservation of αV epitope. Fab65 is specific to αV and should not bind to other a integrins.

FIG. 5N demonstrates subtype specificity assessment of binding between Fab65 and β8 compared to other β8 integrins. The alignment is based on low sequence conservation of 38 epitope. Fab65 is specific to β8 and should not bind to other 3 integrins. Backbone of Fab65 CDRL3 Gly95a forms hydrogen bond with backbone of αvβ8 Ser159 (Lys in αvβ6. Counter-screening against αvβ1, αvβ3, αvβ5, and αvβ6 was done during antibody discovery.

FIGS. 6A and 6B show binding plots for two separate experiments for humanized αvβ8 antibodies (rb.aVb8-65) binding to cynomolgus (cyno), human (hu) or murine (mu) antigen.

FIGS. 7A and 7B show binding plots for two separate experiments for humanized αvβ8 antibodies (rb.aVb8-92) binding to cynomolgus (cyno), human (hu) or murine (mu) antigen.

FIG. 8 shows surface plasmon sensograms for the binding of anti-αvβ8-65 to recombinant αvβ8 proteins from human, cynomolgus monkey, rat, and mouse. The recombinant αvβ8 concentrations for each sensogram (from bottom to top) were 3.7 nM, 11.1 nM, 33.3 nM, and 100 nM. Black solid lines are fitted curves using a 1:1 binding model, and colored lines represent actual data.

FIGS. 9A and 9B show surface plasmon resonance sensograms for binding of αvβ8 to hC6D4 and hADWA11. FIG. 9A shows binding of hC6D4 (left) and αvβ8-65 (right) to human αvβ8 protein. Black solid lines are fitted curves using a 1:1 binding model, and colored lines are the actual data. The recombinant αvβ8 concentrations (from bottom to top) are 0.8, 4, 20, and 100 nM. Format: Protein A capture, 37° C., 100 μl/min, HBS-P pH7.2, 0.5 mM CaCl₂). FIG. 9B shows surface plasmon resonance sensograms for the binding of αvβ8-65 (R07566802; left) and hADWA11-2.4 (right) to human αvβ8 protein. Black solid lines are fitted curves using a 1:1 binding model, and colored lines are the actual data. The recombinant αvβ8 concentrations (from bottom to top) are 3.7, 11.1, 33.3, and 100 nM. Format: Protein A capture, 37° C., 100 μl/min, HBS-P pH7.2, 0.5 mM CaCl₂).

FIG. 10 shows surface plasmon resonance sensograms for the binding of αvβ8-65 (R07566802; left), hADWA11-2.4 (middle) and hC6D4 (right) to human αvβ8 protein. Black solid lines are fitted curves using a 1:1 binding model, and colored lines are the actual data. The recombinant αvβ8 concentrations (from bottom to top) are 0.4, 2, 10, and 50 nM. Format: Anti-murine Fc capture, 37° C., 100 μl/min, HBS-P pH7.2, 0.5 mM CaCl₂).

FIGS. 11A-11D show four experiments depicting the ability of αvβ8 antibody to block binding of αvβ8 to TGFβ1 or TGFβ3. FIG. 11A shows experiment 1, which is a co-culture assay of LN-229 and 3T3-Nano-human GARP/LTGFb1 (WT). FIG. 11B shows experiment 2, which is a co-culture assay of LN-229 and 3T3-Nano-human GARP/LTGFb1 (WT). FIG. 11C shows experiment 3, which is a co-culture assay of LN-229 and 3T3-Nano-human GARP/LTGFb1 (non-releasable (NR)). FIG. 11D shows experiment 4, which is a co-culture assay of LN-229 and 3T3-Nano-human GARP/LTGFb3 (WT).

FIGS. 12A-12H show competition assays between αvβ8 and ADWA11 or C6D4 in EMT6 cells or HCC1159 cells. FIG. 12A shows a competition assay between αvβ8 and ADWA11 (1 μg/mL) in the EMT6 cell line. FIG. 12B shows a competition assay between αvβ8 and ADWA11 (40 μg/mL) in the EMT6 cell line. FIG. 12C shows a competition assay between αvβ8 and C6D4 (1 μg/mL) in the EMT6 cell line. FIG. 12D shows a competition assay between αvβ8 and C6D4 (40 μg/mL) in the EMT6 cell line. FIG. 12E shows a competition assay between αvβ8 and ADWA11 (1 μg/mL) in the HCC1159 cell line. FIG. 12F shows a competition assay between αvβ8 and ADWA11 (10 μg/mL) in the HCC1159 cell line. FIG. 12G shows a competition assay between αvβ8 and C6D4 (1 μg/mL) in the HCC1159 cell line. FIG. 12H shows a competition assay between αvβ8 and C6D4 (10 μg/mL) in the HCC1159 cell line.

FIG. 13 shows pharmacokinetics of humanized αvβ8 antibodies in SCID mouse.

FIG. 14 shows pharmacokinetics of humanized αvβ8 antibodies in cynomolgus monkey.

FIG. 15 shows pharmacokinetics of humanized αvβ8 antibodies at two doses in CD-1 mouse.

FIGS. 16A and 16B show the percentage of pSMAD/SMAD in tumor and lymph node (LN).

FIG. 17 shows tumor volume in mice receiving one or two antibodies. GP120 antibody serves as a negative control. aPDL1 antibody (mu.C6D4) was administered alone or in combination with rb.aVb8-65 (c65), or rb.aVb8-92 (c92). Complete response (CR) is shown as a percentage.

FIGS. 18A and 18B show tumor volume in mice of the EMT6 mouse model receiving one or two antibodies. GP120 antibody serves as a negative control. aPDL1 antibody (mu.C6D4) was administered alone or in combination with hu.ADWA11, rb.aVb8-65 (c65), hu.aVb8-65, or hu.aVb8-92 (c92). Complete response (CR) is shown as a percentage.

FIGS. 19A and 19B show the calculation of complete response (CR) in anti-PD-L1 in combination with ADWA11 compared to anti-PD-L1 in combination with αvβ8-65. FIG. 19A shows % of CR in anti-PD-L1+ADWA11 compared to anti-PD-L1+αvβ8-65. FIG. 19B shows % of CR in anti-PD-L1+ADWA11 compared to anti-PD-L1+αvβ8-65, direct comparison. Each dot represents one study with 10 mice per group of treatment.

FIGS. 20A and 20B show tumor volume in mice of the MC38 mouse model receiving one or two antibodies. GP120 antibody serves as a negative control. aPDL1 antibody (mu.C6D4) was administered alone or in combination with hu.ADWA11, rb.aVb8-65 (c65), hu.αVB8-65, hu.aVb8-92. Complete response (CR) is shown as a percentage.

FIG. 21 shows the anti-tumor activity of Ch-anti-αvβ8-65 and anti-αvβ8-ADWA11, each in combination with anti-PD-L1, compared to isotype control and anti-PD-L1 alone.

FIGS. 22A, 22B, and 22C show comparison of anti-tumor activity of Ch-anti-αvβ8-65 and anti-αvβ8-ADWA11 by (FIG. 22A) percentage of CRs in the combination groups compared to anti-PD-L1 alone across several studies, (FIG. 22B) percentage of CRs in the combination groups compared to anti-PD-L1 alone in direct comparison (i.e., the two molecules were used within the same study), and (FIG. 22C) the percentage of mice with regressing tumors at day 14, measured in studies in which both molecules were tested.

### DETAILED DESCRIPTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The terms “anti-αvβ8 antibody” and “an antibody that binds to αvβ8” refer to an antibody that is capable of binding αvβ8 with sufficient affinity such that the antibody is useful as a diagnostic and/or therapeutic agent in targeting αvβ8. In one aspect, the extent of binding of an anti-αvβ8 antibody to an unrelated, non-αvβ8 protein is less than about 10% of the binding of the antibody to αvβ8 as measured, e.g., by surface plasmon resonance (SPR). In certain aspects, an antibody that binds to αvβ8 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to αvβ8 when the antibody has a K_{D} of 1 μM or less. In certain aspects, an anti-αvβ8 antibody binds to an epitope of αvβ8 that is conserved among αvβ8 from different species. In disclosed embodiments, an anti-αvβ8 antibody blocks the binding of αvβ8 and TGFβ1, including either latent TGFβ1 and mature TGFβ1. As used herein, “αvβ8” and αvβ6 may be written with or without Greek letters (e.g., avB8, αvB8, αvB6, or αvβ6). Likewise, TGFβ1 may be written “TGFβ1” and TGFβ3 may be written “TGFβ3”.

The term “antibody” herein is used in the broadest sense and encompasses various antibody structures, including, but not limited to, monoclonal antibodies, polyclonal antibodies, and multispecific antibodies (e.g., bispecific antibodies).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005). Fragments of the anti-αvβ8 antibodies disclosed herein may be selected for ability to bind αvβ8 and block the binding of αvβ8 to TGFβ 1 and/or TGFβ3.

As used herein, the term “epitope” denotes the site on an antigen to which an antibody binds. Epitopes can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an anti-αvβ8 antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

For the purposes herein, “atezolizumab” is an Fc-engineered, humanized, non-glycosylated IgG1 kappa immunoglobulin that binds PD-L1 and comprises the heavy chain sequence of SEQ ID NO:218 and the light chain sequence of SEQ ID NO:219. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances (INN)) Proposed INN: List 112, Vol. 28, No. 4, 2014, p. 488-489 and WHO Drug Information (International Nonproprietary Names for Pharmaceutical Substances (INN)), Recommended INN: List 74, Vol. 29, No. 3, 2015, p. 387.

Screening for antibodies binding to a particular epitope (i.e., those binding to the same epitope), such as an αvβ8 epitope, can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to αvβ8 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Competitive binding can also be used to easily determine whether an antibody binds to the same epitope of an antigen, or competes for binding with, a reference antibody. For example, an “antibody that binds to the same epitope” as a reference anti-αvβ8 antibody refers to an antibody that blocks binding of the reference anti-αvβ8 antibody to its antigen in a competition assay by 50% or more, and conversely, the reference antibody blocks binding of the antibody to its antigen in a competition assay by 50% or more. Also for example, to determine if an antibody binds to the same epitope as a reference anti-αvβ8 antibody, the reference antibody is allowed to bind to αvβ8 under saturating conditions. After removal of the excess of the reference anti-αvβ8 antibody, the ability of an anti-αvβ8 antibody in question to bind to αvβ8 is assessed. If the anti-αvβ8 antibody is able to bind to αvβ8 after saturation binding of the reference anti-αvβ8 antibody, it can be concluded that the anti-αvβ8 antibody in question binds to a different epitope than the reference anti-αvβ8 antibody. But, if the anti-αvβ8 antibody in question is not able to bind to αvβ8 after saturation binding of the reference anti-αvβ8 antibody, then the anti-αvβ8 antibody in question may bind to the same epitope as the epitope bound by the reference anti-αvβ8 antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to αvβ8, then it can be concluded that the anti-αvβ8 antibody in question and the reference anti-αvβ8 antibody compete for binding to αvβ8.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “cancer” refers to a disease caused by an uncontrolled division of abnormal cells in a part of the body. In some embodiments, the cancer is GBM, low-grade glioma, pheochromocytoma, adrenal cancer, mesothelioma, uveal melanoma, sarcoma, melanoma, cholangiocarcinoma, clear cell renal cell carcinoma (ccRCC), thymoma, papillary RCC, germ cell cancer, ovarian cancer, diffuse large B-cell lymphoma (DLBCL), or endometrial cancer. In specific embodiments, the cancer is ovarian cancer. In specific embodiments, the cancer is ccRCC. In embodiments, the cancer is a cancer exhibiting increased expression of αvβ8 and reduced expression of αvβ6 as compared with normal tissue. In embodiments, a cancer, such as ovarian cancer, is selected for treatment that has a ratio of avb8 expression to avb6 expression that is higher than comparable normal tissue. Because av integrin is a constituent of both avb8 and avb6, the expression of b8 and/or b6 integrin may be used as a surrogate of avb8 and/or avb6 expression. The cancer may be locally advanced or metastatic. In some instances, the cancer is locally advanced. In other instances, the cancer is metastatic. In some instances, the cancer may be unresectable (e.g., unresectable locally advanced or metastatic cancer).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG₄ antibody. For example, a disclosed anti-avb8 antibody can be an IgG₁, IgG₂, IgG₃, IgG₄, IgA₁, or IgA₂ isotype. In a particular an anti-avb8 antibody is of the IgG₁ isotype with the P329G, L234A and L235A mutation to reduce Fc-region effector function. In a particular an anti-avb8 antibody is of the IgG₂ isotype. In a particular an anti-avb8 antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991. In some embodiments, an antibody described herein comprises an Fc region of SEQ ID NO:201. In some embodiments, an antibody described herein comprises an Fc region of SEQ ID NO:220. In some embodiments, an antibody described herein comprises an Fc region of SEQ ID NO:203. In some embodiments, an antibody described herein comprises an Fc region of SEQ ID NO:221. In some embodiments, an antibody described herein comprises an Fc region according to SEQ ID NO:204.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-αvβ8 antibody” refers to one or more nucleic acid molecules encoding anti-αvβ8 antibody heavy and light chains (or fragments thereof), including such nucleic acid molecule(s) in a single vector or separate vectors, and such nucleic acid molecule(s) present at one or more locations in a host cell.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B37-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B37-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B37-H,” and “PDL1.”

The terms “Alpha v beta 8 integrin”, “Alpha v beta 8”, “avB8”, “aVB8”, “αvβ8”, “aVβ8”, “αVβ8”, or “αvβ8”, as used herein, refer synonymously to any native αvβ8 from any vertebrate source, including mammals such as primates (e.g., humans) and rodents (e.g., mice and rats), unless otherwise indicated. αvβ8 integrin is a transmembrane glycoprotein heterodimer composed of the αv and β8 subunits. αvβ8 has been detected in tumor cells of various carcinomas, including lung, ovarian, endometrial, melanoma, breast, prostate, colon, skin, and stomach. αvβ8 expression in human cancer cells is involved in the progression of epithelial malignancies; increased αvβ8 expression is also associated with decreased survival in non-small cell lung carcinoma, triple-negative basal-type breast cancer, and advanced ovarian cancer. Integrin αvβ8 can bind to numerous ECM proteins and is reported to be a primary receptor for latent TGF-01. αvβ8 integrin binding to the RGD motif contained in the LAP of latent TGF-β complexes mediates TGF-β activation and receptor pathway signaling. The term encompasses “full-length”, unprocessed αvβ8 as well as any form of αvβ8 that results from processing in the cell. The term also encompasses naturally occurring variants of αvβ8, e.g., splice variants or allelic variants. Exemplary protein sequences for αvβ8 can be found at Uniprot accession number P06756.2 for human and Uniprot accession number Q0VBD0 for murine. The αv protein in humans is encoded by the ITGAV gene. The amino acid sequence of an exemplary human αv integrin is shown in SEQ ID NO: 230 (MAFPPRRRLRLGPRGLPLLLSGLLLPLCRAFNLDVDSPAEYSGPEGSYFGFAVDFFVPSAS SRMFLLVGAPKANTTQPGIVEGGQVLKCDWSSTRRCQPIEFDATGNRDYAKDDPLEFKSH QWFGASVRSKQDKILACAPLYHWRTEMKQEREPVGTCFLQDGTKTVEYAPCRSQDIDAD GQGFCQGGFSIDFTKADRVLLGGPGSFYWQGQLISDQVAEIVSKYDPNVYSIKYNNQLAT RTAQAIFDDSYLGYSVAVGDFNGDGIDDFVSGVPRAARTLGMVYIYDGKNMSSLYNFTG EQMAAYFGFSVAATDINGDDYADVFIGAPLFMDRGSDGKLQEVGQVSVSLQRASGDFQT TKLNGFEVFARFGSAIAPLGDLDQDGFNDIAIAAPYGGEDKKGIVYIFNGRSTGLNAVPSQ ILEGQWAARSMPPSFGYSMKGATDIDKNGYPDLIVGAFGVDRAILYRARPVITVNAGLEV YPSILNQDNKTCSLPGTALKVSCFNVRFCLKADGKGVLPRKLNFQVELLLDKLKQKGAIR RALFLYSRSPSHSKNMTISRGGLMQCEELIAYLRDESEFRDKLTPITIFMEYRLDYRTAADT TGLQPILNQFTPANISRQAHILLDCGEDNVCKPKLEVSVDSDQKKIYIGDDNPLTLIVKAQ NQGEGAYEAELIVSIPLQADFIGVVRNNEALARLSCAFKTENQTRQVVCDLGNPMKAGT QLLAGLRFSVHQQSEMDTSVKFDLQIQSSNLFDKVSPVVSHKVDLAVLAAVEIRGVSSPD HVFLPIPNWEHKENPETEEDVGPVVQHIYELRNNGPSSFSKAMLHLQWPYKYNNNTLLYI LHYDIDGPMNCTSDMEINPLRIKISSLQTTEKNDTVAGQGERDHLITKRDLALSEGDIHTL GCGVAQCLKIVCQVGRLDRGKSAILYVKSLLWTETFMNKENQNHSYSLKSSASFNVIEFP YKNLPIEDITNSTLVTTNVTWGIQPAPMPVPVWVIILAVLAGLLLLAVLVFVMYRMGFFK RVRPPQEEQEREQLQPHENGEGNSET), which can additionally be found at UniProt ID NO P06756 as available in the NCBI database at Gene ID:3685. The β8 protein in humans is encoded by the ITGB8 gene. The amino acid sequence of an exemplary human β8 integrin is shown in SEQ ID NO 231 (MCGSALAFFTAAFVCLQNDRRGPASFLWAAWVFSLVLGLGQGEDNRCASSNAASCARC LALGPECGWCVQEDFISGGSRSERCDIVSNLISKGCSVDSIEYPSVHVIIPTENEINTQVTPG EVSIQLRPGAEANFMLKVHPLKKYPVDLYYLVDVSASMHNNIEKLNSVGNDLSRKMAFF SRDFRLGFGSYVDKTVSPYISIHPERIHNQCSDYNLDCMPPHGYIHVLSLTENITEFEKAVH RQKISGNIDTPEGGFDAMLQAAVCESHIGWRKEAKRLLLVMTDQTSHLALDSKLAGIVVP NDGNCHLKNNVYVKSTTMEHPSLGQLSEKLIDNNINVIFAVQGKQFHWYKDLLPLLPGTI AGEIESKAANLNNLVVEAYQKLISEVKVQVENQVQGIYFNITAICPDGSRKPGMEGCRNV TSNDEVLFNVTVTMKKCDVTGGKNYAIIKPIGFNETAKIHIHRNCSCQCEDNRGPKGKCV DETFLDSKCFQCDENKCHFDEDQFSSESCKSHKDQPVCSGRGVCVCGKCSCHKIKLGKV YGKYCEKDDFSCPYHHGNLCAGHGECEAGRCQCFSGWEGDRCQCPSAAAQHCVNSKG QVCSGRGTCVCGRCECTDPRSIGRFCEHCPTCYTACKENWNCMQCLHPHNLSQAILDQC KTSCALMEQQHYVDQTSECFSSPSYLRIFFIIFIVTFLIGLLKVLIIRQVILQWNSNKIKSSSD YRVSASKKDKLILQSVCTRAVTYRREKPEEIKMDISKLNAHETFRCNF), which can additionally be found at UniProt ID NO P26012 as available on in the NCBI database at Gene ID: 3696.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “patient” refers to a human patient of any age. In some embodiments, the patient is an adult.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In one aspect, provided herein are antibodies, or antigen-binding portions thereof, which specifically bind αvβ8, such as human αvβ8, murine αvβ8, cynomolgus αvβ8, and/or rabbit αvβ8 (i.e., anti-αvβ8 antibodies, synonymously termed αvβ8 antibodies). In some embodiments, the antibodies or antigen-binding portions thereof do not significantly (e.g., do not specifically) bind αvβ6 (for example, in some embodiments, the antibodies or antigen-binding portions thereof bind αvβ8 with at least 10-fold greater affinity than αvβ6, such as at least 20-fold, at least 30-fold, at least 40-fold, at least 50-fold, at least 60-fold, at least 70-fold, at least 80-fold, at least 90-fold, at least 100-fold, at least 1000-fold, or more greater affinity for αvβ8 than αvβ6). The antibodies and antigen-binding portions thereof described herein are useful for the diagnosis or treatment of cancer, such as cancers expressing αvβ8 (including cancers which express higher levels of αvβ8 and lower levels of αvβ6, cancers expressing normal levels of αvβ8 and lower levels of αvβ6, and cancers expressing normal levels of αvβ8 and normal levels of αvβ6). In some embodiments, the cancer is ovarian cancer. In some embodiments, the cancer is triple-negative breast cancer (TNBC). In some embodiments, the cancer is non-small cell lung cancer (NSCLC). In some embodiments, the cancer is colorectal cancer. In some embodiments, the cancer is cholangiocarcinoma. In some embodiments, the cancer is endometrial cancer. In some embodiments, the cancer is kidney renal papillary cancer. In some embodiments, the cancer is bladder cancer. The antibodies described herein may be particularly useful for treating cancers that express normal or greater-than-normal levels of αvβ8 but express lower than normal levels of αvβ6. In some embodiments, the cancer is a cancer expressing a ratio of αvβ8 to αvβ6 that is greater than normal for the tissue, such as a ratio that is about 1.1 times, about 1.2 times, about 1.3 times, about 1.4 times, about 1.5 times, about 1.6 times, about 1.7 times, about 1.8 times, about 1.9 times, about 2.0 times, about 2.5 times, about 3.0 times, about 3.5 times, about 4.0 times, about 5.0 times, about 10 times, about 15 times, about 20 times, about 25 times, about 30 times, about 35 times, about 40 times, about 45 times, about 50 times, about 60 times, about 70 times, about 80 times, about 90 times, about 100 times, about 200 times, about 300 times, about 400 times, about 500 times, about 600 times, about 700 times, about 800 times, about 900 times, about 1000 times, or more greater. The antibodies and antigen-binding portions thereof are also useful for the treatment of said cancers in combination with a therapy comprising a PD-1 axis antagonist, such as a PD-1 binding antagonist or a PD-L1 binding antagonist, such as an anti-PD-1 or anti-PD-L1 antibody. In a preferred embodiment, the anti-PD-L1 antibody is atezolizumab.

### A. Exemplary Anti-αvβ8 Antibodies

In one aspect, the invention provides antibodies or antigen-binding portions thereof that bind to αvβ8. In one aspect, provided are isolated antibodies that bind to αvβ8. In one aspect, the invention provides antibodies that specifically bind to αvβ8. In certain aspects, an anti-αvβ8 antibody or antigen-binding portion thereof exhibits at least one of the following properties: (a) binds to human αvβ8 with a K_{D} of 1 nM or less, binds to murine αvβ8 with a K_{D} of 1 nM or less, and/or binds to cynomolgus αvβ8 with a K_{D} of 1 nM or less; (b) inhibits αvβ8-mediated activation of latent TGFβ1 (LTGFβ1) and TGFβ33 presented by or associated with human-leucine-rich-repeat-containing protein 32 (LRRC32), LRRC33, and/or latent TGFβ3-binding proteins (LTBPs); (c) blocks binding of TGF peptide to αvβ8; and/or (d) binds αvβ8 in the absence of divalent cations. In some embodiments, the K_{D} of the antibody is assessed by surface plasmon resonance. In some embodiments, the anti-αvβ8 antibodies or antigen-binding portions thereof described herein bind αvβ8 with at least 10-fold greater affinity than αvβ6, such as any of at least 10-fold, 20-fold, 30-fold, 40-fold, 50-fold, 100-fold, 200-fold, 300-fold, 400-fold, 500-fold, 750-fold, 1000-fold or more greater affinity than αvβ6.

TABLE 1
Exemplary CDR sequences according to Kabat

| Antibody | CDR-L1 | CDR-L2 | CDR-L3 | CDR-H1 | CDR-H2 | CDR-H3 |
|---|---|---|---|---|---|---|
| Rb.aVb8-65 | QASENINSLLA | RASTLAS | QSNYGFTGSGAFFYG | NYAMI | IISSSDRIWYASWVKG | SVSNSYFDGWNL |
| | (SEQ ID NO: 1) | (SEQ ID | (SEQ ID NO: 3) | (SEQ ID | (SEQ ID NO: 5) | (SEQ ID NO: 6) |
| | | NO: 2) | | NO: 4) | | |
| Hu.aVb8- | QASENINSLLA | RASTLAS | QSNYGFTGSGAFFYG | NYAMI | IISSSDRIWYASWVKG | SVSNSYFDGWNL |
| 65.H1L1 | (SEQ ID NO: 7) | (SEQ ID | (SEQ ID NO: 9) | (SEQ ID | (SEQ ID NO: 11) | (SEQ ID NO: 12) |
| | | NO: 8) | | NO: 10) | | |
| Hu.aVb8- | QASENINSLLA | RASTLAS | QSNYGFTGSGAFFYG | NYAMI | IISSSDRIWYASWVKG | SVSNSYFDGWNL |
| 65.H15L2 | (SEQ ID NO: 13) | (SEQ ID | (SEQ ID NO: 15) | (SEQ ID | (SEQ ID NO: 17) | (SEQ ID NO: 18) |
| | | NO: 14) | | NO: 16) | | |
| Hu.aVb8- | QASENIQSLLA | RASTLAS | QSNYGFTGSGAFFYG | NYAMI | IISSSDRIWYASWVKG | SVSNSYFDGWNL |
| 65.H15L2.QS | (SEQ ID NO: 19) | (SEQ ID | (SEQ ID NO: 21) | (SEQ ID | (SEQ ID NO: 23) | (SEQ ID NO: 24) |
| | | NO: 20) | | NO: 22) | | |
| Hu.aVb8- | QASENINALLA | RASTLAS | QSNYGFTGSGAFFYG | NYAMI | IISSSDRIWYASWVKG | SVSNSYFDGWNL |
| 65.H15L2.NA | (SEQ ID NO: 25) | (SEQ ID | (SEQ ID NO: 27) | (SEQ ID | (SEQ ID NO: 29) | (SEQ ID NO: 30) |
| | | NO: 26) | | NO: 28) | | |
| Hu.aVb8- | QASENINTLLA | RASTLAS | QSNYGFTGSGAFFYG | NYAMI | IISSSDRIWYASWVKG | SVSNSYFDGWNL |
| 65.H15L2.NT | (SEQ ID NO: 31) | (SEQ ID | (SEQ ID NO: 33) | (SEQ ID | (SEQ ID NO: 35) | (SEQ ID NO: 36) |
| | | NO: 32) | | NO: 34) | | |
| Rb.aVb8-92; | QSIKSVGDNKWLA | DASDLAS | AGSVSSGDSG | SYAMG | VIRKDDNTYYGNWAKG | ALNTYVGFPYFNI |
| Hu.aVb8- | (SEQ ID NO: 37) | (SEQ ID | (SEQ ID NO: 39) | (SEQ ID | (SEQ ID NO: 41) | (SEQ ID NO: 42) |
| 92.H1L1; | | NO: 38) | | NO: 40) | | |
| Hu.aVb8- | | | | | | |
| 92.H13L1 | | | | | | |

TABLE 2
Exemplary CDR sequences according to Chothia

| Antibody | CDR-L1 | CDR-L2 | CDR-L3 | CDR-H1 | CDR-H2 | CDR-H3 |
|---|---|---|---|---|---|---|
| Rb.aVb8-65 | QASENINSLLA | RASTLAS | QSNYGFTGSGAFFYG | GIDLSNY | SSSDR | SVSNSYFDGWNL |
| | (SEQ ID NO: 43) | (SEQ ID | (SEQ ID NO: 45) | (SEQ ID | (SEQ ID | (SEQ ID NO: 48) |
| | | NO: 44) | | NO: 46) | NO: 47) | |
| Hu.aVb8- | QASENINSLLA | RASTLAS | QSNYGFTGSGAFFYG | GIDLSNY | SSSDR | SVSNSYFDGWNL |
| 65.H1L1 | (SEQ ID NO: 49) | (SEQ ID | (SEQ ID NO: 51) | (SEQ ID | (SEQ ID | (SEQ ID NO: 54) |
| | | NO: 50) | | NO: 52) | NO: 53) | |
| Hu.aVb8- | QASENINSLLA | RASTLAS | QSNYGFTGSGAFFYG | GIDLSNY | SSSDR | SVSNSYFDGWNL |
| 65.H15L2 | (SEQ ID NO: 55) | (SEQ ID | (SEQ ID NO: 57) | (SEQ ID | (SEQ ID | (SEQ ID NO: 60) |
| | | NO: 56) | | NO: 58) | NO: 59) | |
| Hu.aVb8- | QASENINSLLA | RASTLAS | QSNYGFTGSGAFFYG | GIDLSNY | SSSDR | SVSNSYFDGWNL |
| 65.H15L2.QS | (SEQ ID NO: 61) | (SEQ ID | (SEQ ID NO: 63) | (SEQ ID | (SEQ ID | (SEQ ID NO: 66) |
| | | NO: 62) | | NO: 64) | NO: 65) | |
| Hu.aVb8- | QASENINSLLA | RASTLAS | QSNYGFTGSGAFFYG | GIDLSNY | SSSDR | SVSNSYFDGWNL |
| 65.H15L2.NA | (SEQ ID NO: 67) | (SEQ ID | (SEQ ID NO: 69) | (SEQ ID | (SEQ ID | (SEQ ID NO: 72) |
| | | NO: 68) | | NO: 70) | NO: 71) | |
| Hu.aVb8- | QASENINSLLA | RASTLAS | QSNYGFTGSGAFFYG | GIDLSNY | SSSDR | SVSNSYFDGWNL |
| 65.H15L2.NT | (SEQ ID NO: 73) | (SEQ ID | (SEQ ID NO: 75) | (SEQ ID | (SEQ ID | (SEQ ID NO: 78) |
| | | NO: 74) | | NO: 76) | NO: 77) | |
| Rb.aVb8-92; | QSIKSVGDNKWLA | DASDLAS | AGSVSSGDSG | GFSLSSY | RKDDN | ALNTYVGFPYFNI |
| Hu.aVb8- | (SEQ ID NO: 79) | (SEQ ID | (SEQ ID NO: 81) | (SEQ ID | (SEQ ID | (SEQ ID NO: 84) |
| 92.H1L1; | | NO: 80) | | NO: 82) | NO: 83) | |
| Hu.aVb8- | | | | | | |
| 92.H13L1 | | | | | | |

TABLE 3
Exemplary CDR sequences according to CONTACT

| Antibody | CDR-L1 | CDR-L2 | CDR-L3 | CDR-H1 | CDR-H2 | CDR-H3 |
|---|---|---|---|---|---|---|
| Rb.aVb8-65 | NSLLAWY | LLIYRASTL | QSNYGFTGSGAFF | SNYAMI | WIGIISSSDRIW | SRSVSNSYFDG |
| | (SEQ ID | A | Y | (SEQ ID | (SEQ ID NO: 89) | W |
| | NO: 85) | (SEQ ID | (SEQ ID NO: 87) | NO: 88) | | (SEQ ID NO: 90) |
| | | NO: 86) | | | | |
| Hu.aVb8- | NSLLAWY | LLIYRASTL | QSNYGFTGSGAFF | SNYAMI | WIGIISSSDRIW | SRSVSNSYFDG |
| 65.H1L1 | (SEQ ID | A | Y | (SEQ ID | (SEQ ID NO: 95) | W |
| | NO: 91) | (SEQ ID | (SEQ ID NO: 93) | NO: 94) | | (SEQ ID NO: 96) |
| | | NO: 92) | | | | |
| Hu.aVb8- | NSLLAWY | LLIYRASTL | QSNYGFTGSGAFF | SNYAMI | WIGIISSSDRIW | SRSVSNSYFDG |
| 65.H15L2 | (SEQ ID | A | Y | (SEQ ID | (SEQ ID NO: 101) | W |
| | NO: 97) | (SEQ ID | (SEQ ID NO: 99) | NO: 100) | | (SEQ ID NO: 102) |
| | | NO: 98) | | | | |
| Hu.aVb8- | NSLLAWY | LLIYRASTL | QSNYGFTGSGAFF | SNYAMI | WIGIISSSDRIW | SRSVSNSYFDG |
| 65.H15L2.Q | (SEQ ID | A | Y | (SEQ ID | (SEQ ID NO: 107) | W |
| S | NO: 103) | (SEQ ID | (SEQ ID NO: 105) | NO: 106) | | (SEQ ID NO: 108) |
| | | NO: 104) | | | | |
| Hu.aVb8- | NSLLAWY | LLIYRASTL | QSNYGFTGSGAFF | SNYAMI | WIGIISSSDRIW | SRSVSNSYFDG |
| 65.H15L2.N | (SEQ ID | A | Y | (SEQ ID | (SEQ ID NO: 113) | W |
| A | NO: 109) | (SEQ ID | (SEQ ID NO: 111) | NO: 112) | | (SEQ ID NO: 114) |
| | | NO: 110) | | | | |
| Hu.aVb8- | NSLLAWY | LLIYRASTL | QSNYGFTGSGAFF | SNYAMI | WIGIISSSDRIW | SRSVSNSYFDG |
| 65.H15L2.N | (SEQ ID | A | Y | (SEQ ID | (SEQ ID NO: 119) | W |
| T | NO: 115) | (SEQ ID | (SEQ ID NO: 117) | NO: 118) | | (SEQ ID NO: 120) |
| | | NO: 116) | | | | |
| Rb.aVb8-92; | NKWLAW | LLIYDASDL | AGSVSSGDS | SSYAM | WIGVIRKDDNT | ARALNTYVGFP |
| Hu.aVb8- | Y | A | (SEQ ID NO: 123) | G | Y | Y |
| 92.H1L1; | (SEQ ID | (SEQ ID | | (SEQ ID | (SEQ ID NO: 125) | (SEQ ID NO: 126) |
| Hu.aVb8- | NO: 121) | NO: 122) | | NO: 124) | | |
| 92.H13L1 | | | | | | |

TABLE 4
Exemplary variable region sequences.

| | | |
|---|---|---|
| SEQ ID NO: 150 | Rb.aVb8-65 light chain | DIVMTQTPASVEAAVGGTVTIKCQASENIN |
| | variable | SLLAWYQQKPGQPPKLLIYRASTLASGVPS |
| | | RFRGSGSGTQFTLTISDLECDDAATYYCOS |
| | | NYGFTGSGAFFYGFGGGTEVVVE |
| SEQ ID NO: 151 | Rb.aVb8-65 heavy chain | QSVEESGGRLVTPGTPLTLTCTVSGIDLSNY |
| | variable | AMIWVRQAPGKGLEWIGIISSSDRIWYASW |
| | | VKGRFTISKTSSTTVDLQMTSLTTEDTATY |
| | | FCSRSVSNSYFDGWNLWGPGTLVTVSS |
| SEQ ID NO: 152 | Hu.aVb8-65.H1L1 | DIQMTQSPSSVSASVGDRVTITCQASENINS |
| | light chain | LLAWYQQKPGKPPKLLIYRASTLASGVPSR |
| | variable region | FSGSGSGTDFTLTISSLQPEDFATYYCQSNY |
| | | GFTGSGAFFYGFGQGTKVEIK |
| SEQ ID NO: 153 | Hu.aVb8-65.H1L1 heavy | EQQLLESGGGLVQPGGSLRLSCAVSGIDLS |
| | chain variable region | NYAMIWVRQAPGKGLEWIGIISSSDRIWYA |
| | | SWVKGRFTISKDSSKTTVYLQMNSLRAED |
| | | TAVYFCSRSVSNSYFDGWNLWGPGTLVTV |
| | | SS |
| SEQ ID NO: 154 | Hu.aVb8-65.H15L2 light | DIQMTQSPSSVSASVGDRVTITCQASENINS |
| | chain variable region | LLAWYQQKPGKAPKLLIYRASTLASGVPS |
| | | RFSGSGSGTDFTLTISSLQPEDFATYYCQSN |
| | | YGFTGSGAFFYGFGQGTKVEIK |
| SEQ ID NO: 155 | Hu.aVb8-65.H15L2 heavy | EVQLLESGGGLVQPGGSLRLSCAASGIDLS |
| | chain variable region | NYAMIWVRQAPGKGLEWVGIISSSDRIWY |
| | | ASWVKGRFTISRDNSKNTLYLQMNSLRAE |
| | | DTAVYYCSRSVSNSYFDGWNLWGQGTLV |
| | | TVSS |
| SEQ ID NO: 156 | Hu.aVb8-65.H15L2.QS light | DIQMTQSPSSVSASVGDRVTITCQASENIQS |
| | chain variable region | LLAWYQQKPGKAPKLLIYRASTLASGVPS |
| | | RFSGSGSGTDFTLTISSLQPEDFATYYCQSN |
| | | YGFTGSGAFFYGFGQGTKVEIK |
| SEQ ID NO: 157 | Hu.aVb8-65.H15L2.QS heavy | EVQLLESGGGLVQPGGSLRLSCAASGIDLS |
| | chain variable region | NYAMIWVRQAPGKGLEWVGIISSSDRIWY |
| | | ASWVKGRFTISRDNSKNTLYLQMNSLRAE |
| | | DTAVYYCSRSVSNSYFDGWNLWGQGTLV |
| | | TVSS |
| SEQ ID NO: 158 | Hu.aVb8-65.H15L2.NA light | DIQMTQSPSSVSASVGDRVTITCQASENINA |
| | chain variable region | LLAWYQQKPGKAPKLLIYRASTLASGVPS |
| | | RFSGSGSGTDFTLTISSLQPEDFATYYCQSN |
| | | YGFTGSGAFFYGFGQGTKVEIK |
| SEQ ID NO: 159 | Hu.aVb8-65.H15L2.NA heavy | EVQLLESGGGLVQPGGSLRLSCAASGIDLS |
| | chain variable region | NYAMIWVRQAPGKGLEWVGIISSSDRIWY |
| | | ASWVKGRFTISRDNSKNTLYLQMNSLRAE |
| | | DTAVYYCSRSVSNSYFDGWNLWGQGTLV |
| | | TVSS |
| SEQ ID NO: 160 | Hu.aVb8-65.H15L2.NT light | DIQMTQSPSSVSASVGDRVTITCQASENINT |
| | chain variable region | LLAWYQQKPGKAPKLLIYRASTLASGVPS |
| | | RFSGSGSGTDFTLTISSLQPEDFATYYCQSN |
| | | YGFTGSGAFFYGFGQGTKVEIK |
| SEQ ID NO: 161 | Hu.aVb8-65.H15L2.NT heavy | EVQLLESGGGLVQPGGSLRLSCAASGIDLS |
| | chain variable region | NYAMIWVRQAPGKGLEWVGIISSSDRIWY |
| | | ASWVKGRFTISRDNSKNTLYLQMNSLRAE |
| | | DTAVYYCSRSVSNSYFDGWNLWGQGTLV |
| | | TVSS |
| SEQ ID NO: 162 | Rb.aVb8-92 light chain | AAVLTQTPSPVSAAVGGTVSISCQSIKSVG |
| | variable region | DNKWLAWYQQKPGQPPKLLIYDASDLAS |
| | | GVPSRFKGSGSRTQFTLTISDLQCDDAATY |
| | | YCAGSVSSGDSGFGGGTEVVVA |
| SEQ ID NO: 163 | Rb.aVb8-92 heavy chain | QSVEESGGRLVTPGTPLTLTCTVSGFSLSSY |
| | variable region | AMGWVRQAPGKGLEWIGVIRKDDNTYYG |
| | | NWAKGRFTISRTSTTVDLKMTSLTAADTA |
| | | TYFCARALNTYVGFPYFNIWGPGTLVTVSS |
| SEQ ID NO: 164 | Hu.aVb8-92.H1L1 | DAVLTQSPDSLAVSLGERATINCQSIKSVG |
| | light chain | DNKWLAWYQQKPGQPPKLLIYDASDLAS |
| | variable region | GVPDRFSGSGSRTDFTLTISSLQAEDVAVY |
| | | YCAGSVSSGDSGFGGGTKVEIK |
| SEQ ID NO: 165 | Hu.aVb8-92.H1L1 heavy | EQQLLESGGGLVQPGGSLRLSCAVSGFSLS |
| | chain variable region | SYAMGWVRQAPGKGLEWIGVIRKDDNTY |
| | | YGNWAKGRFTISRDSSKNTVYLQMNSLRA |
| | | EDTAVYFCARALNTYVGFPYFNIWGPGTL |
| | | VTVSS |
| SEQ ID NO: 166 | Hu.aVb8-92.H13L1 light | DAVLTQSPDSLAVSLGERATINCQSIKSVG |
| | chain variable region | DNKWLAWYQQKPGQPPKLLIYDASDLAS |
| | | GVPDRFSGSGSRTDFTLTISSLQAEDVAVY |
| | | YCAGSVSSGDSGFGGGTKVEIK |
| SEQ ID NO: 167 | Hu.aVb8-92.H13L1 heavy | EQQLLESGGGLVQPGGSLRLSCAASGFSLS |
| | chain variable region | SYAMGWVRQAPGKGLEWIGVIRKDDNTY |
| | | YGNWAKGRFTISRDSSKNTLYLQMNSLRA |
| | | EDTAVYFCARALNTYVGFPYFNIWGPGTL |
| | | VTVSS |

TABLE 5
Exemplary light chain and heavy chain sequences.

| | | Sequence (variable regions |
|---|---|---|
| | | underlined) |
| SEQ ID NO: 200 | Hu.aVb8-65.H15L2 light | DIQMTQSPSSVSASVGDRVTITCQASENINS |
| | chain | LLAWYQQKPGKAPKLLIYRASTLASGVPS |
| | | RFSGSGSGTDFTLTISSLQPEDFATYYCQSN |
| | | YGFTGSGAFFYGFGQGTKVEIKRTVAAPSV |
| | | FIFPPSDEQLKSGTASVVCLLNNFYPREAK |
| | | VQWKVDNALQSGNSQESVTEQDSKDSTYS |
| | | LSSTLTLSKADYEKHKVYACEVTHQGLSSP |
| | | VTKSFNRGEC |
| SEQ ID NO: 201 | Hu.aVb8-65.H15L2 heavy | EVQLLESGGGLVQPGGSLRLSCAASGIDLS |
| | chain | NYAMIWVRQAPGKGLEWVGIISSSDRIWY |
| | | ASWVKGRFTISRDNSKNTLYLQMNSLRAE |
| | | DTAVYYCSRSVSNSYFDGWNLWGQGTLVT |
| | | VSSASTKGPSVFPLAPSSKSTSGGTAALGC |
| | | LVKDYFPEPVTVSWNSGALTSGVHTFPAVL |
| | | QSSGLYSLSSVVTVPSSSLGTQTYICNVNH |
| | | KPSNTKVDKKVEPKSCDKTHTCPPCPAPEA |
| | | AGGPSVFLFPPKPKDTLMISRTPEVTCVVV |
| | | DVSHEDPEVKFNWYVDGVEVHNAKTKPR |
| | | EEQYNSTYRVVSVLTVLHQDWLNGKEYK |
| | | CKVSNKALGAPIEKTISKAKGQPREPQVYT |
| | | LPPSREEMTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: 220 | Hu.aVb8-65.H15L2 heavy | EVQLLESGGGLVQPGGSLRLSCAASGI |
| | chain without terminal | DLSNYAMIWVRQAPGKGLEWVGIISSS |
| | lysine | DRIWYASWVKGRFTISRDNSKNTLYLQ |
| | | MNSLRAEDTAVYYCSRSVSNSYFDGW |
| | | NLWGQGTLVTVSSASTKGPSVFPLAPS |
| | | SKSTSGGTAALGCLVKDYFPEPVTVSW |
| | | NSGALTSGVHTFPAVLQSSGLYSLSSV |
| | | VTVPSSSLGTQTYICNVNHKPSNTKVD |
| | | KKVEPKSCDKTHTCPPCPAPEAAGGPS |
| | | VFLFPPKPKDTLMISRTPEVTCVVVDVS |
| | | HEDPEVKFNWYVDGVEVHNAKTKPRE |
| | | EQYNSTYRVVSVLTVLHQDWLNGKEY |
| | | KCKVSNKALGAPIEKTISKAKGQPREP |
| | | QVYTLPPSREEMTKNQVSLTCLVKGFY |
| | | PSDIAVEWESNGQPENNYKTTPPVLDS |
| | | DGSFFLYSKLTVDKSRWQQGNVFSCSV |
| | | MHEALHNHYTQKSLSLSPG |
| SEQ ID NO: 202 | Hu.aVb8-92.H13L1 light | DAVLTQSPDSLAVSLGERATINCQSIKSVG |
| | chain | DNKWLAWYQQKPGQPPKLLIYDASDLAS |
| | | GVPDRESGSGSRTDFTLTISSLQAEDVAVY |
| | | YCAGSVSSGDSGFGGGTKVEIKRTVAAPSV |
| | | FIFPPSDEQLKSGTASVVCLLNNFYPREAK |
| | | VQWKVDNALQSGNSQESVTEQDSKDSTYS |
| | | LSSTLTLSKADYEKHKVYACEVTHQGLSSP |
| | | VTKSFNRGEC |
| SEQ ID NO: 203 | Hu.aVb8-92.H13L1 heavy | EQQLLESGGGLVQPGGSLRLSCAASGESLS |
| | chain | SYAMGWVRQAPGKGLEWIGVIRKDDNTY |
| | | YGNWAKGRFTISRDSSKNTLYLQMNSLRA |
| | | EDTAVYFCARALNTYVGFPYFNIWGPGTL |
| | | VTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALGAPIEKTISKAKGQPREPQVY |
| | | TLPPSREEMTKNQVSLTCLVKGFYPSDIAV |
| | | EWESNGQPENNYKTTPPVLDSDGSFFLYSK |
| | | LTVDKSRWQQGNVFSCSVMHEALHNHYT |
| | | QKSLSLSPGK |
| SEQ ID NO: 221 | Hu.aVb8-92.H13L1 heavy | EQQLLESGGGLVQPGGSLRLSCAASGF |
| | chain without terminal | SLSSYAMGWVRQAPGKGLEWIGVIRK |
| | | DDNTYYGNWAKGRFTISRDSSKNTLYL |
| | lysine | QMNSLRAEDTAVYFCARALNTYVGFP |
| | | YFNIWGPGTLVTVSSASTKGPSVFPLAP |
| | | SSKSTSGGTAALGCLVKDYFPEPVTVS |
| | | WNSGALTSGVHTFPAVLQSSGLYSLSS |
| | | VVTVPSSSLGTQTYICNVNHKPSNTKV |
| | | DKKVEPKSCDKTHTCPPCPAPEAAGGP |
| | | SVFLFPPKPKDTLMISRTPEVTCVVVDV |
| | | SHEDPEVKFNWYVDGVEVHNAKTKPR |
| | | EEQYNSTYRVVSVLTVLHQDWLNGKE |
| | | YKCKVSNKALGAPIEKTISKAKGQPRE |
| | | PQVYTLPPSREEMTKNQVSLTCLVKGF |
| | | YPSDIAVEWESNGQPENNYKTTPPVLD |
| | | SDGSFFLYSKLTVDKSRWQQGNVFSCS |
| | | VMHEALHNHYTQKSLSLSPG |
| SEQ ID NO: 204 | aVb8 Fc region | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPS |
| | | NTKVDKKVEPKSCDKTHTCPPCPAPEAAG |
| | | GPSVFLFPPKPKDTLMISRTPEVTCVVVDV |
| | | SHEDPEVKFNWYVDGVEVHNAKTKPREE |
| | | QYNSTYRVVSVLTVLHQDWLNGKEYKCK |
| | | VSNKALGAPIEKTISKAKGQPREPQVYTLP |
| | | PSREEMTKNQVSLTCLVKGFYPSDIAVEWE |
| | | SNGQPENNYKTTPPVLDSDGSFFLYSKLTV |
| | | DKSRWQQGNVFSCSVMHEALHNHYTQKS |
| | | LSLSPGK |

In one aspect, the invention provides an anti-αvβ8 antibody comprising at least one, at least two, at least three, at least four, at least five, or all six CDRs from the antibody designated Hu.aVb8-65.H1L1 according to the CDRs defined in Table 1, Table 2, or Table 3. In one aspect, the invention provides an anti-αvβ8 antibody comprising at least one, at least two, at least three, at least four, at least five, or all six CDRs from the antibody designated Hu.aVb8-65.H15L2 according to the CDRs defined in Table 1, Table 2, or Table 3. In one aspect, the invention provides an anti-αvβ8 antibody comprising at least one, at least two, at least three, at least four, at least five, or all six CDRs from the antibody designated Hu.aVb8-65.H15L2.QS according to the CDRs defined in Table 1, Table 2, or Table 3. In one aspect, the invention provides an anti-αvβ8 antibody comprising at least one, at least two, at least three, at least four, at least five, or all six CDRs from the antibody designated Hu.aVb8-65.H15L2.NA according to the CDRs defined in Table 1, Table 2, or Table 3. In one aspect, the invention provides an anti-αvβ8 antibody comprising at least one, at least two, at least three, at least four, at least five, or all six CDRs from the antibody designated Hu.aVb8-65.H15L2.NT according to the CDRs defined in Table 1, Table 2, or Table 3. In one aspect, the invention provides an anti-αvβ8 antibody comprising at least one, at least two, at least three, at least four, at least five, or all six CDRs from the antibody designated Hu.aVb8-92.H1L1 according to the CDRs defined in Table 1, Table 2, or Table 3. In one aspect, the invention provides an anti-αvβ8 antibody comprising at least one, at least two, at least three, at least four, at least five, or all six CDRs from the antibody designated Hu.aVb8-92.H13L1 according to the CDRs defined in Table 1, Table 2, or Table 3.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:1; (b) CDR-L2 comprising the amino acid sequence of SEQ ID NO:2; (c) CDR-L3 comprising the amino acid sequence of SEQ ID NO:3; (d) CDR-H1 comprising the amino acid sequence of SEQ ID NO:4; (e) CDR-H2 comprising the amino acid sequence of SEQ ID NO:5; and (f) CDR-H3 comprising the amino acid sequence of SEQ ID NO:6.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:7; (b) CDR-L2 comprising the amino acid sequence of SEQ ID NO:8; (c) CDR-L3 comprising the amino acid sequence of SEQ ID NO:9; (d) CDR-H1 comprising the amino acid sequence of SEQ ID NO:10; (e) CDR-H2 comprising the amino acid sequence of SEQ ID NO:11; and (f) CDR-H3 comprising the amino acid sequence of SEQ ID NO:12.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:13; (b) CDR-L2 comprising the amino acid sequence of SEQ ID NO:14; (c) CDR-L3 comprising the amino acid sequence of SEQ ID NO:15; (d) CDR-H1 comprising the amino acid sequence of SEQ ID NO:16; (e) CDR-H2 comprising the amino acid sequence of SEQ ID NO:17; and (f) CDR-H3 comprising the amino acid sequence of SEQ ID NO:18.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:19; (b) CDR-L2 comprising the amino acid sequence of SEQ ID NO:20; (c) CDR-L3 comprising the amino acid sequence of SEQ ID NO:21; (d) CDR-H1 comprising the amino acid sequence of SEQ ID NO:22; (e) CDR-H2 comprising the amino acid sequence of SEQ ID NO:23; and (f) CDR-H3 comprising the amino acid sequence of SEQ ID NO:24.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:25; (b) CDR-L2 comprising the amino acid sequence of SEQ ID NO:26; (c) CDR-L3 comprising the amino acid sequence of SEQ ID NO:27; (d) CDR-H1 comprising the amino acid sequence of SEQ ID NO:28; (e) CDR-H2 comprising the amino acid sequence of SEQ ID NO:29; and (f) CDR-H3 comprising the amino acid sequence of SEQ ID NO:30.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:31; (b) CDR-L2 comprising the amino acid sequence of SEQ ID NO:32; (c) CDR-L3 comprising the amino acid sequence of SEQ ID NO:33; (d) CDR-H1 comprising the amino acid sequence of SEQ ID NO:34; (e) CDR-H2 comprising the amino acid sequence of SEQ ID NO:35; and (f) CDR-H3 comprising the amino acid sequence of SEQ ID NO:36.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:37; (b) CDR-L2 comprising the amino acid sequence of SEQ ID NO:38; (c) CDR-L3 comprising the amino acid sequence of SEQ ID NO:39; (d) CDR-H1 comprising the amino acid sequence of SEQ ID NO:40; (e) CDR-H2 comprising the amino acid sequence of SEQ ID NO:41; and (f) CDR-H3 comprising the amino acid sequence of SEQ ID NO:42.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:43; (b) CDR-L2 comprising the amino acid sequence of SEQ ID NO:44; (c) CDR-L3 comprising the amino acid sequence of SEQ ID NO:45; (d) CDR-H1 comprising the amino acid sequence of SEQ ID NO:46; (e) CDR-H2 comprising the amino acid sequence of SEQ ID NO:47; and (f) CDR-H3 comprising the amino acid sequence of SEQ ID NO:48.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:49; (b) CDR-L2 comprising the amino acid sequence of SEQ ID NO:50; (c) CDR-L3 comprising the amino acid sequence of SEQ ID NO:51; (d) CDR-H1 comprising the amino acid sequence of SEQ ID NO:52; (e) CDR-H2 comprising the amino acid sequence of SEQ ID NO:53; and (f) CDR-H3 comprising the amino acid sequence of SEQ ID NO:54.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:55; (b) CDR-L2 comprising the amino acid sequence of SEQ ID NO:56; (c) CDR-L3 comprising the amino acid sequence of SEQ ID NO:57; (d) CDR-H1 comprising the amino acid sequence of SEQ ID NO:58; (e) CDR-H2 comprising the amino acid sequence of SEQ ID NO:59; and (f) CDR-H3 comprising the amino acid sequence of SEQ ID NO:60.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:61; (b) CDR-L2 comprising the amino acid sequence of SEQ ID NO:62; (c) CDR-L3 comprising the amino acid sequence of SEQ ID NO:63; (d) CDR-H1 comprising the amino acid sequence of SEQ ID NO:64; (e) CDR-H2 comprising the amino acid sequence of SEQ ID NO:65; and (f) CDR-H3 comprising the amino acid sequence of SEQ ID NO:66.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:67; (b) CDR-L2 comprising the amino acid sequence of SEQ ID NO:68; (c) CDR-L3 comprising the amino acid sequence of SEQ ID NO:69; (d) CDR-H1 comprising the amino acid sequence of SEQ ID NO:70; (e) CDR-H2 comprising the amino acid sequence of SEQ ID NO:71; and (f) CDR-H3 comprising the amino acid sequence of SEQ ID NO:72.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:73; (b) CDR-L2 comprising the amino acid sequence of SEQ ID NO:74; (c) CDR-L3 comprising the amino acid sequence of SEQ ID NO:75; (d) CDR-H1 comprising the amino acid sequence of SEQ ID NO:76; (e) CDR-H2 comprising the amino acid sequence of SEQ ID NO:77; and (f) CDR-H3 comprising the amino acid sequence of SEQ ID NO:78.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:79; (b) CDR-L2 comprising the amino acid sequence of SEQ ID NO:80; (c) CDR-L3 comprising the amino acid sequence of SEQ ID NO:81; (d) CDR-H1 comprising the amino acid sequence of SEQ ID NO:82; (e) CDR-H2 comprising the amino acid sequence of SEQ ID NO:83; and (f) CDR-H3 comprising the amino acid sequence of SEQ ID NO:84.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:85; (b) CDR-L2 comprising the amino acid sequence of SEQ ID NO:86; (c) CDR-L3 comprising the amino acid sequence of SEQ ID NO:87; (d) CDR-H1 comprising the amino acid sequence of SEQ ID NO:88; (e) CDR-H2 comprising the amino acid sequence of SEQ ID NO:89; and (f) CDR-H3 comprising the amino acid sequence of SEQ ID NO:90.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:91; (b) CDR-L2 comprising the amino acid sequence of SEQ ID NO:92; (c) CDR-L3 comprising the amino acid sequence of SEQ ID NO:93; (d) CDR-H1 comprising the amino acid sequence of SEQ ID NO:94; (e) CDR-H2 comprising the amino acid sequence of SEQ ID NO:95; and (f) CDR-H3 comprising the amino acid sequence of SEQ ID NO:96.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:97; (b) CDR-L2 comprising the amino acid sequence of SEQ ID NO:98; (c) CDR-L3 comprising the amino acid sequence of SEQ ID NO:99; (d) CDR-H1 comprising the amino acid sequence of SEQ ID NO:100; (e) CDR-H2 comprising the amino acid sequence of SEQ ID NO:101; and (f) CDR-H3 comprising the amino acid sequence of SEQ ID NO:102.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:103; (b) CDR-L2 comprising the amino acid sequence of SEQ ID NO:104; (c) CDR-L3 comprising the amino acid sequence of SEQ ID NO:105; (d) CDR-H1 comprising the amino acid sequence of SEQ ID NO:106; (e) CDR-H2 comprising the amino acid sequence of SEQ ID NO:107; and (f) CDR-H3 comprising the amino acid sequence of SEQ ID NO:108.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:109; (b) CDR-L2 comprising the amino acid sequence of SEQ ID NO:110; (c) CDR-L3 comprising the amino acid sequence of SEQ ID NO:111; (d) CDR-H1 comprising the amino acid sequence of SEQ ID NO:112; (e) CDR-H2 comprising the amino acid sequence of SEQ ID NO:113; and (f) CDR-H3 comprising the amino acid sequence of SEQ ID NO:114.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:115; (b) CDR-L2 comprising the amino acid sequence of SEQ ID NO:116; (c) CDR-L3 comprising the amino acid sequence of SEQ ID NO:117; (d) CDR-H1 comprising the amino acid sequence of SEQ ID NO:118; (e) CDR-H2 comprising the amino acid sequence of SEQ ID NO:119; and (f) CDR-H3 comprising the amino acid sequence of SEQ ID NO:120.

In one aspect, the invention provides an anti-αvβ8 antibody comprising at least one, at least two, at least three, at least four, at least five, or all six CDRs selected from (a) CDR-L1 comprising the amino acid sequence of SEQ ID NO:121; (b) CDR-L2 comprising the amino acid sequence of SEQ ID NO:122; (c) CDR-L3 comprising the amino acid sequence of SEQ ID NO:123; (d) CDR-H1 comprising the amino acid sequence of SEQ ID NO:124; (e) CDR-H2 comprising the amino acid sequence of SEQ ID NO:125; and (f) CDR-H3 comprising the amino acid sequence of SEQ ID NO:126.

In some embodiments, the antibody or antigen-binding fragment thereof comprises a CDR-L1, CDR-L2, and CDR-L3 from the variable light chain sequence set forth in SEQ ID NO:150. In some embodiments, the antibody or antigen-binding fragment thereof comprises a CDR-L1, CDR-L2, and CDR-L3 from the variable light chain sequence set forth in SEQ ID NO:152. In some embodiments, the antibody or antigen-binding fragment thereof comprises a CDR-L1, CDR-L2, and CDR-L3 from the variable light chain sequence set forth in SEQ ID NO:154. In some embodiments, the antibody or antigen-binding fragment thereof comprises a CDR-L1, CDR-L2, and CDR-L3 from the variable light chain sequence set forth in SEQ ID NO:156. In some embodiments, the antibody or antigen-binding fragment thereof comprises a CDR-L1, CDR-L2, and CDR-L3 from the variable light chain sequence set forth in SEQ ID NO:158. In some embodiments, the antibody or antigen-binding fragment thereof comprises a CDR-L1, CDR-L2, and CDR-L3 from the variable light chain sequence set forth in SEQ ID NO:160. In some embodiments, the antibody or antigen-binding fragment thereof comprises a CDR-L1, CDR-L2, and CDR-L3 from the variable light chain sequence set forth in SEQ ID NO:162. In some embodiments, the antibody or antigen-binding fragment thereof comprises a CDR-L1, CDR-L2, and CDR-L3 from the variable light chain sequence set forth in SEQ ID NO:164. In some embodiments, the antibody or antigen-binding fragment thereof comprises a CDR-L1, CDR-L2, and CDR-L3 from the variable light chain sequence set forth in SEQ ID NO:166.

In some embodiments, the antibody or antigen-binding fragment thereof comprises a CDR-H1, CDR-H2, and CDR-H3 from the heavy chain variable region set forth in SEQ ID NO:151. In some embodiments, the antibody or antigen-binding fragment thereof comprises a CDR-H1, CDR-H2, and CDR-H3 from the heavy chain variable region set forth in SEQ ID NO:153. In some embodiments, the antibody or antigen-binding fragment thereof comprises a CDR-H1, CDR-H2, and CDR-H3 from the heavy chain variable region set forth in SEQ ID NO:155. In some embodiments, the antibody or antigen-binding fragment thereof comprises a CDR-H1, CDR-H2, and CDR-H3 from the heavy chain variable region set forth in SEQ ID NO:157. In some embodiments, the antibody or antigen-binding fragment thereof comprises a CDR-H1, CDR-H2, and CDR-H3 from the heavy chain variable region set forth in SEQ ID NO:159. In some embodiments, the antibody or antigen-binding fragment thereof comprises a CDR-H1, CDR-H2, and CDR-H3 from the heavy chain variable region set forth in SEQ ID NO:161. In some embodiments, the antibody or antigen-binding fragment thereof comprises a CDR-H1, CDR-H2, and CDR-H3 from the heavy chain variable region set forth in SEQ ID NO:163. In some embodiments, the antibody or antigen-binding fragment thereof comprises a CDR-H1, CDR-H2, and CDR-H3 from the heavy chain variable region set forth in SEQ ID NO:165. In some embodiments, the antibody or antigen-binding fragment thereof comprises a CDR-H1, CDR-H2, and CDR-H3 from the heavy chain variable region set forth in SEQ ID NO:167.

In one aspect, the invention provides an anti-αvβ8 antibody comprising (a) CDR-L1 comprising the amino acid sequence of SEQ ID NO:1; (b) CDR-L2 comprising the amino acid sequence of SEQ ID NO:2; (c) CDR-L3 comprising the amino acid sequence of SEQ ID NO:3; (d) CDR-H1 comprising the amino acid sequence of SEQ ID NO:4; (e) CDR-H2 comprising the amino acid sequence of SEQ ID NO:5; and (f) CDR-H3 comprising the amino acid sequence of SEQ ID NO:6.

In one aspect, the invention provides an anti-αvβ8 antibody comprising (a) CDR-L1 comprising the amino acid sequence of SEQ ID NO:7; (b) CDR-L2 comprising the amino acid sequence of SEQ ID NO:8; (c) CDR-L3 comprising the amino acid sequence of SEQ ID NO:9; (d) CDR-H1 comprising the amino acid sequence of SEQ ID NO:10; (e) CDR-H2 comprising the amino acid sequence of SEQ ID NO:11; and (f) CDR-H3 comprising the amino acid sequence of SEQ ID NO:12.

In one aspect, the invention provides an anti-αvβ8 antibody comprising (a) CDR-L1 comprising the amino acid sequence of SEQ ID NO:13; (b) CDR-L2 comprising the amino acid sequence of SEQ ID NO:14; (c) CDR-L3 comprising the amino acid sequence of SEQ ID NO:15; (d) CDR-H1 comprising the amino acid sequence of SEQ ID NO:16; (e) CDR-H2 comprising the amino acid sequence of SEQ ID NO:17; and (f) CDR-H3 comprising the amino acid sequence of SEQ ID NO:18.

In one aspect, the invention provides an anti-αvβ8 antibody comprising (a) CDR-L1 comprising the amino acid sequence of SEQ ID NO:19; (b) CDR-L2 comprising the amino acid sequence of SEQ ID NO:20; (c) CDR-L3 comprising the amino acid sequence of SEQ ID NO:21; (d) CDR-H1 comprising the amino acid sequence of SEQ ID NO:22; (e) CDR-H2 comprising the amino acid sequence of SEQ ID NO:23; and (f) CDR-H3 comprising the amino acid sequence of SEQ ID NO:24.

In one aspect, the invention provides an anti-αvβ8 antibody comprising (a) CDR-L1 comprising the amino acid sequence of SEQ ID NO:25; (b) CDR-L2 comprising the amino acid sequence of SEQ ID NO:26; (c) CDR-L3 comprising the amino acid sequence of SEQ ID NO:27; (d) CDR-H1 comprising the amino acid sequence of SEQ ID NO:28; (e) CDR-H2 comprising the amino acid sequence of SEQ ID NO:29; and (f) CDR-H3 comprising the amino acid sequence of SEQ ID NO:30.

In one aspect, the invention provides an anti-αvβ8 antibody comprising (a) CDR-L1 comprising the amino acid sequence of SEQ ID NO:31; (b) CDR-L2 comprising the amino acid sequence of SEQ ID NO:32; (c) CDR-L3 comprising the amino acid sequence of SEQ ID NO:33; (d) CDR-H1 comprising the amino acid sequence of SEQ ID NO:34; (e) CDR-H2 comprising the amino acid sequence of SEQ ID NO:35; and (f) CDR-H3 comprising the amino acid sequence of SEQ ID NO:36.

In one aspect, the invention provides an anti-αvβ8 antibody comprising (a) CDR-L1 comprising the amino acid sequence of SEQ ID NO:37; (b) CDR-L2 comprising the amino acid sequence of SEQ ID NO:38; (c) CDR-L3 comprising the amino acid sequence of SEQ ID NO:39; (d) CDR-H1 comprising the amino acid sequence of SEQ ID NO:40; (e) CDR-H2 comprising the amino acid sequence of SEQ ID NO:41; and (f) CDR-H3 comprising the amino acid sequence of SEQ ID NO:42.

In one aspect, the invention provides an anti-αvβ8 antibody comprising (a) CDR-L1 comprising the amino acid sequence of SEQ ID NO:43; (b) CDR-L2 comprising the amino acid sequence of SEQ ID NO:44; (c) CDR-L3 comprising the amino acid sequence of SEQ ID NO:45; (d) CDR-H1 comprising the amino acid sequence of SEQ ID NO:46; (e) CDR-H2 comprising the amino acid sequence of SEQ ID NO:47; and (f) CDR-H3 comprising the amino acid sequence of SEQ ID NO:48.

In one aspect, the invention provides an anti-αvβ8 antibody comprising (a) CDR-L1 comprising the amino acid sequence of SEQ ID NO:49; (b) CDR-L2 comprising the amino acid sequence of SEQ ID NO:50; (c) CDR-L3 comprising the amino acid sequence of SEQ ID NO:51; (d) CDR-H1 comprising the amino acid sequence of SEQ ID NO:52; (e) CDR-H2 comprising the amino acid sequence of SEQ ID NO:53; and (f) CDR-H3 comprising the amino acid sequence of SEQ ID NO:54.

In one aspect, the invention provides an anti-αvβ8 antibody comprising (a) CDR-L1 comprising the amino acid sequence of SEQ ID NO:55; (b) CDR-L2 comprising the amino acid sequence of SEQ ID NO:56; (c) CDR-L3 comprising the amino acid sequence of SEQ ID NO:57; (d) CDR-H1 comprising the amino acid sequence of SEQ ID NO:58; (e) CDR-H2 comprising the amino acid sequence of SEQ ID NO:59; and (f) CDR-H3 comprising the amino acid sequence of SEQ ID NO:60.

In one aspect, the invention provides an anti-αvβ8 antibody comprising (a) CDR-L1 comprising the amino acid sequence of SEQ ID NO:61; (b) CDR-L2 comprising the amino acid sequence of SEQ ID NO:62; (c) CDR-L3 comprising the amino acid sequence of SEQ ID NO:63; (d) CDR-H1 comprising the amino acid sequence of SEQ ID NO:64; (e) CDR-H2 comprising the amino acid sequence of SEQ ID NO:65; and (f) CDR-H3 comprising the amino acid sequence of SEQ ID NO:66.

In one aspect, the invention provides an anti-αvβ8 antibody comprising (a) CDR-L1 comprising the amino acid sequence of SEQ ID NO:67; (b) CDR-L2 comprising the amino acid sequence of SEQ ID NO:68; (c) CDR-L3 comprising the amino acid sequence of SEQ ID NO:69; (d) CDR-H1 comprising the amino acid sequence of SEQ ID NO:70; (e) CDR-H2 comprising the amino acid sequence of SEQ ID NO:71; and (f) CDR-H3 comprising the amino acid sequence of SEQ ID NO:72.

In one aspect, the invention provides an anti-αvβ8 antibody comprising (a) CDR-L1 comprising the amino acid sequence of SEQ ID NO:73; (b) CDR-L2 comprising the amino acid sequence of SEQ ID NO:74; (c) CDR-L3 comprising the amino acid sequence of SEQ ID NO:75; (d) CDR-H1 comprising the amino acid sequence of SEQ ID NO:76; (e) CDR-H2 comprising the amino acid sequence of SEQ ID NO:77; and (f) CDR-H3 comprising the amino acid sequence of SEQ ID NO:78.

In one aspect, the invention provides an anti-αvβ8 antibody comprising (a) CDR-L1 comprising the amino acid sequence of SEQ ID NO:79; (b) CDR-L2 comprising the amino acid sequence of SEQ ID NO:80; (c) CDR-L3 comprising the amino acid sequence of SEQ ID NO:81; (d) CDR-H1 comprising the amino acid sequence of SEQ ID NO:82; (e) CDR-H2 comprising the amino acid sequence of SEQ ID NO:83; and (f) CDR-H3 comprising the amino acid sequence of SEQ ID NO:84.

In one aspect, the invention provides an anti-αvβ8 antibody comprising (a) CDR-L1 comprising the amino acid sequence of SEQ ID NO:85; (b) CDR-L2 comprising the amino acid sequence of SEQ ID NO:86; (c) CDR-L3 comprising the amino acid sequence of SEQ ID NO:87; (d) CDR-H1 comprising the amino acid sequence of SEQ ID NO:88; (e) CDR-H2 comprising the amino acid sequence of SEQ ID NO:89; and (f) CDR-H3 comprising the amino acid sequence of SEQ ID NO:90.

In one aspect, the invention provides an anti-αvβ8 antibody comprising (a) CDR-L1 comprising the amino acid sequence of SEQ ID NO:91; (b) CDR-L2 comprising the amino acid sequence of SEQ ID NO:92; (c) CDR-L3 comprising the amino acid sequence of SEQ ID NO:93; (d) CDR-H1 comprising the amino acid sequence of SEQ ID NO:94; (e) CDR-H2 comprising the amino acid sequence of SEQ ID NO:95; and (f) CDR-H3 comprising the amino acid sequence of SEQ ID NO:96.

In one aspect, the invention provides an anti-αvβ8 antibody comprising (a) CDR-L1 comprising the amino acid sequence of SEQ ID NO:97; (b) CDR-L2 comprising the amino acid sequence of SEQ ID NO:98; (c) CDR-L3 comprising the amino acid sequence of SEQ ID NO:99; (d) CDR-H1 comprising the amino acid sequence of SEQ ID NO:100; (e) CDR-H2 comprising the amino acid sequence of SEQ ID NO:101; and (f) CDR-H3 comprising the amino acid sequence of SEQ ID NO:102.

In one aspect, the invention provides an anti-αvβ8 antibody comprising (a) CDR-L1 comprising the amino acid sequence of SEQ ID NO:103; (b) CDR-L2 comprising the amino acid sequence of SEQ ID NO:104; (c) CDR-L3 comprising the amino acid sequence of SEQ ID NO:105; (d) CDR-H1 comprising the amino acid sequence of SEQ ID NO:106; (e) CDR-H2 comprising the amino acid sequence of SEQ ID NO:107; and (f) CDR-H3 comprising the amino acid sequence of SEQ ID NO:108.

In one aspect, the invention provides an anti-αvβ8 antibody comprising (a) CDR-L1 comprising the amino acid sequence of SEQ ID NO:109; (b) CDR-L2 comprising the amino acid sequence of SEQ ID NO:110; (c) CDR-L3 comprising the amino acid sequence of SEQ ID NO:111; (d) CDR-H1 comprising the amino acid sequence of SEQ ID NO:112; (e) CDR-H2 comprising the amino acid sequence of SEQ ID NO:113; and (f) CDR-H3 comprising the amino acid sequence of SEQ ID NO:114.

In one aspect, the invention provides an anti-αvβ8 antibody comprising (a) CDR-L1 comprising the amino acid sequence of SEQ ID NO:115; (b) CDR-L2 comprising the amino acid sequence of SEQ ID NO:116; (c) CDR-L3 comprising the amino acid sequence of SEQ ID NO:117; (d) CDR-H1 comprising the amino acid sequence of SEQ ID NO:118; (e) CDR-H2 comprising the amino acid sequence of SEQ ID NO:119; and (f) CDR-H3 comprising the amino acid sequence of SEQ ID NO:120.

In one aspect, the invention provides an anti-αvβ8 antibody comprising (a) CDR-L1 comprising the amino acid sequence of SEQ ID NO:121; (b) CDR-L2 comprising the amino acid sequence of SEQ ID NO:122; (c) CDR-L3 comprising the amino acid sequence of SEQ ID NO:123; (d) CDR-H1 comprising the amino acid sequence of SEQ ID NO:124; (e) CDR-H2 comprising the amino acid sequence of SEQ ID NO:125; and (f) CDR-H3 comprising the amino acid sequence of SEQ ID NO:126.

In some embodiments, the anti-αvβ8 antibody or antigen-binding fragment thereof comprises the CDR sequences of the VL of SEQ ID NO:150 and the CDR sequences of the VH of SEQ ID NO:151. In some embodiments, the anti-αvβ8 antibody or antigen-binding fragment thereof comprises the CDR sequences of the VL of SEQ ID NO:152 and the CDR sequences of the VH of SEQ ID NO:153. In some embodiments, the anti-αvβ8 antibody or antigen-binding fragment thereof comprises the CDR sequences of the VL of SEQ ID NO:154 and the CDR sequences of the VH of SEQ ID NO:155. In some embodiments, the anti-αvβ8 antibody or antigen-binding fragment thereof comprises the CDR sequences of the VL of SEQ ID NO:156 and the CDR sequences of the VH of SEQ ID NO:157. In some embodiments, the anti-αvβ8 antibody or antigen-binding fragment thereof comprises the CDR sequences of the VL of SEQ ID NO:158 and the CDR sequences of the VH of SEQ ID NO:159. In some embodiments, the anti-αvβ8 antibody or antigen-binding fragment thereof comprises the CDR sequences of the VL of SEQ ID NO:160 and the CDR sequences of the VH of SEQ ID NO:161. In some embodiments, the anti-αvβ8 antibody or antigen-binding fragment thereof comprises the CDR sequences of the VL of SEQ ID NO:162 and the CDR sequences of the VH of SEQ ID NO:163. In some embodiments, the anti-αvβ8 antibody or antigen-binding fragment thereof comprises the CDR sequences of the VL of SEQ ID NO:164 and the CDR sequences of the VH of SEQ ID NO:165. In some embodiments, the anti-αvβ8 antibody or antigen-binding fragment thereof comprises the CDR sequences of the VL of SEQ ID NO:166 and the CDR sequences of the VH of SEQ ID NO:167.

In one aspect, the invention provides an anti-αvβ8 antibody comprising (a) CDR-L1 comprising the amino acid sequence of SEQ ID NO:1; (b) CDR-L2 comprising the amino acid sequence of SEQ ID NO:2; (c) CDR-L3 comprising the amino acid sequence of SEQ ID NO:3; (d) CDR-H1 comprising the amino acid sequence of SEQ ID NO:4; (e) CDR-H2 comprising the amino acid sequence of SEQ ID NO:5; and (f) CDR-H3 comprising the amino acid sequence of SEQ ID NO:6, and a VH domain having at least 90%, 91%, 92%, 93%, 94%, 95%, 96%, 97%, 98%, 99%, or 100% sequence identity to the amino acid sequence of SEQ ID NO:151, and a VL domain having at least 90%, 91%, 92%, 93%, 94%, 95%, 96%, 97%, 98%, 99%, or 100% sequence identity to the amino acid sequence of SEQ ID NO:150. In one aspect, the VH domain has at least 95% sequence identity to the amino acid sequence of SEQ ID NO:151. In one aspect, the VL domain has at least 95% sequence identity to the amino acid sequence of SEQ ID NO:150.

In one aspect, the invention provides an anti-αvβ8 antibody comprising (a) CDR-L1 comprising the amino acid sequence of SEQ ID NO:7; (b) CDR-L2 comprising the amino acid sequence of SEQ ID NO:8; (c) CDR-L3 comprising the amino acid sequence of SEQ ID NO:9; (d) CDR-H1 comprising the amino acid sequence of SEQ ID NO:10; (e) CDR-H2 comprising the amino acid sequence of SEQ ID NO:11; and (f) CDR-H3 comprising the amino acid sequence of SEQ ID NO:12, and a VH domain having at least 90%, 91%, 92%, 93%, 94%, 95%, 96%, 97%, 98%, 99%, or 100% sequence identity to the amino acid sequence of SEQ ID NO:153, and a VL domain having at least 90%, 91%, 92%, 93%, 94%, 95%, 96%, 97%, 98%, 99%, or 100% sequence identity to the amino acid sequence of SEQ ID NO:152. In one aspect, the VH domain has at least 95% sequence identity to the amino acid sequence of SEQ ID NO:153. In one aspect, the VL domain has at least 95% sequence identity to the amino acid sequence of SEQ ID NO:152.

In one aspect, the invention provides an anti-αvβ8 antibody comprising (a) CDR-L1 comprising the amino acid sequence of SEQ ID NO:13; (b) CDR-L2 comprising the amino acid sequence of SEQ ID NO:14; (c) CDR-L3 comprising the amino acid sequence of SEQ ID NO:15; (d) CDR-H1 comprising the amino acid sequence of SEQ ID NO:16; (e) CDR-H2 comprising the amino acid sequence of SEQ ID NO:17; and (f) CDR-H3 comprising the amino acid sequence of SEQ ID NO:18, and a VH domain having at least 90%, 91%, 92%, 93%, 94%, 95%, 96%, 97%, 98%, 99%, or 100% sequence identity to the amino acid sequence of SEQ ID NO:155, and a VL domain having at least 90%, 91%, 92%, 93%, 94%, 95%, 96%, 97%, 98%, 99%, or 100% sequence identity to the amino acid sequence of SEQ ID NO:154. In one aspect, the VH domain has at least 95% sequence identity to the amino acid sequence of SEQ ID NO:155. In one aspect, the VL domain has at least 95% sequence identity to the amino acid sequence of SEQ ID NO:154.

In one aspect, the invention provides an anti-αvβ8 antibody comprising (a) CDR-L1 comprising the amino acid sequence of SEQ ID NO:19; (b) CDR-L2 comprising the amino acid sequence of SEQ ID NO:20; (c) CDR-L3 comprising the amino acid sequence of SEQ ID NO:21; (d) CDR-H1 comprising the amino acid sequence of SEQ ID NO:22; (e) CDR-H2 comprising the amino acid sequence of SEQ ID NO:23; and (f) CDR-H3 comprising the amino acid sequence of SEQ ID NO:24, and a VH domain having at least 90%, 91%, 92%, 93%, 94%, 95%, 96%, 97%, 98%, 99%, or 100% sequence identity to the amino acid sequence of SEQ ID NO:157, and a VL domain having at least 90%, 91%, 92%, 93%, 94%, 95%, 96%, 97%, 98%, 99%, or 100% sequence identity to the amino acid sequence of SEQ ID NO:156. In one aspect, the VH domain has at least 95% sequence identity to the amino acid sequence of SEQ ID NO:157. In one aspect, the VL domain has at least 95% sequence identity to the amino acid sequence of SEQ ID NO:156.

In one aspect, the invention provides an anti-αvβ8 antibody comprising (a) CDR-L1 comprising the amino acid sequence of SEQ ID NO:25; (b) CDR-L2 comprising the amino acid sequence of SEQ ID NO:26; (c) CDR-L3 comprising the amino acid sequence of SEQ ID NO:27; (d) CDR-H1 comprising the amino acid sequence of SEQ ID NO:28; (e) CDR-H2 comprising the amino acid sequence of SEQ ID NO:29; and (f) CDR-H3 comprising the amino acid sequence of SEQ ID NO:30, and a VH domain having at least 90%, 91%, 92%, 93%, 94%, 95%, 96%, 97%, 98%, 99%, or 100% sequence identity to the amino acid sequence of SEQ ID NO:159, and a VL domain having at least 90%, 91%, 92%, 93%, 94%, 95%, 96%, 97%, 98%, 99%, or 100% sequence identity to the amino acid sequence of SEQ ID NO:158. In one aspect, the VH domain has at least 95% sequence identity to the amino acid sequence of SEQ ID NO:159. In one aspect, the VL domain has at least 95% sequence identity to the amino acid sequence of SEQ ID NO:158.

In one aspect, the invention provides an anti-αvβ8 antibody comprising (a) CDR-L1 comprising the amino acid sequence of SEQ ID NO:31; (b) CDR-L2 comprising the amino acid sequence of SEQ ID NO:32; (c) CDR-L3 comprising the amino acid sequence of SEQ ID NO:33; (d) CDR-H1 comprising the amino acid sequence of SEQ ID NO:34; (e) CDR-H2 comprising the amino acid sequence of SEQ ID NO:35; and (f) CDR-H3 comprising the amino acid sequence of SEQ ID NO:36, and a VH domain having at least 90%, 91%, 92%, 93%, 94%, 95%, 96%, 97%, 98%, 99%, or 100% sequence identity to the amino acid sequence of SEQ ID NO:161, and a VL domain having at least 90%, 91%, 92%, 93%, 94%, 95%, 96%, 97%, 98%, 99%, or 100% sequence identity to the amino acid sequence of SEQ ID NO:160. In one aspect, the VH domain has at least 95% sequence identity to the amino acid sequence of SEQ ID NO:161. In one aspect, the VL domain has at least 95% sequence identity to the amino acid sequence of SEQ ID NO:160.

In one aspect, the invention provides an anti-αvβ8 antibody comprising (a) CDR-L1 comprising the amino acid sequence of SEQ ID NO:37; (b) CDR-L2 comprising the amino acid sequence of SEQ ID NO:38; (c) CDR-L3 comprising the amino acid sequence of SEQ ID NO:39; (d) CDR-H1 comprising the amino acid sequence of SEQ ID NO:40; (e) CDR-H2 comprising the amino acid sequence of SEQ ID NO:41; and (f) CDR-H3 comprising the amino acid sequence of SEQ ID NO:42, and a VH domain having at least 90%, 91%, 92%, 93%, 94%, 95%, 96%, 97%, 98%, 99%, or 100% sequence identity to the amino acid sequence of SEQ ID NO:163, and a VL domain having at least 90%, 91%, 92%, 93%, 94%, 95%, 96%, 97%, 98%, 99%, or 100% sequence identity to the amino acid sequence of SEQ ID NO:162. In one aspect, the VH domain has at least 95% sequence identity to the amino acid sequence of SEQ ID NO:163. In one aspect, the VL domain has at least 95% sequence identity to the amino acid sequence of SEQ ID NO:162.

In one aspect, the invention provides an anti-αvβ8 antibody comprising (a) CDR-L1 comprising the amino acid sequence of SEQ ID NO:37; (b) CDR-L2 comprising the amino acid sequence of SEQ ID NO:38; (c) CDR-L3 comprising the amino acid sequence of SEQ ID NO:39; (d) CDR-H1 comprising the amino acid sequence of SEQ ID NO:40; (e) CDR-H2 comprising the amino acid sequence of SEQ ID NO:41; and (f) CDR-H3 comprising the amino acid sequence of SEQ ID NO:42, and a VH domain having at least 90%, 91%, 92%, 93%, 94%, 95%, 96%, 97%, 98%, 99%, or 100% sequence identity to the amino acid sequence of SEQ ID NO:165, and a VL domain having at least 90%, 91%, 92%, 93%, 94%, 95%, 96%, 97%, 98%, 99%, or 100% sequence identity to the amino acid sequence of SEQ ID NO:164. In one aspect, the VH domain has at least 95% sequence identity to the amino acid sequence of SEQ ID NO:165. In one aspect, the VL domain has at least 95% sequence identity to the amino acid sequence of SEQ ID NO:164.

In one aspect, the invention provides an anti-αvβ8 antibody comprising (a) CDR-L1 comprising the amino acid sequence of SEQ ID NO:37; (b) CDR-L2 comprising the amino acid sequence of SEQ ID NO:38; (c) CDR-L3 comprising the amino acid sequence of SEQ ID NO:39; (d) CDR-H1 comprising the amino acid sequence of SEQ ID NO:40; (e) CDR-H2 comprising the amino acid sequence of SEQ ID NO:41; and (f) CDR-H3 comprising the amino acid sequence of SEQ ID NO:42, and a VH domain having at least 90%, 91%, 92%, 93%, 94%, 95%, 96%, 97%, 98%, 99%, or 100% sequence identity to the amino acid sequence of SEQ ID NO:167, and a VL domain having at least 90%, 91%, 92%, 93%, 94%, 95%, 96%, 97%, 98%, 99%, or 100% sequence identity to the amino acid sequence of SEQ ID NO:166. In one aspect, the VH domain has at least 95% sequence identity to the amino acid sequence of SEQ ID NO:167. In one aspect, the VL domain has at least 95% sequence identity to the amino acid sequence of SEQ ID NO:166.

In one aspect, the invention provides an anti-αvβ8 antibody comprising (a) CDR-L1 comprising the amino acid sequence of SEQ ID NO:43; (b) CDR-L2 comprising the amino acid sequence of SEQ ID NO:44; (c) CDR-L3 comprising the amino acid sequence of SEQ ID NO:45; (d) CDR-H1 comprising the amino acid sequence of SEQ ID NO:46; (e) CDR-H2 comprising the amino acid sequence of SEQ ID NO:47; and (f) CDR-H3 comprising the amino acid sequence of SEQ ID NO:48, and a VH domain having at least 90%, 91%, 92%, 93%, 94%, 95%, 96%, 97%, 98%, 99%, or 100% sequence identity to the amino acid sequence of SEQ ID NO:151, and a VL domain having at least 90%, 91%, 92%, 93%, 94%, 95%, 96%, 97%, 98%, 99%, or 100% sequence identity to the amino acid sequence of SEQ ID NO:150. In one aspect, the VH domain has at least 95% sequence identity to the amino acid sequence of SEQ ID NO:151. In one aspect, the VL domain has at least 95% sequence identity to the amino acid sequence of SEQ ID NO:150.

In one aspect, the invention provides an anti-αvβ8 antibody comprising (a) CDR-L1 comprising the amino acid sequence of SEQ ID NO:49; (b) CDR-L2 comprising the amino acid sequence of SEQ ID NO:50; (c) CDR-L3 comprising the amino acid sequence of SEQ ID NO:51; (d) CDR-H1 comprising the amino acid sequence of SEQ ID NO:52; (e) CDR-H2 comprising the amino acid sequence of SEQ ID NO:53; and (f) CDR-H3 comprising the amino acid sequence of SEQ ID NO:54, and a VH domain having at least 90%, 91%, 92%, 93%, 94%, 95%, 96%, 97%, 98%, 99%, or 100% sequence identity to the amino acid sequence of SEQ ID NO:153, and a VL domain having at least 90%, 91%, 92%, 93%, 94%, 95%, 96%, 97%, 98%, 99%, or 100% sequence identity to the amino acid sequence of SEQ ID NO:152. In one aspect, the VH domain has at least 95% sequence identity to the amino acid sequence of SEQ ID NO:153. In one aspect, the VL domain has at least 95% sequence identity to the amino acid sequence of SEQ ID NO:152.

In one aspect, the invention provides an anti-αvβ8 antibody comprising (a) CDR-L1 comprising the amino acid sequence of SEQ ID NO:55; (b) CDR-L2 comprising the amino acid sequence of SEQ ID NO:56; (c) CDR-L3 comprising the amino acid sequence of SEQ ID NO:57; (d) CDR-H1 comprising the amino acid sequence of SEQ ID NO:58; (e) CDR-H2 comprising the amino acid sequence of SEQ ID NO:59; and (f) CDR-H3 comprising the amino acid sequence of SEQ ID NO:60, and a VH domain having at least 90%, 91%, 92%, 93%, 94%, 95%, 96%, 97%, 98%, 99%, or 100% sequence identity to the amino acid sequence of SEQ ID NO:155, and a VL domain having at least 90%, 91%, 92%, 93%, 94%, 95%, 96%, 97%, 98%, 99%, or 100% sequence identity to the amino acid sequence of SEQ ID NO:154. In one aspect, the VH domain has at least 95% sequence identity to the amino acid sequence of SEQ ID NO:155. In one aspect, the VL domain has at least 95% sequence identity to the amino acid sequence of SEQ ID NO:154.

In one aspect, the invention provides an anti-αvβ8 antibody comprising (a) CDR-L1 comprising the amino acid sequence of SEQ ID NO:61; (b) CDR-L2 comprising the amino acid sequence of SEQ ID NO:62; (c) CDR-L3 comprising the amino acid sequence of SEQ ID NO:63; (d) CDR-H1 comprising the amino acid sequence of SEQ ID NO:64; (e) CDR-H2 comprising the amino acid sequence of SEQ ID NO:65; and (f) CDR-H3 comprising the amino acid sequence of SEQ ID NO:66, and a VH domain having at least 90%, 91%, 92%, 93%, 94%, 95%, 96%, 97%, 98%, 99%, or 100% sequence identity to the amino acid sequence of SEQ ID NO:157, and a VL domain having at least 90%, 91%, 92%, 93%, 94%, 95%, 96%, 97%, 98%, 99%, or 100% sequence identity to the amino acid sequence of SEQ ID NO:156. In one aspect, the VH domain has at least 95% sequence identity to the amino acid sequence of SEQ ID NO:157. In one aspect, the VL domain has at least 95% sequence identity to the amino acid sequence of SEQ ID NO:156.

In one aspect, the invention provides an anti-αvβ8 antibody comprising (a) CDR-L1 comprising the amino acid sequence of SEQ ID NO:67; (b) CDR-L2 comprising the amino acid sequence of SEQ ID NO:68; (c) CDR-L3 comprising the amino acid sequence of SEQ ID NO:69; (d) CDR-H1 comprising the amino acid sequence of SEQ ID NO:70; (e) CDR-H2 comprising the amino acid sequence of SEQ ID NO:71; and (f) CDR-H3 comprising the amino acid sequence of SEQ ID NO:72, and a VH domain having at least 90%, 91%, 92%, 93%, 94%, 95%, 96%, 97%, 98%, 99%, or 100% sequence identity to the amino acid sequence of SEQ ID NO:159, and a VL domain having at least 90%, 91%, 92%, 93%, 94%, 95%, 96%, 97%, 98%, 99%, or 100% sequence identity to the amino acid sequence of SEQ ID NO:158. In one aspect, the VH domain has at least 95% sequence identity to the amino acid sequence of SEQ ID NO:159. In one aspect, the VL domain has at least 95% sequence identity to the amino acid sequence of SEQ ID NO:158.

In one aspect, the invention provides an anti-αvβ8 antibody comprising (a) CDR-L1 comprising the amino acid sequence of SEQ ID NO:73; (b) CDR-L2 comprising the amino acid sequence of SEQ ID NO:74; (c) CDR-L3 comprising the amino acid sequence of SEQ ID NO:75; (d) CDR-H1 comprising the amino acid sequence of SEQ ID NO:76; (e) CDR-H2 comprising the amino acid sequence of SEQ ID NO:77; and (f) CDR-H3 comprising the amino acid sequence of SEQ ID NO:78, and a VH domain having at least 90%, 91%, 92%, 93%, 94%, 95%, 96%, 97%, 98%, 99%, or 100% sequence identity to the amino acid sequence of SEQ ID NO:161, and a VL domain having at least 90%, 91%, 92%, 93%, 94%, 95%, 96%, 97%, 98%, 99%, or 100% sequence identity to the amino acid sequence of SEQ ID NO:160. In one aspect, the VH domain has at least 95% sequence identity to the amino acid sequence of SEQ ID NO:161. In one aspect, the VL domain has at least 95% sequence identity to the amino acid sequence of SEQ ID NO:160.

In one aspect, the invention provides an anti-αvβ8 antibody comprising (a) CDR-L1 comprising the amino acid sequence of SEQ ID NO:79; (b) CDR-L2 comprising the amino acid sequence of SEQ ID NO:80; (c) CDR-L3 comprising the amino acid sequence of SEQ ID NO:81; (d) CDR-H1 comprising the amino acid sequence of SEQ ID NO:82; (e) CDR-H2 comprising the amino acid sequence of SEQ ID NO:83; and (f) CDR-H3 comprising the amino acid sequence of SEQ ID NO:84, and a VH domain having at least 90%, 91%, 92%, 93%, 94%, 95%, 96%, 97%, 98%, 99%, or 100% sequence identity to the amino acid sequence of SEQ ID NO:163, and a VL domain having at least 90%, 91%, 92%, 93%, 94%, 95%, 96%, 97%, 98%, 99%, or 100% sequence identity to the amino acid sequence of SEQ ID NO:162. In one aspect, the VH domain has at least 95% sequence identity to the amino acid sequence of SEQ ID NO:163. In one aspect, the VL domain has at least 95% sequence identity to the amino acid sequence of SEQ ID NO:162.

In one aspect, the invention provides an anti-αvβ8 antibody comprising (a) CDR-L1 comprising the amino acid sequence of SEQ ID NO:79; (b) CDR-L2 comprising the amino acid sequence of SEQ ID NO:80; (c) CDR-L3 comprising the amino acid sequence of SEQ ID NO:81; (d) CDR-H1 comprising the amino acid sequence of SEQ ID NO:82; (e) CDR-H2 comprising the amino acid sequence of SEQ ID NO:83; and (f) CDR-H3 comprising the amino acid sequence of SEQ ID NO:84, and a VH domain having at least 90%, 91%, 92%, 93%, 94%, 95%, 96%, 97%, 98%, 99%, or 100% sequence identity to the amino acid sequence of SEQ ID NO:165, and a VL domain having at least 90%, 91%, 92%, 93%, 94%, 95%, 96%, 97%, 98%, 99%, or 100% sequence identity to the amino acid sequence of SEQ ID NO:164. In one aspect, the VH domain has at least 95% sequence identity to the amino acid sequence of SEQ ID NO:165. In one aspect, the VL domain has at least 95% sequence identity to the amino acid sequence of SEQ ID NO:164.

In one aspect, the invention provides an anti-αvβ8 antibody comprising (a) CDR-L1 comprising the amino acid sequence of SEQ ID NO:79; (b) CDR-L2 comprising the amino acid sequence of SEQ ID NO:80; (c) CDR-L3 comprising the amino acid sequence of SEQ ID NO:81; (d) CDR-H1 comprising the amino acid sequence of SEQ ID NO:82; (e) CDR-H2 comprising the amino acid sequence of SEQ ID NO:83; and (f) CDR-H3 comprising the amino acid sequence of SEQ ID NO:84, and a VH domain having at least 90%, 91%, 92%, 93%, 94%, 95%, 96%, 97%, 98%, 99%, or 100% sequence identity to the amino acid sequence of SEQ ID NO:167, and a VL domain having at least 90%, 91%, 92%, 93%, 94%, 95%, 96%, 97%, 98%, 99%, or 100% sequence identity to the amino acid sequence of SEQ ID NO:166. In one aspect, the VH domain has at least 95% sequence identity to the amino acid sequence of SEQ ID NO:167. In one aspect, the VL domain has at least 95% sequence identity to the amino acid sequence of SEQ ID NO:166.

In one aspect, the invention provides an anti-αvβ8 antibody comprising (a) CDR-L1 comprising the amino acid sequence of SEQ ID NO:85; (b) CDR-L2 comprising the amino acid sequence of SEQ ID NO:86; (c) CDR-L3 comprising the amino acid sequence of SEQ ID NO:87; (d) CDR-H1 comprising the amino acid sequence of SEQ ID NO:88; (e) CDR-H2 comprising the amino acid sequence of SEQ ID NO:89; and (f) CDR-H3 comprising the amino acid sequence of SEQ ID NO:90, and a VH domain having at least 90%, 91%, 92%, 93%, 94%, 95%, 96%, 97%, 98%, 99%, or 100% sequence identity to the amino acid sequence of SEQ ID NO:151, and a VL domain having at least 90%, 91%, 92%, 93%, 94%, 95%, 96%, 97%, 98%, 99%, or 100% sequence identity to the amino acid sequence of SEQ ID NO:150. In one aspect, the VH domain has at least 95% sequence identity to the amino acid sequence of SEQ ID NO:151. In one aspect, the VL domain has at least 95% sequence identity to the amino acid sequence of SEQ ID NO:150.

In one aspect, the invention provides an anti-αvβ8 antibody comprising (a) CDR-L1 comprising the amino acid sequence of SEQ ID NO:91; (b) CDR-L2 comprising the amino acid sequence of SEQ ID NO:92; (c) CDR-L3 comprising the amino acid sequence of SEQ ID NO:93; (d) CDR-H1 comprising the amino acid sequence of SEQ ID NO:94; (e) CDR-H2 comprising the amino acid sequence of SEQ ID NO:95; and (f) CDR-H3 comprising the amino acid sequence of SEQ ID NO:96, and a VH domain having at least 90%, 91%, 92%, 93%, 94%, 95%, 96%, 97%, 98%, 99%, or 100% sequence identity to the amino acid sequence of SEQ ID NO:153, and a VL domain having at least 90%, 91%, 92%, 93%, 94%, 95%, 96%, 97%, 98%, 99%, or 100% sequence identity to the amino acid sequence of SEQ ID NO:152. In one aspect, the VH domain has at least 95% sequence identity to the amino acid sequence of SEQ ID NO:153. In one aspect, the VL domain has at least 95% sequence identity to the amino acid sequence of SEQ ID NO:152.

In one aspect, the invention provides an anti-αvβ8 antibody comprising (a) CDR-L1 comprising the amino acid sequence of SEQ ID NO:97; (b) CDR-L2 comprising the amino acid sequence of SEQ ID NO:98; (c) CDR-L3 comprising the amino acid sequence of SEQ ID NO:99; (d) CDR-H1 comprising the amino acid sequence of SEQ ID NO:100; (e) CDR-H2 comprising the amino acid sequence of SEQ ID NO:101; and (f) CDR-H3 comprising the amino acid sequence of SEQ ID NO:102, and a VH domain having at least 90%, 91%, 92%, 93%, 94%, 95%, 96%, 97%, 98%, 99%, or 100% sequence identity to the amino acid sequence of SEQ ID NO:155, and a VL domain having at least 90%, 91%, 92%, 93%, 94%, 95%, 96%, 97%, 98%, 99%, or 100% sequence identity to the amino acid sequence of SEQ ID NO:154. In one aspect, the VH domain has at least 95% sequence identity to the amino acid sequence of SEQ ID NO:155. In one aspect, the VL domain has at least 95% sequence identity to the amino acid sequence of SEQ ID NO:154.

In one aspect, the invention provides an anti-αvβ8 antibody comprising (a) CDR-L1 comprising the amino acid sequence of SEQ ID NO:103; (b) CDR-L2 comprising the amino acid sequence of SEQ ID NO:104; (c) CDR-L3 comprising the amino acid sequence of SEQ ID NO:105; (d) CDR-H1 comprising the amino acid sequence of SEQ ID NO:106; (e) CDR-H2 comprising the amino acid sequence of SEQ ID NO:107; and (f) CDR-H3 comprising the amino acid sequence of SEQ ID NO:108, and a VH domain having at least 90%, 91%, 92%, 93%, 94%, 95%, 96%, 97%, 98%, 99%, or 100% sequence identity to the amino acid sequence of SEQ ID NO:157, and a VL domain having at least 90%, 91%, 92%, 93%, 94%, 95%, 96%, 97%, 98%, 99%, or 100% sequence identity to the amino acid sequence of SEQ ID NO:156. In one aspect, the VH domain has at least 95% sequence identity to the amino acid sequence of SEQ ID NO:157. In one aspect, the VL domain has at least 95% sequence identity to the amino acid sequence of SEQ ID NO:156.

In one aspect, the invention provides an anti-αvβ8 antibody comprising (a) CDR-L1 comprising the amino acid sequence of SEQ ID NO:109; (b) CDR-L2 comprising the amino acid sequence of SEQ ID NO:110; (c) CDR-L3 comprising the amino acid sequence of SEQ ID NO:111; (d) CDR-H1 comprising the amino acid sequence of SEQ ID NO:112; (e) CDR-H2 comprising the amino acid sequence of SEQ ID NO:113; and (f) CDR-H3 comprising the amino acid sequence of SEQ ID NO:114, and a VH domain having at least 90%, 91%, 92%, 93%, 94%, 95%, 96%, 97%, 98%, 99%, or 100% sequence identity to the amino acid sequence of SEQ ID NO:159, and a VL domain having at least 90%, 91%, 92%, 93%, 94%, 95%, 96%, 97%, 98%, 99%, or 100% sequence identity to the amino acid sequence of SEQ ID NO:158. In one aspect, the VH domain has at least 95% sequence identity to the amino acid sequence of SEQ ID NO:159. In one aspect, the VL domain has at least 95% sequence identity to the amino acid sequence of SEQ ID NO:158.

In one aspect, the invention provides an anti-αvβ8 antibody comprising (a) CDR-L1 comprising the amino acid sequence of SEQ ID NO:115; (b) CDR-L2 comprising the amino acid sequence of SEQ ID NO:116; (c) CDR-L3 comprising the amino acid sequence of SEQ ID NO:117; (d) CDR-H1 comprising the amino acid sequence of SEQ ID NO:118; (e) CDR-H2 comprising the amino acid sequence of SEQ ID NO:119; and (f) CDR-H3 comprising the amino acid sequence of SEQ ID NO:120, and a VH domain having at least 90%, 91%, 92%, 93%, 94%, 95%, 96%, 97%, 98%, 99%, or 100% sequence identity to the amino acid sequence of SEQ ID NO:161, and a VL domain having at least 90%, 91%, 92%, 93%, 94%, 95%, 96%, 97%, 98%, 99%, or 100% sequence identity to the amino acid sequence of SEQ ID NO:160. In one aspect, the VH domain has at least 95% sequence identity to the amino acid sequence of SEQ ID NO:161. In one aspect, the VL domain has at least 95% sequence identity to the amino acid sequence of SEQ ID NO:160.

In one aspect, the invention provides an anti-αvβ8 antibody comprising (a) CDR-L1 comprising the amino acid sequence of SEQ ID NO:121; (b) CDR-L2 comprising the amino acid sequence of SEQ ID NO:122; (c) CDR-L3 comprising the amino acid sequence of SEQ ID NO:123; (d) CDR-H1 comprising the amino acid sequence of SEQ ID NO:124; (e) CDR-H2 comprising the amino acid sequence of SEQ ID NO:125; and (f) CDR-H3 comprising the amino acid sequence of SEQ ID NO:126, and a VH domain having at least 90%, 91%, 92%, 93%, 94%, 95%, 96%, 97%, 98%, 99%, or 100% sequence identity to the amino acid sequence of SEQ ID NO:163, and a VL domain having at least 90%, 91%, 92%, 93%, 94%, 95%, 96%, 97%, 98%, 99%, or 100% sequence identity to the amino acid sequence of SEQ ID NO:162. In one aspect, the VH domain has at least 95% sequence identity to the amino acid sequence of SEQ ID NO:163. In one aspect, the VL domain has at least 95% sequence identity to the amino acid sequence of SEQ ID NO:162.

In one aspect, the invention provides an anti-αvβ8 antibody comprising (a) CDR-L1 comprising the amino acid sequence of SEQ ID NO:121; (b) CDR-L2 comprising the amino acid sequence of SEQ ID NO:122; (c) CDR-L3 comprising the amino acid sequence of SEQ ID NO:123; (d) CDR-H1 comprising the amino acid sequence of SEQ ID NO:124; (e) CDR-H2 comprising the amino acid sequence of SEQ ID NO:125; and (f) CDR-H3 comprising the amino acid sequence of SEQ ID NO:126, and a VH domain having at least 90%, 91%, 92%, 93%, 94%, 95%, 96%, 97%, 98%, 99%, or 100% sequence identity to the amino acid sequence of SEQ ID NO:165, and a VL domain having at least 90%, 91%, 92%, 93%, 94%, 95%, 96%, 97%, 98%, 99%, or 100% sequence identity to the amino acid sequence of SEQ ID NO:164. In one aspect, the VH domain has at least 95% sequence identity to the amino acid sequence of SEQ ID NO:165. In one aspect, the VL domain has at least 95% sequence identity to the amino acid sequence of SEQ ID NO:164.

In one aspect, the invention provides an anti-αvβ8 antibody comprising (a) CDR-L1 comprising the amino acid sequence of SEQ ID NO:121; (b) CDR-L2 comprising the amino acid sequence of SEQ ID NO:122; (c) CDR-L3 comprising the amino acid sequence of SEQ ID NO:123; (d) CDR-H1 comprising the amino acid sequence of SEQ ID NO:124; (e) CDR-H2 comprising the amino acid sequence of SEQ ID NO:125; and (f) CDR-H3 comprising the amino acid sequence of SEQ ID NO:126, and a VH domain having at least 90%, 91%, 92%, 93%, 94%, 95%, 96%, 97%, 98%, 99%, or 100% sequence identity to the amino acid sequence of SEQ ID NO:167, and a VL domain having at least 90%, 91%, 92%, 93%, 94%, 95%, 96%, 97%, 98%, 99%, or 100% sequence identity to the amino acid sequence of SEQ ID NO:166. In one aspect, the VH domain has at least 95% sequence identity to the amino acid sequence of SEQ ID NO:167. In one aspect, the VL domain has at least 95% sequence identity to the amino acid sequence of SEQ ID NO:166.

In some embodiments, the anti-αvβ8 antibody comprises a light chain variable region (VL) sequence having at least 90%, 91%, 92%, 93%, 94%, 95%, 96%, 97%, 98%, 99%, or 100% sequence identity to the amino acid sequence of SEQ ID NO:150. In some embodiments, the anti-αvβ8 antibody comprises a VL sequence having at least 95% sequence identity to the amino acid sequence of SEQ ID NO:150. In some embodiments, the VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-αvβ8 antibody comprising that sequence retains the ability to bind to αvβ8. In certain aspects, a total of 1 to 10 amino acids have been substituted, inserted and/or deleted in SEQ ID NO:150. In certain aspects, substitutions, insertions, or deletions occur in regions outside the CDRs (i.e., in the FRs).

Optionally, the anti-αvβ8 antibody comprises the VL sequence in SEQ ID NO:150, including post-translational modifications of that sequence. In some embodiments, the anti-αvβ8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:151. In one aspect, the anti-αvβ8 antibody comprises a heavy chain variable domain (VH) sequence having at least 95% sequence identity to the amino acid sequence of SEQ ID NO:151. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-αvβ8 antibody comprising that sequence retains the ability to bind to αvβ8. In certain aspects, a total of 1 to 10 amino acids have been substituted, inserted and/or deleted in SEQ ID NO:151. In certain aspects, the substitutions, insertions, or deletions occur in regions outside the CDRs (i.e., in the FRs). Optionally, the anti-αvβ8 antibody comprises the VH sequence in SEQ ID NO:151, including post-translational modifications of that sequence.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:151 and SEQ ID NO:150, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:153 and SEQ ID NO:152, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:155 and SEQ ID NO:154, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:157 and SEQ ID NO:156, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:159 and SEQ ID NO:158, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:161 and SEQ ID NO:160, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:163 and SEQ ID NO:162, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:165 and SEQ ID NO:164, respectively, including post-translational modifications of those sequences.

In another aspect, an anti-αvβ8 antibody is provided, wherein the antibody comprises a VH sequence as in any of the aspects provided above, and a VL sequence as in any of the aspects provided above. In one aspect, the antibody comprises the VH and VL sequences in SEQ ID NO:167 and SEQ ID NO:166, respectively, including post-translational modifications of those sequences.

In a further aspect of the invention, an anti-αvβ8 antibody according to any of the above aspects is a monoclonal antibody, including a chimeric, humanized or human antibody. In one aspect, an anti-αvβ8 antibody is an antibody fragment, e.g., a Fv, Fab, Fab′, scFv, diabody, or F(ab′)2 fragment.

In another aspect, the antibody is a full length antibody, e.g., an intact full-length IgG1 antibody or other antibody class or isotype as defined herein.

In some embodiments, the antibody as described herein is of IgG1 isotype/subclass. In some embodiments, the antibody as described herein is of IgG1 isotype/subclass and is modified to reduce Fc-region effector function. In some embodiments, the antibody as described herein is of IgG1 isotype/subclass and comprises the amino acid substitutions L234A and L235A, with numbering according to the EU index of Kabat. In some embodiments, the antibody as described herein is of IgG1 isotype/subclass and comprises the amino acid substitution P329G, with numbering according to the EU index of Kabat. In some embodiments, the antibody described herein comprises the Fc region according to SEQ ID NO:204. In some embodiments, the antibody described herein comprises an Fc region exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:204. In some embodiments, the antibody described herein comprises the C-terminal glycine (Gly446). In some embodiments, the antibody described herein comprises the C-terminal glycine (Gly446) and the C-terminal lysine (Lys447).

In some embodiments, the antibody described herein comprises the light chain sequence of SEQ ID NO:200 and the heavy chain sequence of SEQ ID NO:201. In some embodiments, the antibody described herein comprises the light chain sequence of SEQ ID NO:200 and the heavy chain sequence of SEQ ID NO:220. In some embodiments, the antibody described herein comprises a light chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:200 and a heavy chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:201. In some embodiments, the antibody described herein comprises a light chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:200 and a heavy chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:220.

In some embodiments, the antibody described herein comprises the light chain sequence of SEQ ID NO:202 and the heavy chain sequence of SEQ ID NO:203. In some embodiments, the antibody described herein comprises the light chain sequence of SEQ ID NO:202 and the heavy chain sequence of SEQ ID NO:221. In some embodiments, the antibody described herein comprises a light chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:202 and a heavy chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:203. In some embodiments, the antibody described herein comprises a light chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:202 and a heavy chain sequence exhibiting at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:221.

In a further aspect, an anti-αvβ8 antibody according to any of the above aspects may incorporate any of the features, singly or in combination, as described in the following Sections below Sections 1-8 below:

### 1. Antibody Affinity

In certain aspects, an antibody provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, the K_{D} between the antibody and human αvβ8 is less than about 5 nM, such as less than about 5 nM, less than about 4 nM, less than about 3 nM, less than about 2 nM, less than about 1 nM, or less than about 0.5 nM. In some embodiments, the K_{D} between the antibody and murine αvβ8 is less than about 5 nM, such as less than about 5 nM, less than about 4 nM, less than about 3 nM, less than about 2 nM, less than about 1 nM, or less than about 0.5 nM. In some embodiments, the K_{D} between the antibody and cynomolgus αvβ8 is less than about 5 nM, such as less than about 5 nM, less than about 4 nM, less than about 3 nM, less than about 2 nM, less than about 1 nM, or less than about 0.5 nM.

In one aspect, K_{D} is measured using a BIACORE® surface plasmon resonance assay. For example, an assay using a BIACORE® T200, BIACORE®-2000, a BIACORE®-3000, or a BIACORE® 8K (BIAcore, Inc., Piscataway, NJ) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensograms. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

In an alternative method, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

### 2. Antibody Fragments

In certain aspects, an antibody provided herein is an antibody fragment which specifically binds to αvβ8, such as human αvβ8, cynomolgus αvβ8, and/or murine αvβ8. An αvβ8 antibody fragment, as used herein, is any portion of an αvβ8 antibody which retains specific binding for αvβ8.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 3. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Nat'l. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 5. Library-Derived Antibodies

In certain aspects, an antibody provided herein is derived from a library. Antibodies of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, N J, 2001) and in Marks and Bradbury in Methods in Molecular Biology 248:161-175 (Lo, ed., Human Press, Totowa, N J, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

### 6. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for αvβ8 and the other specificity is for any other antigen. In certain aspects, bispecific antibodies may bind to two (or more) different epitopes of αvβ8. Multispecific (e.g., bispecific) antibodies may also be used to localize cytotoxic agents or cells to cells which express αvβ8. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to αvβ8 as well as another different antigen, or two different epitopes of αvβ8 (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for αvβ8 and the other is for CD3.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 7. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution. Insertion, and Deletion Variants

In certain aspects, antibody variants having one or more amino acid substitutions are provided. Sites of interest for substitutional mutagenesis include the CDRs and FRs. Conservative substitutions are shown in Table 6 under the heading of “preferred substitutions”. More substantial changes are provided in Table 6 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 6

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### e) Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 8. Immunoconjugates

The invention also provides immunoconjugates comprising an anti-αvβ8 antibody herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### B. Exemplary Anti-PD-L1 Antibodies

In some embodiments, the anti-αvβ8 antibody or antigen-binding fragment thereof is administered in combination with an effective amount of one or more additional therapeutic agents. In a preferred embodiment, the anti-αvβ8 antibody is administered in combination with a PD-1 axis antagonist, such as a PD-1 binding antagonist or a PD-L1 binding antagonist, such as an anti-PD-1 antibody or an anti-PD-L1 antibody. In a further preferred embodiment, the anti-αvβ8 antibody is administered in combination with an anti-PD-L1 antibody, wherein the anti-PD-L1 antibody is atezolizumab. In some embodiments, the anti-PD-L1 antibody embodiments comprises HVR-H1, HVR-H2, HVR-H3, HVR-L1, HVR-L2, and HVR-H3 as defined in Table 7, below. In some embodiments, the anti-PD-L1 antibody comprises the heavy chain variable region (VH) sequence of SEQ ID NO:216 and the light chain variable region (VL) sequence of SEQ ID NO:217. In some embodiments, the anti-PD-L1 antibody comprises the heavy chain sequence of SEQ ID NO:218 and the light chain sequence of SEQ ID NO:219. Therapeutic methods utilizing the anti-αvβ8 antibodies described herein in combination with an effective amount of one or more additional therapeutic agents are discussed in Section II(G) of this description.

TABLE 7
Exemplary anti-PD-L1 antibody sequences.

| | | |
|---|---|---|
| SEQ ID NO: 210 | Anti-PD-L1 HVR-H1 | GFTFSDSWIH |
| SEQ ID NO: 211 | Anti-PD-L1 HVR-H2 | AWISPYGGSTYYADSVKG |
| SEQ ID NO: 212 | Anti-PD-L1 HVR-H3 | RHWPGGFDY |
| SEQ ID NO: 213 | Anti-PD-L1 HVR-L1 | RASQDVSTAVA |
| SEQ ID NO: 214 | Anti-PD-L1 HVR-L2 | SASFLYS |
| SEQ ID NO: 215 | Anti-PD-L1 HVR-L3 | QQYLYHPAT |
| SEQ ID NO: 216 | Anti-PD-L1 heavy chain | EVQLVESGGGLVQPGGSLRLSCAASGFTFSDSWIHW |
| | variable region (VH) | VRQAPGKGLEWVAWISPYGGSTYYADSVKGRFTISA |
| | | DTSKNTAYLQMNSLRAEDTAVYYCARRHWPGGFDY |
| | | WGQGTLVTVSS |
| SEQ ID NO: 217 | Anti-PD-L1 light chain | DIQMTQSPSSLSASVGDRVTITCRASQDVSTAVAWYQ |
| | variable region (VL) | QKPGKAPKLLIYSASFLYSGVPSRFSGSGSGTDFTLT |
| | | ISSLQPEDFATYYCQQYLYHPATFGQGTKVEIKR |
| SEQ ID NO: 218 | Anti-PD-L1 heavy chain | EVQLVESGGGLVQPGGSLRLSCAASGFTFSDSWIHW |
| | | VRQAPGKGLEWVAWISPYGGSTYYADSVKGRFTISA |
| | | DTSKNTAYLQMNSLRAEDTAVYYCARRHWPGGFDYW |
| | | GQGTLVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKV |
| | | EPKSCDKTHTCPPCPAPELLGGPSVFLFPPKPKDTL |
| | | MISRTPEVTCVVVDVSHEDPEVKFNWYVDGVEVHNA |
| | | KTKPREEQYASTYRVVSVLTVLHQDWLNGKEYKCKV |
| | | SNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHEA |
| | | LHNHYTQKSLSLSPG |
| SEQ ID NO: 219 | Anti-PD-L1 light chain | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQYLYHPATFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |

### C. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody as used in the methods as reported herein are provided.

In one aspect, a method of making an anti-αvβ8 antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an anti-αvβ8 antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### D. Assays

Anti-αvβ8 antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with another antibodies for binding to αvβ8. In certain aspects, such a competing antibody binds to the same epitope (e.g., a linear or a conformational epitope) that is bound by the other antibody specific for αvβ8. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized αvβ8 is incubated in a solution comprising a first labeled antibody that binds to αvβ8, such as an antibody described herein, and a second unlabeled antibody that is being tested for its ability to compete with the first antibody for binding to αvβ8. The second antibody may be present in a hybridoma supernatant. As a control, immobilized αvβ8 is incubated in a solution comprising the first labeled antibody but not the second unlabeled antibody. After incubation under conditions permissive for binding of the first antibody to αvβ8, excess unbound antibody is removed, and the amount of label associated with immobilized αvβ8 is measured. If the amount of label associated with immobilized αvβ8 is substantially reduced in the test sample relative to the control sample, then that indicates that the second antibody is competing with the first antibody for binding to αvβ8. See Harlow and Lane (1988) Antibodies: A Laboratory Manual ch.14 (Cold Spring Harbor Laboratory, Cold Spring Harbor, NY).

In another aspect, the anti-αvβ8 antibodies described herein, in some embodiments, do not depend on the presence of divalent cations to bind αvβ8. In an exemplary method, binding of the anti-αvβ8 to αvβ8 (such as human αvβ8 or murine αvβ8) is assessed by SPR (such as on a BIACORE® system) in the presence and absence of divalent cations (such as MgCl₂ or CaCl₂)).

### 2. Activity Assays

In one aspect, assays are provided for identifying anti-αvβ8 antibodies thereof having biological activity. Biological activity may include, e.g., anti-tumor activity. Antibodies having such biological activity in vivo and/or in vitro are also provided.

In certain aspects, an antibody of the invention is tested for such biological activity.

In some embodiments, the anti-αvβ8 antibodies described herein inhibit αvβ8-mediated activation of L-TGFβ1 by human-leucine-rich-repeat-containing protein 33 (LRRC33) and/or latent TGFβ-binding proteins (LTBPs). Pro-TGFβ1 dimerizes and disulfide links to LTBP or GARP in large latent complexes. Binding of αvβ8 to motifs in the arm domains of pro-TGFβ1 is required for TGFβ1 activation in vivo.

### E. Methods and Compositions for Diagnostics and Detection

In certain aspects, any of the anti-αvβ8 antibodies provided herein is useful for detecting the presence of αvβ8 in a biological sample. The term “detecting” as used herein encompasses quantitative or qualitative detection. In certain aspects, a biological sample comprises a cell or tissue.

In one aspect, an anti-αvβ8 antibody for use in a method of diagnosis or detection is provided. In a further aspect, a method of detecting the presence of αvβ8 in a biological sample is provided. In certain aspects, the method comprises contacting the biological sample with an anti-αvβ8 antibody as described herein under conditions permissive for binding of the anti-αvβ8 antibody to αvβ8, and detecting whether a complex is formed between the anti-αvβ8 antibody and αvβ8. Such method may be an in vitro or in vivo method. In one aspect, an anti-αvβ8 antibody is used to select subjects eligible for therapy with an anti-αvβ8 antibody, e.g., where αvβ8 is a biomarker for selection of patients.

In certain aspects, labeled anti-αvβ8 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Compositions

In a further aspect, provided are pharmaceutical compositions comprising any of the antibodies provided herein, e.g., for use in any of the below therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions (formulations) of an anti-αvβ8 antibody as described herein can be prepared by combining the antibody with pharmaceutically acceptable carriers or excipients known to the skilled person. See, for example Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1^{st} Ed., Woodhead Publishing (2015), § 4 and Falconer R. J., Biotechnology Advances (2019), 37, 107412. Exemplary pharmaceutical compositions of an anti-αvβ8 antibody as described herein are lyophilized, aqueous, frozen, etc.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### G. Therapeutic Methods and Routes of Administration

Any of the anti-αvβ8 antibodies provided herein may be used in therapeutic methods.

In one aspect, an anti-αvβ8 antibody for use as a medicament is provided. In further aspects, an anti-αvβ8 antibody for use in treating cancer, including, but not limited to, glioblastoma multiforme (GBM), low-grade glioma, pheochromocytoma, adrenal cancer, ovarian cancer, melanoma, uveal melanoma, sarcoma, mesothelioma, clear cell renal cell carcinoma (ccRCC), thymoma, papillary RCC, germ cell cancer, diffuse large B-cell lymphoma (DLBCL), breast cancer, such as triple-negative breast cancer (TNBC), non-small cell lung cancer (NSCLC), colorectal cancer, cholangiocarcinoma, endometrial cancer, kidney renal papillary cancer, or bladder cancer, is provided. In some embodiments, the cancer is a cancer exhibiting increased expression of αvβ8 and reduced expression of αvβ6, such as compared with normal tissue or compared to a cancer of the same type. For example, in some embodiments, after comparing two cancers of the same type (such as two breast cancers), the cancer having the higher expression of αvβ8, lower expression of αvβ6, and/or a higher ratio of αvβ8 to αvβ6 is selected for treatment with the anti-αvβ8 antibody.

In certain aspects, an anti-αvβ8 antibody for use in a method of treatment is provided. In certain aspects, the invention provides an anti-αvβ8 antibody for use in a method of treating an individual having a cancer, including, but not limited to, ovarian cancer, triple-negative breast cancer (TNBC), non-small cell lung cancer (NSCLC), colorectal cancer, cholangiocarcinoma, endometrial cancer, kidney renal papillary cancer, or bladder cancer, the method comprising administering to the individual an effective amount of the anti-αvβ8 antibody. In one such aspect, the method further comprises administering to the individual an effective amount of at least one additional therapeutic agent (e.g., one, two, three, four, five, or six additional therapeutic agents), e.g., as described below. An “individual” according to any of the above aspects is preferably a human.

In a further aspect, the invention provides for the use of an anti-αvβ8 antibody in the manufacture or preparation of a medicament. In one aspect, the medicament is for treatment of cancer, such as glioblastoma multiforme (GBM), low-grade glioma, pheochromocytoma, adrenal cancer, ovarian cancer, melanoma, uveal melanoma, sarcoma, mesothelioma, clear cell renal cell carcinoma (ccRCC), thymoma, papillary RCC, germ cell cancer, diffuse large B-cell lymphoma (DLBCL), breast cancer, such as triple-negative breast cancer (TNBC), non-small cell lung cancer (NSCLC), colorectal cancer, cholangiocarcinoma, endometrial cancer, kidney renal papillary cancer, or bladder cancer, the method comprising administering to an individual having cancer an effective amount of the medicament. In one such aspect, the method further comprises administering to the individual an effective amount of at least one additional therapeutic agent, e.g., as described below. An “individual” according to any of the above aspects may be a human.

In a further aspect, the invention provides a method for treating cancer, such as glioblastoma multiforme (GBM), low-grade glioma, pheochromocytoma, adrenal cancer, ovarian cancer, melanoma, uveal melanoma, sarcoma, mesothelioma, clear cell renal cell carcinoma (ccRCC), thymoma, papillary RCC, germ cell cancer, diffuse large B-cell lymphoma (DLBCL), breast cancer, such as triple-negative breast cancer (TNBC), non-small cell lung cancer (NSCLC), colorectal cancer, cholangiocarcinoma, endometrial cancer, kidney renal papillary cancer, or bladder cancer. In one aspect, the method comprises administering to an individual having said cancer an effective amount of an anti-αvβ8 antibody. In some embodiments, the cancer is ovarian cancer. In some embodiments, the cancer is triple-negative breast cancer (TNBC). In some embodiments, the cancer is non-small cell lung cancer (NSCLC). In some embodiments, the cancer is colorectal cancer. In some embodiments, the cancer is cholangiocarcinoma. In some embodiments, the cancer is endometrial cancer. In some embodiments, the cancer is kidney renal papillary cancer. In some embodiments, the cancer is bladder cancer. In one such aspect, the method further comprises administering to the individual an effective amount of at least one additional therapeutic agent, as described below.

An “individual” according to any of the above aspects may be a human of any age, such as an adult human.

In a further aspect, the invention provides pharmaceutical compositions comprising any of the anti-αvβ8 antibodies provided herein, e.g., for use in any of the above therapeutic methods. In one aspect, a pharmaceutical composition comprises any of the anti-αvβ8 antibodies provided herein and a pharmaceutically acceptable carrier. In another aspect, a pharmaceutical composition comprises any of the anti-αvβ8 antibodies provided herein and at least one additional therapeutic agent, e.g., as described below.

Antibodies of the invention can be administered alone or used in a combination therapy. For instance, the combination therapy includes administering an antibody of the invention and administering at least one additional therapeutic agent (e.g. one, two, three, four, five, or six additional therapeutic agents). In certain aspects, the combination therapy comprises administering an antibody of the invention and administering at least one additional therapeutic agent, such as a PD-1 axis antagonist, such as a PD-1 binding antagonist or a PD-L1 binding antagonist, such as an anti-PD-1 antibody or anti-PD-L1 antibody. In a preferred embodiment, the PD-1 axis antagonist is an anti-PD-L1 antibody, preferably atezolizumab.

The additional therapeutic agent, such as the PD-1 axis antagonist, may be administered before, concurrently, or after administration of the anti-αvβ8 antibody. As used herein, “concurrently” does not necessarily mean the anti-αvβ8 antibody and the additional therapeutic agent are present in the same composition. It is understood that administration of the anti-αvβ8 antibody and the additional therapeutic agent at a similar time, such as on the same day, would be a concurrent administration. Before or after administration of the anti-αvβ8 may be at least 1 day before or after, such as at least 1 day, at least 2 days, at least 3 days, at least 4 days, at least 5 days, at least 6 days, at least a week, at least two weeks, at least three weeks, at least a month, or more before or after administration of the anti-αvβ8 antibody.

It is understand that the anti-αvβ8 antibody and the additional therapeutic agent or agents in the combination therapy may have different dosing schedules. Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents, such as a PD-1 axis antagonist, such as atezolizumab. In one aspect, administration of the anti-αvβ8 antibody and administration of an additional therapeutic agent, such as a PD-1 axis antagonist, occur within about one month, or within about one, two or three weeks, or within about one, two, three, four, five, or six days, of each other. In one aspect, the antibody and additional therapeutic agent are administered to the patient on Day 1 of the treatment. Antibodies of the invention can also be used in combination with radiation therapy.

An antibody of the invention (an anti-αvβ8 antibody), and any additional therapeutic agent, can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. For repeated administrations over several days or longer, depending on the condition, the treatment would generally be sustained until a desired suppression of disease symptoms occurs. However, other dosage regimens may be useful. The progress of this therapy is easily monitored by conventional techniques and assays.

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. Examples

The following are examples of methods and compositions of the invention. It is understood that various other embodiments may be practiced, given the general description provided above.

### Example 1: Generation of Rabbit Anti-αvβ8 Monoclonal Antibodies

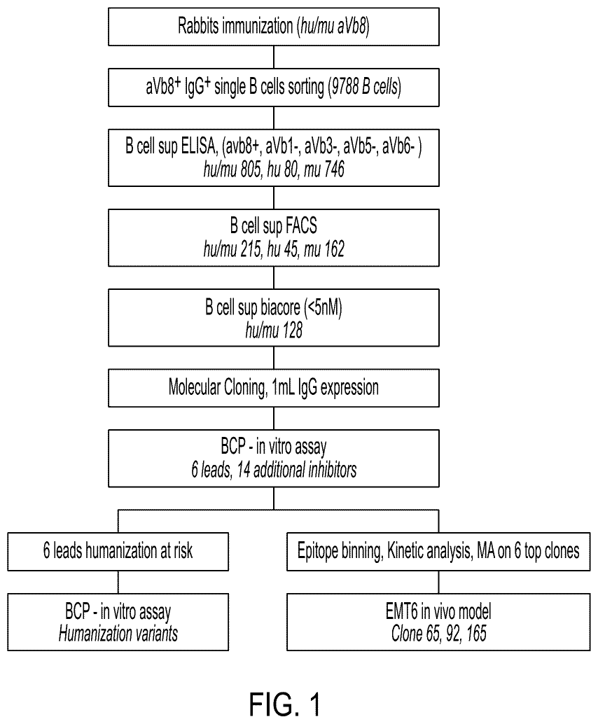

αvβ8 antibody screening workflow was performed as shown in the schematic of FIG. 1. New Zealand White (NZW) rabbits were co-immunized with human αvβ8 recombinant protein, and murine αvβ8 (PUR1BX42109) made in house. Single B cells were isolated using a protocol related to published literature. See, e.g., Lin et al., Rapid identification of anti-idiotypic mAbs with high affinity and diverse epitopes by rabbit single B-cell sorting-culture and cloning technology, PLoS ONE 15(12), 2020. This workflow included direct FACS sorting of IgG+, human and/or murine αvβ8+B cells (total of 9788 B cells) into single wells. The B cells were cultured for 7 days and the supernatants were assayed by ELISA, FACS and SPR for binding to both human and murine αvβ8. For the ELISA screening, the supernatants were assayed for binding to both human and murine αvβ8, and countered screened against other human integrins including αvβ1, 3, 5, and 6. 805 B cells bound to both human and murine αvβ8 proteins, in addition to 45 human-specific and 162 murine-specific B cells. ELISA positive B cells were lysed and immediately frozen in −80° C. for storage until molecular cloning. ELISA hu/mu double positive B cell supernatants were subsequently screened by FACS analysis for cell binding to LN229 (human cells endogenously expressing αvβ8) and TRAMPC2-H1 (murine cells endogenously expressing αvβ8). FACS analysis further narrowed down 215 B cell clones.

The supernatants for these 215 B cells were further screened for affinity by SPR binding with a BIACORE® 8K machine. Briefly, the rabbit B cell supernatants were first captured on a protein A sensor chip. Both human and murine αvβ8 solutions at 100 nM concentration were then injected through the flow cells. Clones that bind equally to both human and murine with an apparent K_{D} of 5 nM or less were selected for molecular cloning. Variable regions (VH and VL) of each monoclonal antibody from rabbit B cells were cloned into expression vectors from extracted mRNA as previously described. Individual recombinant rabbit antibodies were expressed in Expi293 cells and subsequently purified with protein A.

Sprague Dawley rats (Charles River, Hollister, CA) were immunized with human and mouse αvβ8 recombinant proteins solubilized in CFA (Sigma-Aldrich, St. Louis, MO) or detergent mixed with MPL+TDM adjuvant (Sigma-Aldrich, St. Louis, MO) or mixed with a combination of TLR agonists: 50 μg MPL (Sigma-Aldrich), 20 μg R848 (Invivogen, San Diego, CA), 10 μg PolyJ:C (Invivogen), and 10 ug CpG (Invivogen) divided among multiple sites. Additional 6-8 boosts were given bi-weekly. After immunization, enriched B cells from lymph nodes were FACS sorted and single avb8-positive cells were cultured as described previously (Marei H et al, Nature 610 (7930): 182-9 (2022)). A total of 10,752 single B cells were sorted, and supernatants were analyzed for ELISA binding against human and murine avb8 recombinant proteins (154 clones positive) or cell lines expressing human or murine avb8 (75 clones positive). RNA was extracted from B-cells that showed FACS binding for molecular cloning and recombinant expression.

For immunizations, full-length ectodomains (headpiece and leg) were used for both αV (residues M1 to V992) and β8 (residues M1 to R684). Heterodimerization was induced absent the transmembrane domains of αV and β8 by fusing an acid/basic coil to the C-terminus of αV and β8 ECDs, respectively. αvβ8 positive (αvβ8+) immunoglobulin G positive (IgG+) single B cells were isolated. Hu αvβ8+/IgG+ B cells were fused with LN229 cells and mu αvβ8+/IgG+ B cells were fused with TRAMPC2-H1 cells. The B cell supernatant was then collected and screened for antigen-specific (αvβ8+, αVβ1−, αVP3−, αVP5−, αVP6−) antibodies via ELISA, FACs, and BIACORE®.

Purified anti-αvβ8 antibodies were screened for binding affinity to αvβ8, selectivity to αvβ8, epitope characterization and cell-based functional activity.

αvβ1 Binding Assay with Rabbit αvβ8 Antibody

The binding affinity of the antibodies was determined by BIAcore™ T200 machine. For kinetics measurements, antibodies were captured on research grade protein A chip (Cytiva, USA) to achieve approximately 60 RU. Ten-fold serial dilutions of human, murine and cyno aVb8 were injected in the same running buffer as above at 37° C. with a flow rate of 100 μL/min. Association rates (ka) and dissociation rates (kd) were calculated using a 1:1 Langmuir binding model (BIAcore™ T200 Evaluation Software version 2.0). The equilibrium dissociation constant (KD) was calculated as the ratio kd/ka. No binding was observed to human αVP1, αV03, αV05, or αV06, confirming that these antibodies are specific.

The ability of rabbit αvβ8 antibodies to block αvβ8-dependent activation of L-TGFβ1 was determined. LN-229 cells expressing αvβ1 were co-cultured with 3T3-Nano luciferase TGFβ C reporter cells, which express cell surface molecule GARP (that binds TGFβ1) and human TGFβ1.

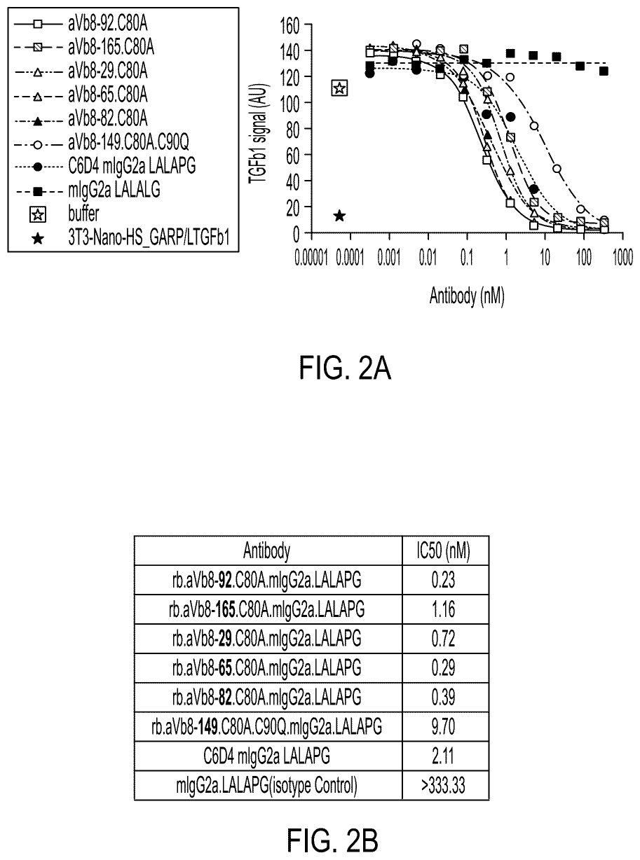

FIG. 2A and FIG. 2B shows the IC50 (nM) values of the rabbit αvβ8 antibodies. The rabbit αvβ8 antibodies had lower IC50 values compared to mAb C6D4.

Cross-Reactivity of Rabbit αvβ8 Antibody with Cynomolgus, Human or Murine αvβ8

An array-based SPR imaging system (Carterra USA) was used to epitope bin the 20 most potent rabbit monoclonal antibodies, including ADWA11-2.4 and C6D4. Purified antibodies were diluted at 10 ug/ml in 10 mM sodium acetate buffer pH 4.5. Using amine coupling, antibodies were directly immobilized onto a SPR sensorprism CMD 200M chip (XanTec Bioanalytics, Germany) using a Continuous Flow Microspotter (Carterra, USA). For analysis, the IBIS MX96 SPRi (Carterra USA) was used to evaluate analytes binding to the immobilized ligands. Human αvβ8 was first injected for 4 minutes at 50 nM and was followed by a second 4 minutes injection of individual monoclonal antibody at 10 ug/ml. The surface was regenerated with 10 mM glycine pH1.5 between cycles. The experiment was performed at 25° C. in a running buffer of 0.01M HEPES pH 7.4, 0.15M NaCl, 0.05% surfactant P20, 0.5 mM CaCl₂). The epitope binning data was processed using Carterra binning software tool.

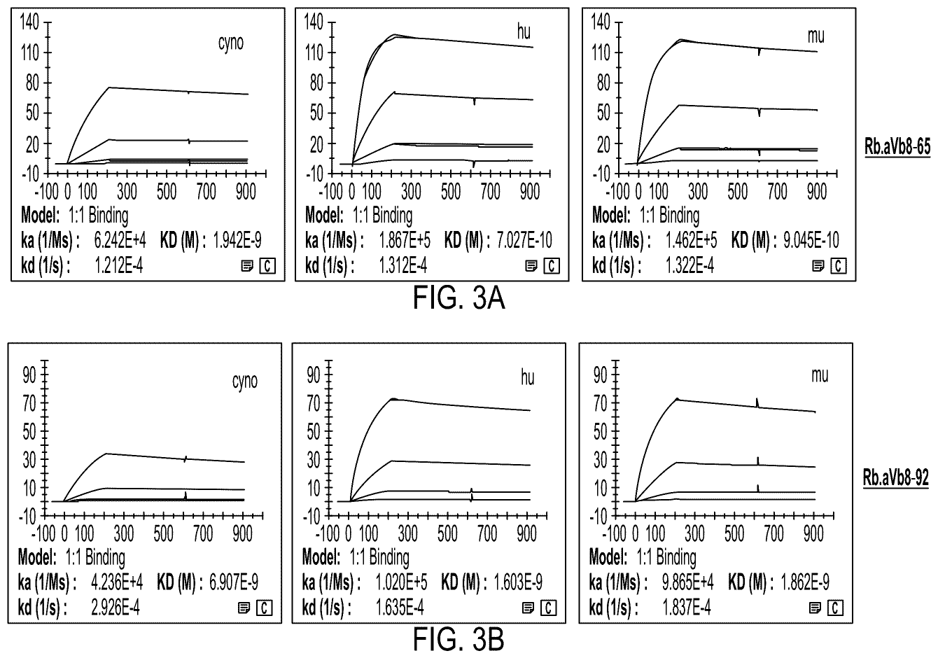

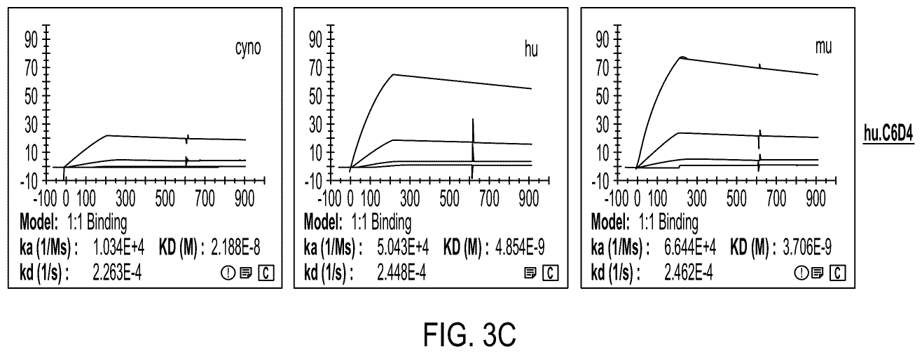

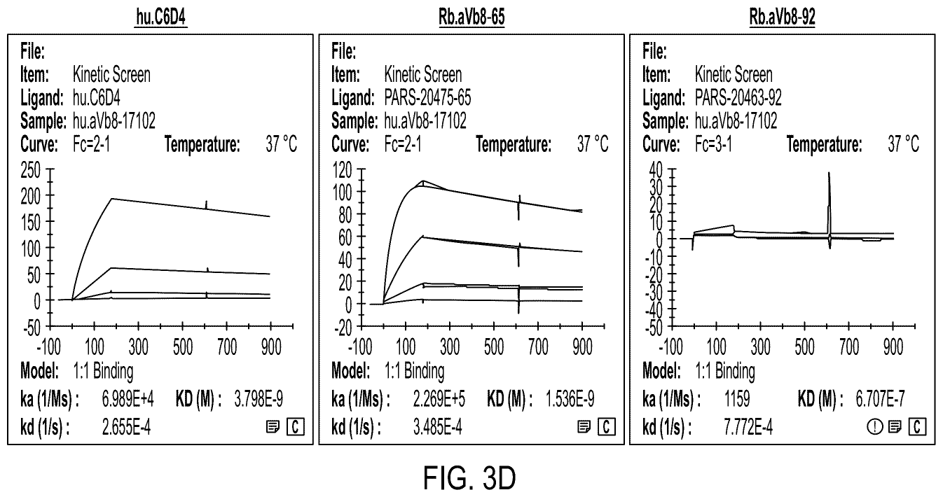

Relative binding of the rabbit αvβ8 antibodies to cynomolgus, human, or murine αvβ8 was determined. The binding assay was performed by SPR. The results of three separate binding assays are shown in Table 8 and FIGS. 3A-3C. Relative binding of commercial human C6D4 mIgG2a LALALG control antibody and rabbit αvβ8 antibodies was assessed in the presence of divalent cations (FIG. 3D).

TABLE 8
rabbit αvβ8 antibody binding assay

| | hu.aVb8.17102 | | | mu.aVb8.17103 | | | cyno.aVb8.13650 | | |
|---|---|---|---|---|---|---|---|---|---|
| | | | KD | | | KD | | | KD |
| Ligand | ka (1/Ms) | kd (1/s) | (nM) | ka (1/Ms) | kd (1/s) | (nM) | ka (1/Ms) | kd (1/s) | (nM) |
| rb.aVb8-65 | 1.87E+05 | 1.31E−04 | 0.70 | 1.46E+05 | 1.32E−04 | 0.9 | 6.24E+04 | 1.21E−04 | 1.94 |
| rb.aVb8-92 | 1.02E+05 | 1.64E−04 | 1.60 | 9.86E+04 | 1.84E−04 | 1.86 | 4.24E+04 | 2.93E−04 | 6.91 |
| hC6D4 | 5.04E+04 | 2.45E−04 | 4.85 | 6.64E+04 | 2.46E−04 | 3.71 | 1.03E+04 | 2.26E−04 | 2.19 |

Rabbit αvβ8 antibodies rb.αvβ8-65 and rb.αvβ8-92 bind human and murine αvβ8 as well as commercial αvβ8 antibody huC6D4 (FIG. 3A-3C).

### Example 2: Humanization of Rabbit αvβ8 Antibodies

After antibody screening as described in Example 1, monoclonal antibodies with inhibitory activities were chosen for further characterization. The antibodies designated αvβ8-65 and αvβ8-92, which had the strongest binding affinity, inhibitory activity, and fewest manufacturing issues, were selected for humanization.

Rabbit monoclonal antibodies αvβ8-65 and αvβ8-92 were humanized as described below. Residue numbers are according to Kabat et al., Sequences of proteins of immunological interest, 5th Ed., Public Health Service, National Institutes of Health, Bethesda, Md. (1991).

Variants constructed during the humanization of rb.αvβ8-65 and rb.αvβ8-92 were assessed in the form of human IgG. Hypervariable regions from each of the rabbit antibodies (namely positions 24-34 (L1), 50-56 (L2) and 89-97 (L3) in VL domain, and 26-35 (H1), 50-65 (H2) and 95-102 (H3) in VH domain) were grafted into various acceptor frameworks. Specifically, for αvβ8-65, VL CDRs were grafted into KV1-12*01, and VH CDRs were grafted into HV3-23*01. All VL and VH Vernier positions from rabbit antibodies were also grafted into their respective human germline frameworks. The grafts with all rabbit amino acids in Vernier positions are referred to as H1L1 (hu.αvβ8-65.H1L1).

The binding affinity of hu.αvβ8.H1L1 antibody was compared to their chimeric parental clones. Rabbit Vernier positions of version H1L1 antibodies were converted back to human residues to evaluate the contribution of each rabbit Vernier positions to binding affinity to human αvβ8. One additional light chain variant L2 (CDR graft), and ten additional heavy chain variants H2-H11 were made. Gly49 on the heavy chain was determined to be the key rabbit Vernier residues based on binding affinity evaluation of the variant antibodies described above (data not shown). The G49 together with CDR residues were also grafted onto human germline HV3-23*01 as H15. The final humanized sequence for αvβ8-65 is hu.αvβ8-65.H15L2.

For rb.αvβ8-92, VL CDRs were grafted into KV4-1*01, and VH CDRs were grafted into both HV3-33*02 HV3-23*02. All VL and VH Vernier positions from rabbit antibodies were also grafted into their respective human germline frameworks. The grafts with all rabbit amino acids in Vernier positions are referred to as H1L1 (hu.αvβ8-92.H1L1). Similar to above described for αvβ8-65, all VL and VH Vernier positions from rabbit antibodies were also grafted into their respective human germline frameworks. Nine additional heavy chain variants H2-H10 were made for HV3-33: H2-10. For light chain, all three Vernier residues Ala2, Leu4, and Arg68 were determined to be the key rabbit residues based on binding affinity evaluation of the variant antibodies described above (data not shown). For heavy chain, Gln2, Ile48, Gly49, Ser73, Phe91, and Pro105 were determined to be the key rabbit residues based on binding affinity evaluation of the variant antibodies described above (data not shown). These residues together with CDR residues were also grafted onto human germline HV3-23*02 as H13. The final humanized sequence for αvβ8-92 is hu.αvβ8-92.H13L1.

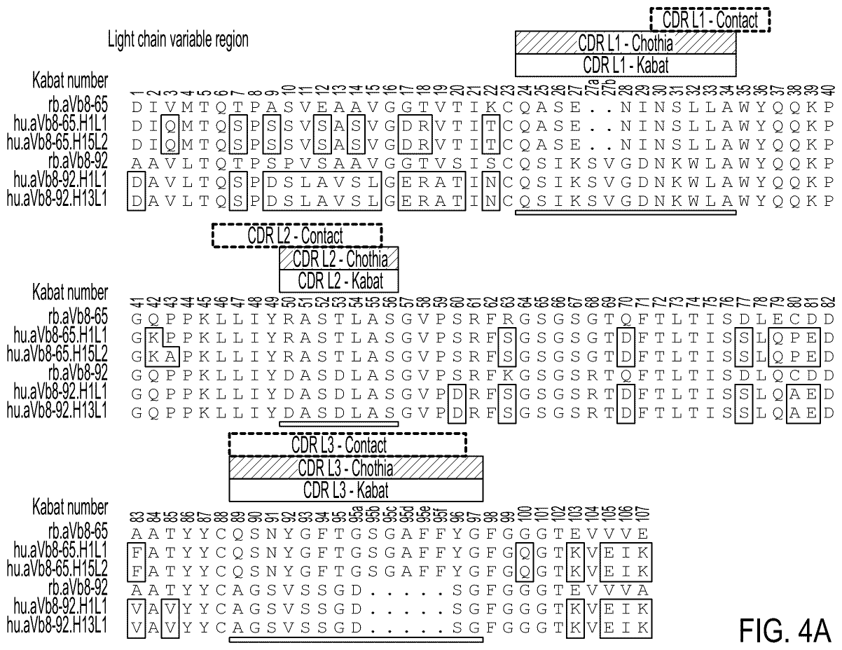

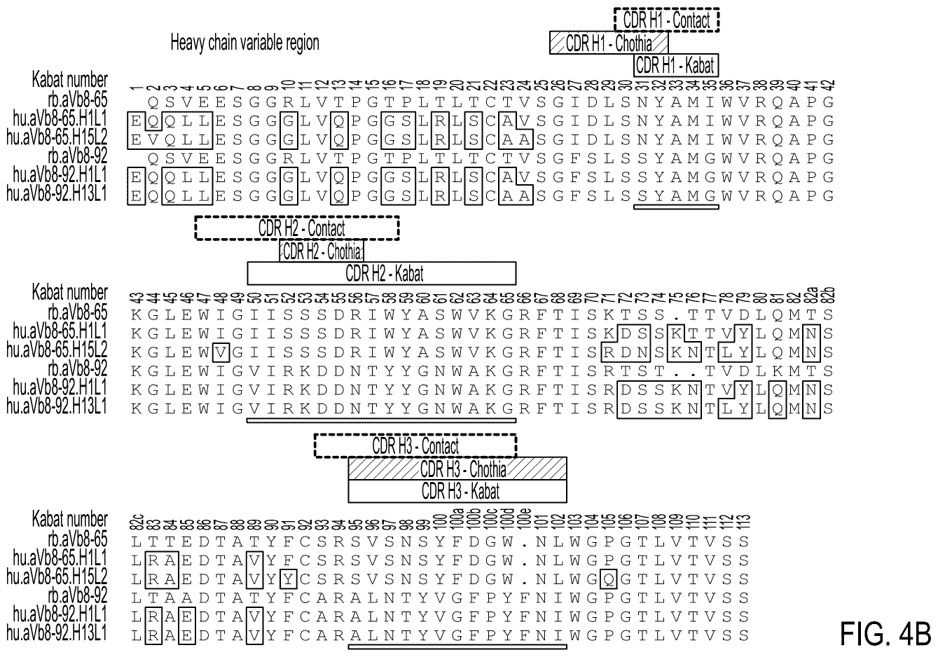

The sequences of the humanized and rabbit αvβ8 antibodies are provided in Tables 1-5 of this description and a sequence alignment of light chain variable region and heavy chain variable region sequences between the humanized and rabbit αvβ8 antibodies is provided in FIG. 4A and FIG. 4B.

Structure and interaction of rabbit anti-αvβ8-65 and αvβ8. A cryo-EM structure determination assay was performed on αvβ8 FAB to assess antibody structure. FAB fragments including heavy and light Chains for FAB 68 (Cormier NSMB 2018 PMID 30061598) were expressed in CHO cells. Protein was purified using protein G chromatography resin followed by size exclusion chromatography and dialysis. The final sample was at a concentration of 5 mg/mL in 20 mM Histidine Acetate, 0.15 M NaCl, pH5.5.

FAB fragments including heavy and light Chains for FAB 8B8 (Cormier NSMB 2018 PMID 30061598) were expressed in CHO cells. Protein was purified using protein G chromatography resin followed by size exclusion chromatography and dialysis. The final sample was at a concentration of 5.6 mg/mL in 20 mM Histidine Acetate, 0.15 M NaCl, pH5.5.

FAB fragments including heavy and light Chains for FAB 65 (anti-ITGB8.αvβ8-65) were expressed in Expi293 cells. Protein was purified using protein G chromatography resin followed by size exclusion chromatography and dialysis. The final sample was at 3.47 mg/mL in 20 mM Sodium Acetate, 150 mM NaCl, pH4.5.

Recombinant human αvβ8 was obtained by co-expressing residues M1-V992 of human Integrin Subunit Alpha (αV) and residues M1-R684 of human Integrin Subunit Beta 8 (b8) in HEK293 cells. To facilitate formation and purification of the αvβ8 heterodimer, an acid coil and Strep Tag® were fused to the C-terminus of the αV domain, and a basic coil and hexa-histidine tag were fused to the C-terminus of the b8 domain. The protein was purified using immobilized metal affinity chromatography followed by size exclusion chromatography. The final protein concentration was at 2.6 mg/mL in 20 mM Histidine Acetate, 0.15 M NaCl, 1 mM CaCl₂), 1 mM MgCl2, pH5.5.

Purified αvβ8+FAB 65+FAB 68+FAB 8B8 complex (4 μL) was applied to glow-discharged grids with a holey gold film (UltraAuFoil®, Protochips; Morrisville, NC) and plunge frozen in liquid ethane using a Vitrobot™ (Thermo Fisher Scientific) vitrification robot. A set of 16,093 movie stacks was collected using a Titan Krios equipped with a Falcon4 detector (pixel size of 0.731 Å; Thermo Fisher Scientific; Waltham, MA). Images were processed using the software packages cryoSPARC™ Live, cryoSPARC™ (Structura Biotechnology; Toronto, ON), and cisTEM (Grant et al. 2018), yielding a final three dimensional map with an estimated resolution of 2.45 Å.

The Protein Data Bank (PDB) coordinates of the already published structure of αvβ8 (PDB code 6UJB) were docked into the map, as were the initial models of the Fabs generated using Swiss Model. Coordinates were then refined by iterative interactive rebuilding using the crystallographic object oriented toolkit (PMID 20383002) and real space refinement (PMID 31588918).

FAB 68 and FAB 8B8 (Cormier NSMB 2018 PMID 30061598) were included in the sample preparation to act as structural chaperones and facilitate high-resolution structure determination using cryo-EM. Neither FAB 68 nor FAB 8B8 alter αvβ8 function (Cormier NSMB 2018 PMID 30061598). The αvβ8+FAB 65+FAB 68+FAB 8B8 complex was formed by mixing 60 uL αvβ8 with 30 uL of FAB 65, 20 uL of FAB 68, and 20 uL FAB 8B8 and incubating on ice for 2 h. The stoichiometric complex was then isolated by pooling the appropriate peak fractions from size exclusion chromatography in 25 mM Hepes, 150 mM NaCl, 1 mM CaCl₂), 1 mM MgCl2, pH 7.2 (data not shown).

Cryo-EM analysis of the αvβ8+FAB 65+FAB 68+FAB 8B8 sample yielded a three-dimensional reconstruction of the αvβ8+FAB 65+FAB 68+FAB 8B8 complex at 2.4 Å resolution, allowing the unambiguous definition of the Fab65epitope on human αvβ8 (data not shown). Comparison of the obtained aVb8+FAB 65+FAB 68+FAB 8B8 complex structure with the published structure of human αvβ8 in complex with porcine latent transforming growth factor β1 (L-TGFb1) (PDB ID 6UJA, PMID 31955848—Campbell et al 2020 Cell 180: 490) confirmed that FAB 65 prevents L-TGFb1 binding to αvβ8 by sterically blocking its access (data not shown).

An analysis of the interaction interface between FAB 65 and αvβ8 indicates a buried surface area on αVP8 of about 1200 Å², with contributions from both the variable heavy and variable light chain of FAB 65. The CDRH2, CDRH3, CDRL1 and CDRL3 loops of FAB 65 bind at the ligand-binding cleft between αV and 08, where the RGDLXXI/L consensus motif of L-TGFb1 (also referred to as the integrin-binding motif) also binds (data not shown).

In particular, residues on the CDRH2 loop of FAB 65 bind to the specificity-determining loop 1 (SDL1) of β8 and the alpha head region of αV, interacting with similar αVP8 regions as the L-TGFb1 integrin-binding motif (data not shown). Residues from the CDRL1 and CDRL3 loops of FAB 65 form an extensive polar interaction network with the specificity-determining loop 2 (SDL2) of β8 and the alpha head region of αV (data not shown). Taken together, the interactions formed between FAB 65 and αVP8 further rationalize how FAB 65 binding to αVP8 prevents L-TGFb1 or L-TGFβ3 from binding to αVβ8.

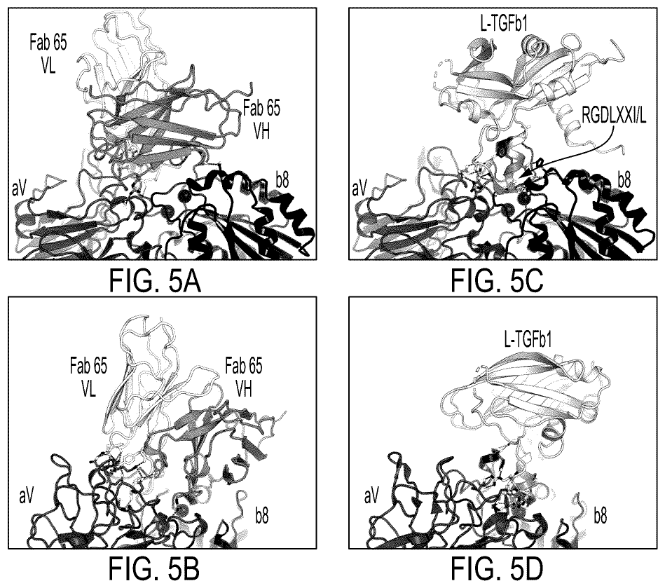

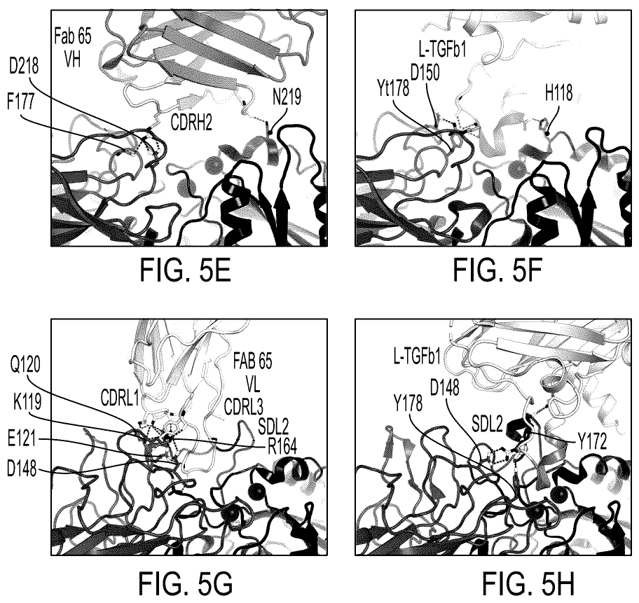

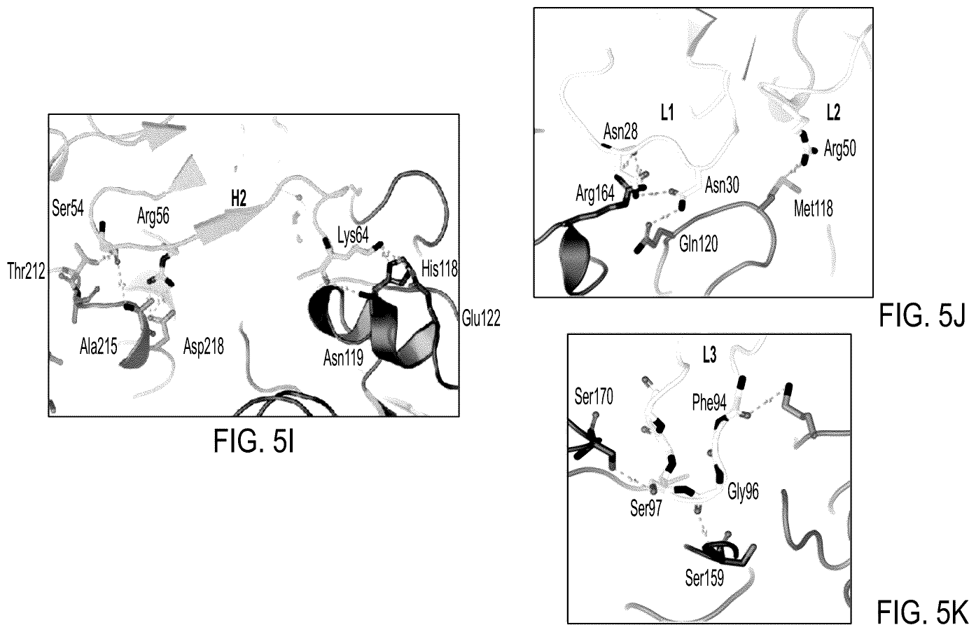

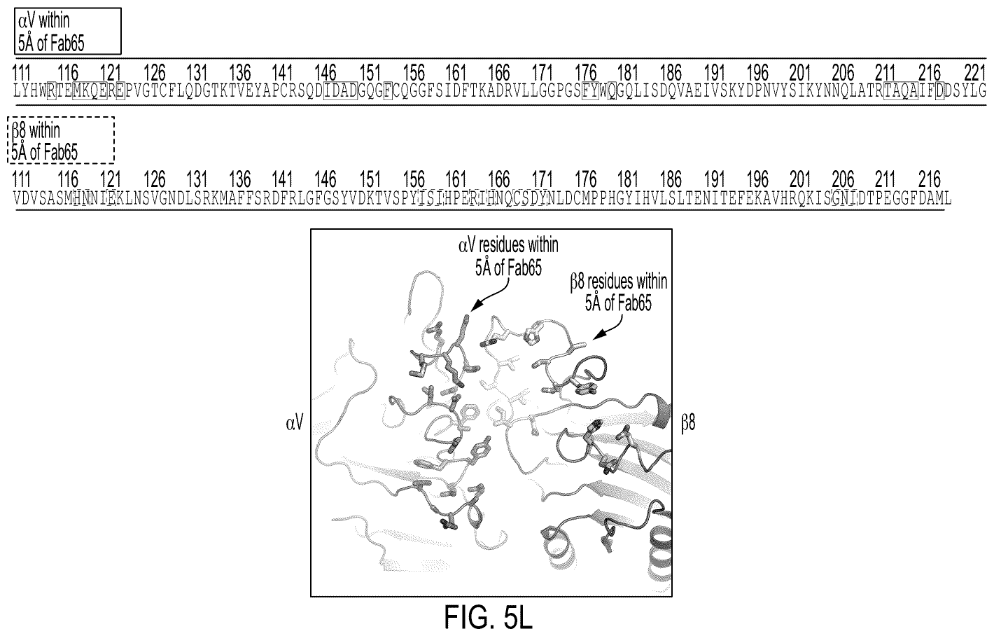

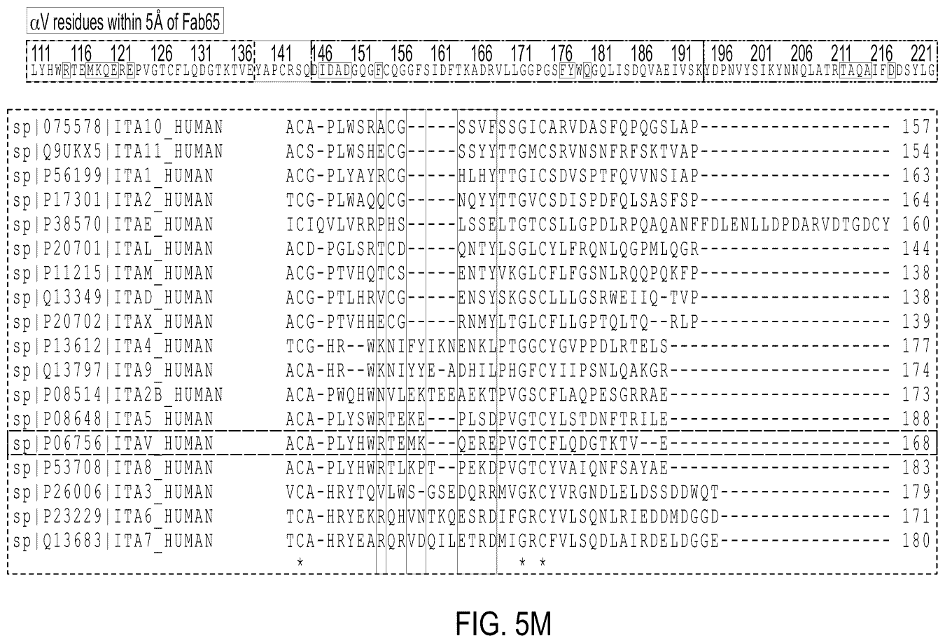

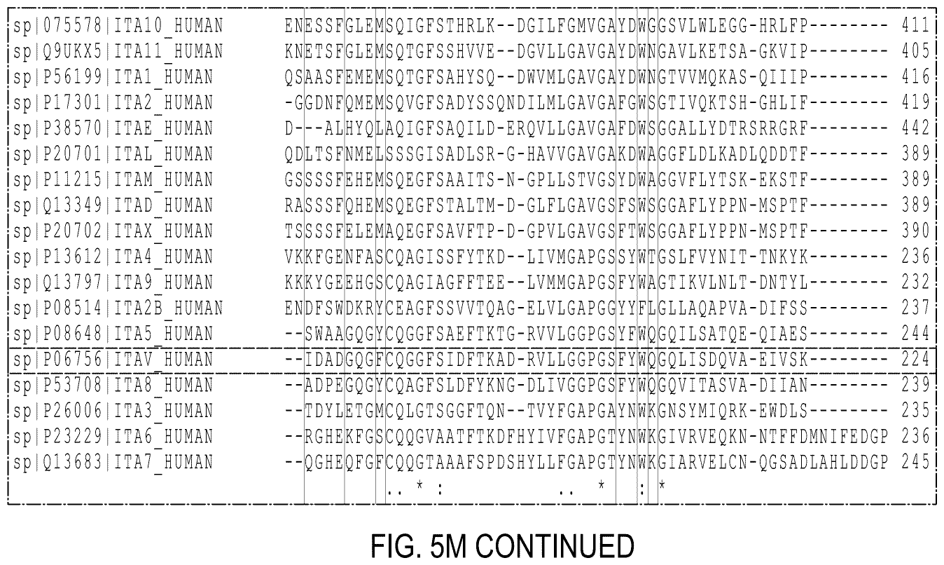

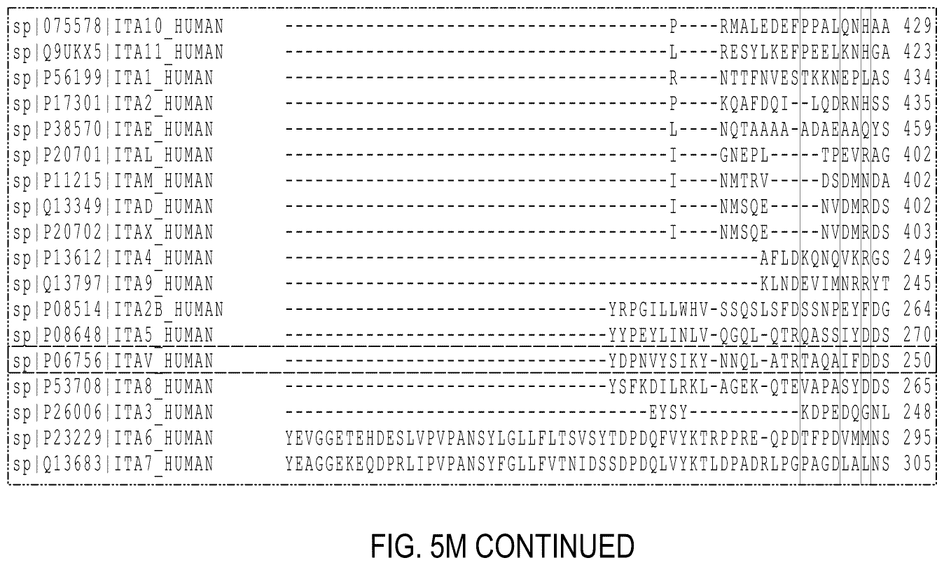

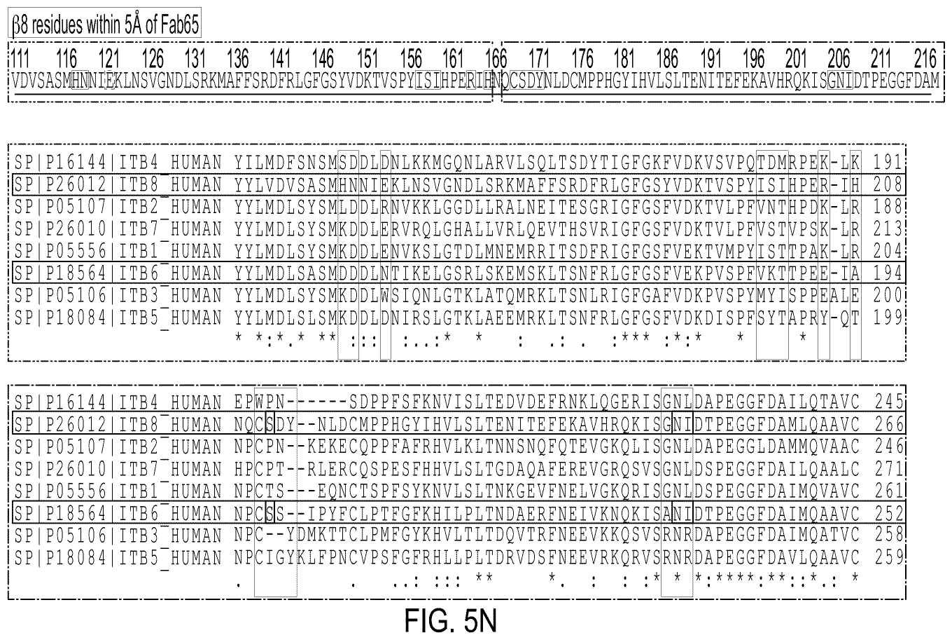

Furthermore, FIGS. 5A-5D demonstrate the CryoEM results highlighting the interaction between rb.αVP8-65 (anti-αVβ8 integrin antibody) and αVP8 integrin (FIG. 5A and FIG. 5B (rotated 90° relative to FIG. 5A)), latent-TGFβ1 (L-TGFβ1) and αVP8 integrin (FIG. 5C and FIG. 5D (rotated 90° relative to FIG. 5A)). FIGS. 5A-5D demonstrate that the binding of Fab65 blocks the interaction of latent-TGFβ1 to αVβ8. FIGS. 5C and 5D shown the position of the RGDLXXI/L motif of L-TGFb1 slotting into the interface between αV and β8 subunits. FIGS. 5A and 5B demonstrate that the FAB65 occupies a similar position as L-TGFb1 with respect to αVβ8, effectively blocking the L-TGFb1 binding site.

FIGS. 5E-5H are expanded views of the interface between hu.αVP8-65 (anti-αVβ8 integrin antibody) and αVβ8 integrin (FIGS. 5E and 5G) and the interface between L-TGFβ1 and αVβ8 integrin (FIGS. 5F and 5H). Several residues from αVβ8 integrin that interact with either hu.αVP8-65 or L-TGFβ1 are highlighted, respectively. FIGS. 5E and 5G demonstrate that residues F177 and D218 of αV make specific contacts with CDRH2; K119, Q120, E121 and D148 of αV make specific contacts with CDRL1; N219 of β8 makes specific contacts with CDRH2; and R164 of β8 makes specific contacts with CDRL1

FIGS. 5I-5K are expanded views of the interfaces between hu.αVP8-65 (anti-αVβ8 integrin antibody) and αVβ8 integrin showing salt bridges formed between the hu.αVP8-65 (anti-αVβ8 integrin antibody) and αVβ8 integrin. Particular residues are highlighted.

FIG. 5L shows the sequence of the αV and β8 subunits of the αVβ8 and an EM structure of the interface between hu.αVP8-65 (anti-αVβ8 integrin antibody) and αVβ8 integrin. Residues within 5 Å of hu.αVP8-65 are highlighted in the sequence of the αV (i.e., R115, 118M, 119K, 120Q, 121E, 123E, 1471, 148D, 149A, 150D, 154F, 177F, 178Y, 180Q, 212T, 213A, 214Q, 215A, and 218D) and β8 (i.e., 118H, 119N, 122E, 1581, 159S, 1601, 164R, 166H, 169C, 170S, 171D, 172Y, 206G, 207N, 2081) subunits of the αVβ8 and depicts the putative αVβ8 epitope as bound by Fab65.

FIG. 5M demonstrates subtype specificity assessment of binding between Fab65 and αV compared to other αV integrins.

FIG. 5N demonstrates subtype specificity assessment of binding between Fab65 and β8 compared to other β8 integrins.

The aim of this study was to define the site of interaction between human αVβ8 and the TGFb1-blocking FAB 65 antibody fragment. Size exclusion chromatography shows that aVb8 forms a 1:1 stoichiometric complex with FAB 65. Analysis of a cryo-EM structure of the αVβ8-FAB 65 complex indicates that FAB 65 binds directly to aVb8 and thereby sterically blocks binding of TGFb1 to αVP8.

Screening of rabbit and human αVP8-65 and αVP8-92 with a baculovirus (BV) binding assay. One method to reduce the number of antibodies with fast nonspecific clearance (CL) is to screen antibodies for general nonspecific binding using a baculovirus (BV) binding assay (Hötzel, I. et al., (2012) mAbs 4(6):753-760; Yadav, D B et al., (2015) J. Biol. Chem. 290:29732-29741, WO 2013/177470). Briefly, antibodies can be screened for binding baculovirus particles, lysates or antigens in an ELISA. The result is a baculovirus score (BV score). In some embodiments, an antibody is selected if uptake of the antibodies by a population of macrophages is equal to or less than a predetermined threshold and the BV score is less than about any of 1, 2, 3, 4, or 5.

Antibodies that show non-specific binding to baculovirus are more likely to have a short half-life in vivo. Rabbit and humanized αVP8-65 and αVP8-92 passed a BV ELISA test. BV test scores can be found in Table 9, below.

TABLE 9
BV ELISA Test Scores

| Clone | BV Score | Clone | BV Score |
|---|---|---|---|
| rb.65.m2a.LALAPG | 0.074 | hu.C6D4.m2a.LALAPG | 0.176 |
| hu.65.H15L2.m2a.LALAPG | 0.093 | hu.ADWA11-2.4.m2a.LALAPG | 0.276 |
| rb.92.m2a.LALAPG | 0.131 | High score control | 1.639 |
| hu.92.H13L1.m2a.LALAPG | 0.083 | Low score control | 0.342 |

Screening of humanized αVP8-65 and αVP8-92 with TGFβ reporter cells. Co cultures of LN 229 and TGFβ reporter cells overexpressing human GARP and latent TGF-β1 were used to evaluate the blocking activity of anti-αVβ8 antibodies against activation of latent TGF-β1. LN 229 cells, derived from a human glioblastoma, were maintained in high glucose Dulbecco's Modified Eagle Medium (DMEM) containing 10% fetal bovine serum (FBS; VWR; Brisbane, CA), 2 mM L glutamine, 100 units/mL penicillin, and 100 μg/mL streptomycin at 37±0.5° C. with 5% CO₂. TGF-β reporter cells, derived from the 3T3 fibroblast cell line, were transfected with a SMAD inducible NanoLuc® luciferase reporter gene and a constitutively expressed firefly luciferase gene. Further stable transfections were performed with human GARP and latent TGF-01. The cells were maintained in high glucose DMEM with 10% FBS and 2 mM L glutamine, supplemented with 100 units/mL penicillin, 100 μg/mL streptomycin, 500 μg/mL zeocin, and 200 μg/mL hygromycin and incubated at 37±0.5° C. with 5% CO₂.

Endogenous expression of integrin αvβ8 on the cell surface of LN 229 cells can bind and activate the latent TGF-β1 presented on the surface of TGFβ reporter cells by GARP. This activation allows TGF-β1 to bind to its cell surface receptor on the reporter cells, which signals through pSMAD and leads to the production of NanoLuc® luciferase. Constitutively expressed firefly luciferase was used for data normalization. The quantities of NanoLuc® luciferase and firefly luciferase were assessed using a Nano Glo® Dual Luciferase® reporter assay system (Promega; Madison, WI).

On the day of the assay, LN 229 cells were seeded into 96 well, flat, clear bottom, white polystyrene, tissue culture treated microplates (Corning; New York, NY) at a density of 40,000 cells/well by adding 42 μL of cells (952,000 cells/mL) diluted in test medium (high glucose DMEM supplemented with 10% heat inactivated FBS, 100 units/mL penicillin, 100 μg/mL streptomycin, and 2 mM glutamine). The anti-αvβ8 antibodies were diluted to a concentration of 100 μg/mL (333.3 nM) for antibody screening with single dose or serially diluted 4-fold for 11 dilution steps in test medium. Thereafter, 8 μL of diluted antibody were added to the LN 229 cells and incubated for 30 minutes (inside a biosafety cabinet for the first 10 minutes and then at 37° C. in an incubator with a 5% CO₂ atmosphere). Then, TGFβ reporter cells were seeded into the plate at a cell density of 20,000 cells/well by adding 30 μL of the cells (667,000 cells/mL). The plates of cells were incubated at 37° C. in an incubator with a 5% CO₂ atmosphere for 18-20 hours.

To measure the quantities of NanoLuc® and firefly luciferase, ONE Glo™ EX reagent (Promega) was pre warmed to room temperature, and 80 μL were added to the cells in each well. After incubation for 20 minutes at room temperature with strong agitation, firefly luciferase luminescence was measured using an EnSight® multimode plate reader (PerkinElmer; Waltham, MA). NanoDLR™ Stop & Glo® reagent (80 μL; Promega) was then added to each well. After incubation for 20 minutes at room temperature with strong agitation, the NanoLuc® luciferase luminescence was measured using an EnSight® multimode plate reader. The NanoLuc® luciferase signal was normalized to the firefly signal and multiplied by 1000. Half maximal inhibitory concentration (IC50) values were calculated from the titration curves using the Prism [Inhibitor] vs. Response—Variable Slope (Four Parameters) model (GraphPad Software; San Diego, CA). The percent inhibition was calculated using the following equation: % inhibition=100×[1−(X−MIN)/(MAX−MIN)], where MIN and MAX were the normalized NanoLuc® luciferase signals from reporter cells alone and co cultured with LN 229 cells in test medium, respectively.

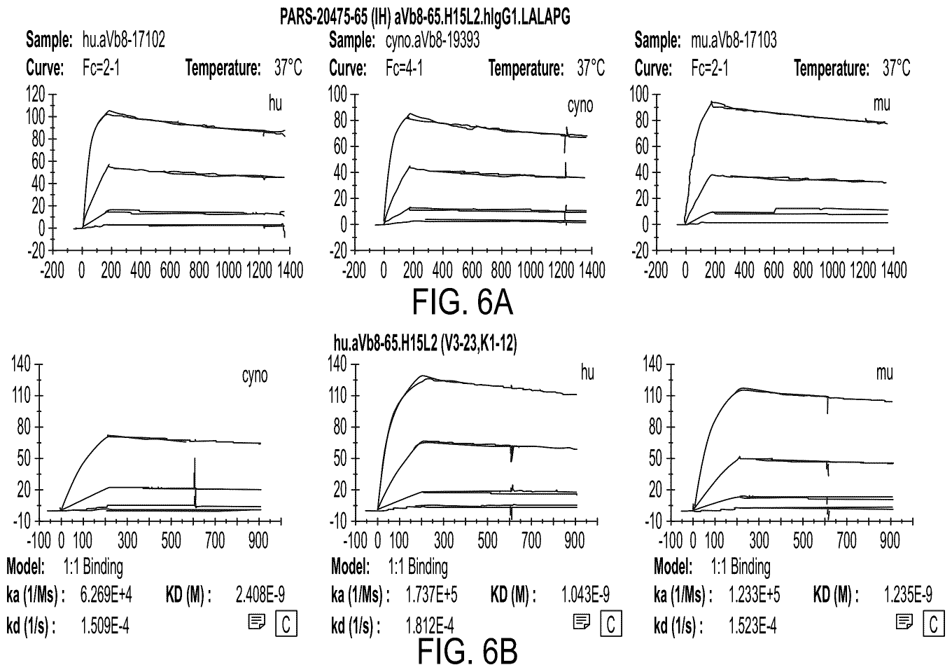

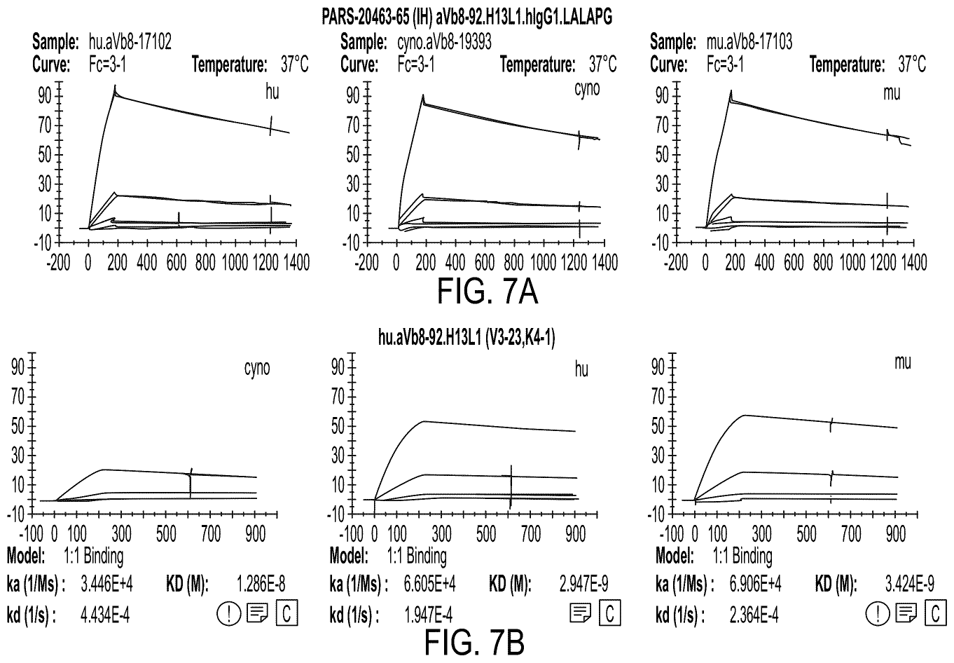

Humanized αVP8-65 (aVb8-65.H15L2.hIgG1.LALAPG) retained high binding affinity to human, cynomolgus and murine αVβ8. See Table 10 and FIGS. 6A-6B.

TABLE 10
Humanized αVβ8-65 Binding Assay Results
Experiment 1

| Ligand | Sample | ka (1/Ms) | kd (1/s) | KD (M) |
|---|---|---|---|---|
| PARS-20475-65 (IH) aVb8- | hu.aVb8-17102 | 5.40E+05 | 2.08E−04 | 3.84E−10 |
| 65.H15L2.hIgG1.LALAPG | cyno.aVb8-19393 | 3.41E+05 | 1.64E−04 | 4.80E−10 |
| | mu.aVb8-17103 | 1.71E+05 | 1.55E−04 | 9.06E−10 |

| | |
|---|---|
| hu.aVb1,3,5,6 | No binding |

Experiment 2

| Ligand | Sample | ka (1/Ms) | kd (1/s) | KD (nM) |
|---|---|---|---|---|
| hu.aVb8-65.H15L2 | hu.aVb8.17102 | 1.74E+05 | 1.81E−04 | 1.04 |
| | mu.aVb8.17103 | 1.23E+05 | 1.52E−04 | 1.23 |
| | cyno.aVb8.13650 | 6.27E+04 | 1.51E−04 | 2.41 |
| hC6D4 | hu.aVb8.17102 | 5.04E+04 | 2.45E−04 | 4.85 |
| | mu.aVb8.17103 | 6.64E+04 | 2.46E−04 | 3.71 |
| | cyno.aVb8.13650 | 1.03E+04 | 2.26E−04 | 21.9 |

Humanized αVβ38-92 (PARS-20463-65 (JH) aVb8-92.H13L1.hIgG1.LALAPG) retained high affinity to human, cynomolgus and murine αVβ8 as shown in Table 11 and FIGS. 7A-7B.

TABLE 11
Humanized αVβ8-92 Binding Assay Results
Experiment 1

| Ligand | Sample | ka (1/Ms) | kd (1/s) | KD (M) |
|---|---|---|---|---|
| PARS-20463-65 (IH) aVb8- | hu.aVb8-17102 | 2.80E+04 | 2.83E−04 | 1.01E−8 |
| 92.H13L1.hIgG1.LALAPG | cyno.aVb8-19393 | 2.55E+04 | 2.84E−04 | 1.11E−8 |
| | mu.aVb8-17103 | 3.14E+04 | 3.11E−04 | 9.91E−9 |

| | |
|---|---|
| hu.aVb1,3,5,6 | No binding |

Experiment 2

| Ligand | Sample | ka (1/Ms) | kd (1/s) | KD (nM) |
|---|---|---|---|---|
| hu.aVb8-92.H13L1 | hu.aVb8.17102 | 6.61E+04 | 1.95E−04 | 2.95 |
| | mu.aVb8.17103 | 6.91E+04 | 2.36E−04 | 3.42 |
| | cyno.aVb8.13650 | 3.45E+04 | 4.43E−04 | 12.9 |
| hC6D4 | hu.aVb8.17102 | 5.04E+04 | 2.45E−04 | 4.85 |
| | mu.aVb8.17103 | 6.64E+04 | 2.46E−04 | 3.71 |
| | cyno.aVb8.13650 | 1.03E+04 | 2.26E−04 | 21.9 |

Assessment of humanized αVP8-65 antibody binding to recombinant human, cynomolgus monkey, mouse and rat αVP8 proteins. Humanized αVP8-65 was generated as a 10.29 mg/mL solution in 20 mM histidine acetate (pH 5.5) and 150 mM NaCl. Recombinant human (PARS-17102), cynomolgus monkey (PARS-19393), mouse (PARS-17103), and rat (PARS-22180) αVβ8 proteins were prepared and stored at a temperature of −80° C.

The ability of the anti-αVP8-65 to bind to different αVβ8 proteins was assessed using SPR measurements (Karlsson et al. 1991) on a Biacore™ T200 (Cytiva Life Sciences; Marlborough, MA) instrument (Safsten et al. 2006). anti-αV08-65 was first captured on a Biacore™ Protein A biosensor chip (Cytiva Life Sciences) to achieve approximately 60 response units. Binding measurements were performed using a running buffer composed of 10 mM HEPES (pH 7.4), 150 mM NaCl, 0.5 mM CaCl₂), 0.5 mM MgCl₂, and 0.005% Surfactant P20 (Cytiva Life Sciences). A 3-fold dilution series of the analyte proteins (range, 0-100 nM in running buffer) was injected into the Biacore™ T200. All injections were performed over 180 seconds, with a dissociation time of 1200 seconds, at a flow rate of 100 μL/minute and a temperature of 37° C. Between sample injections, 10 mM glycine (pH 1.5) was injected to regenerate the sensor chip (twice, over 30 seconds at 10 μL/minute for each injection). To determine the binding kinetics and affinity constants of anti-αVP8-65 to the various species of the αVβ8 protein, the signal from the reference flow cell (FC1, with only protein A on the surface) was “double referenced” by subtracting the signal observed after injecting the sample over FC1, followed by subtracting the signal observed after injecting only running buffer. The kinetic constants were calculated using non-linear regression fitting of the data, according to a 1:1 Langmuir binding model, using Biacore™ Evaluation Software (Cytiva Life Sciences) for anti-αVP8-65 binding to recombinant human, cynomolgus monkey, mouse, and rat αVβ8 proteins.

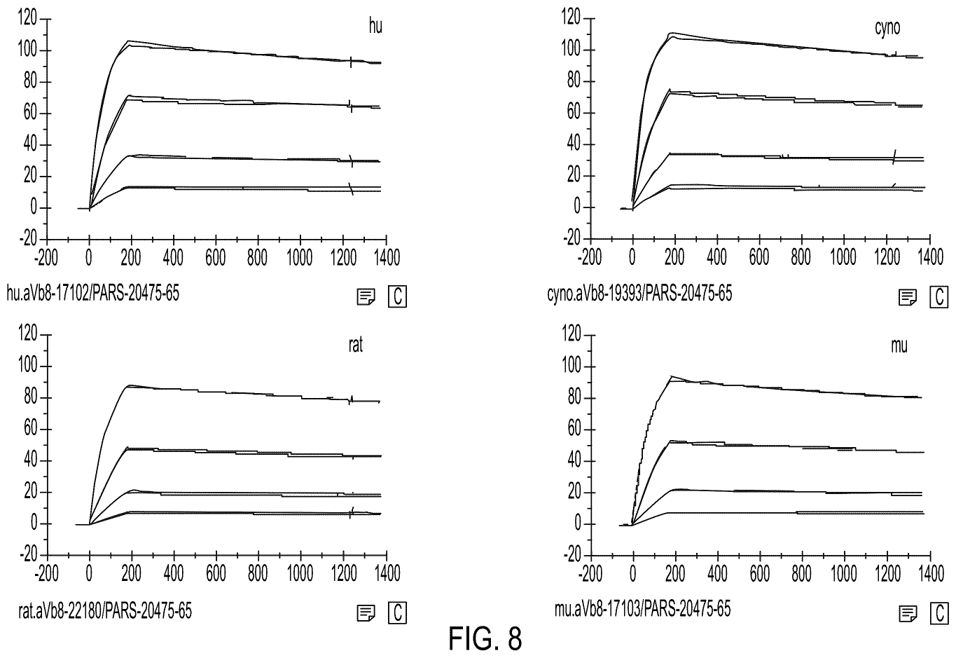

The anti-αVP8-65 antibody bound to recombinant human, cynomolgus monkey, mouse, and rat αVβ8 proteins with high affinities, and the average KD values determined using a 1:1 binding model were 0.50, 0.7, 0.94 and 1.0 nM, respectively, as shown in Table 12 and FIG. 8.

TABLE 12
Humanized αVβ8-65 Binding Assay Results

| Analyte | kₒₙ (1/Ms) | k_{off} (1/s) | K_{D} (nM) |
|---|---|---|---|
| hu.aVb8 | (1.75 ± 0.05) × 10⁵ | (8.8 ± 0.8) × 10⁻⁵ | 0.50 ± 0.03 |
| cyno.aVb8 | (1.8 ± 0.2) × 10⁵ | (1.3 ± 0.3) × 10⁻⁴ | 0.7 ± 0.1 |
| mu.aVb8 | (1.4 ± 0.4) × 10⁵ | (1.08 ± 0.06) × 10⁻⁴ | 0.94 ± 0.02 |
| rat.aVb8 | (1.04 ± 0.03) × 10⁵ | (1.1 ± 0.1) × 10⁻⁴ | 1.0 ± 0.1 |

cyno = cynomolgus monkey; hu = human; K_{D} = equilibrium dissociation constant; k_{off} = dissociation rate constant; kₒₙ = association rate constant; mu = mouse.

### Example 3: Affinity of Anti-αVP8 Monoclonal Antibodies for Human αVP8

The abilities of humanized αvβ8-65, hC6D4, and hADWA11-2.4 to bind to human aVb8 integrin protein were assessed using SPR measurements (Karlsson et al. 1991) on a Biacore™ T200 (Cytiva Life Sciences; Marlborough, MA) instrument (Safsten et al. 2006). All antibodies tested in the assays were generated in house. Before use in the study, the materials were stored in a refrigerator set to maintain a temperature range of 2-8° C.

In SPR based biosensors, the refractive index angle changes near a surface are monitored and translated to measured response signals. If a protein target (“ligand”) is covalently immobilized on the sensor chip surface, SPR can be used to monitor the non-covalent interaction of a binding partner (“analyte”) injected over the surface. These “real time” measurements of analyte binding can be used to determine both the kinetics and affinity of the interaction.

The antibodies were first captured either on a Biacore™ Protein A biosensor chip (Cytiva Life Sciences) or on an anti-murine Fc chip to achieve approximately 60 response units. Binding measurements were performed using a running buffer composed of 10 mM HEPES (pH 7.4), 150 mM NaCl, 0.5 mM CaCl₂), and 0.005% Surfactant P20 (Cytiva Life Sciences). For comparison between αvβ8-65 and hC6D4, a 5-fold dilution series of human αvβ8 (range, 0-100 nM in running buffer) was injected into the Biacore™ T200. For comparison between αvβ8-65 and hADWA11-2.4, a 5-fold dilution series of human αvβ8 (range, 0-100 nM in running buffer) was injected. For comparison of all three antibodies in the murine Fc format, a 5-fold dilution series of human αvβ8 (range, 0-50 nM in running buffer) was injected. All injections were performed over 180 seconds, with a dissociation time of 1200 seconds, at a flow rate of 100 μL/minute and a temperature of 37° C. Between sample injections, a regeneration reagent (10 mM glycine pH 1.5 for protein A chip; 10 mM glycine pH 1.7 for anti-murine Fc chip) was injected to regenerate the sensor chip (twice, over 30 seconds at 10 μL/minute for each injection). To determine the binding kinetics and affinity constants of the antibodies to human αvβ8 protein, the signal from the reference flow cell (FC1, with only protein A on the surface) was “double referenced” by subtracting the signal observed after injecting the sample over FC1, followed by subtracting the signal observed after injecting only running buffer. The kinetic constants were calculated using nonlinear regression fitting of the data, according to a 1:1 Langmuir binding model, using Biacore™ Evaluation Software (Cytiva Life Sciences).

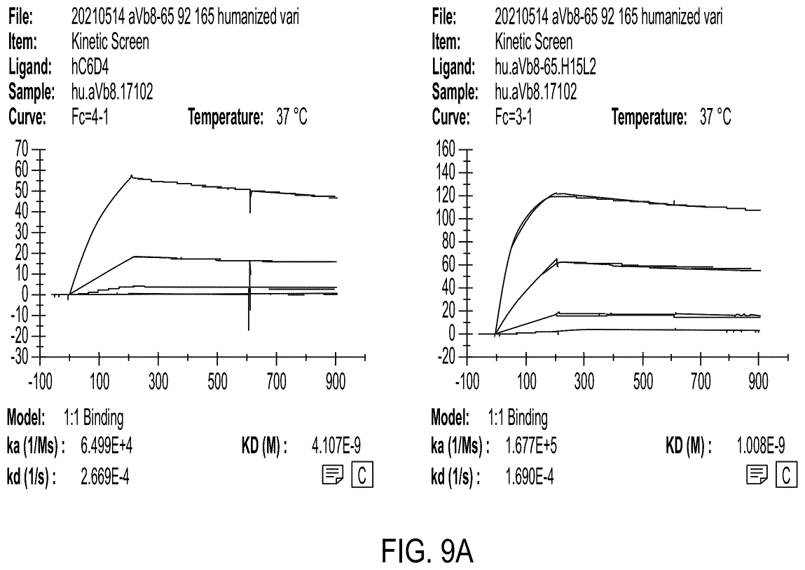

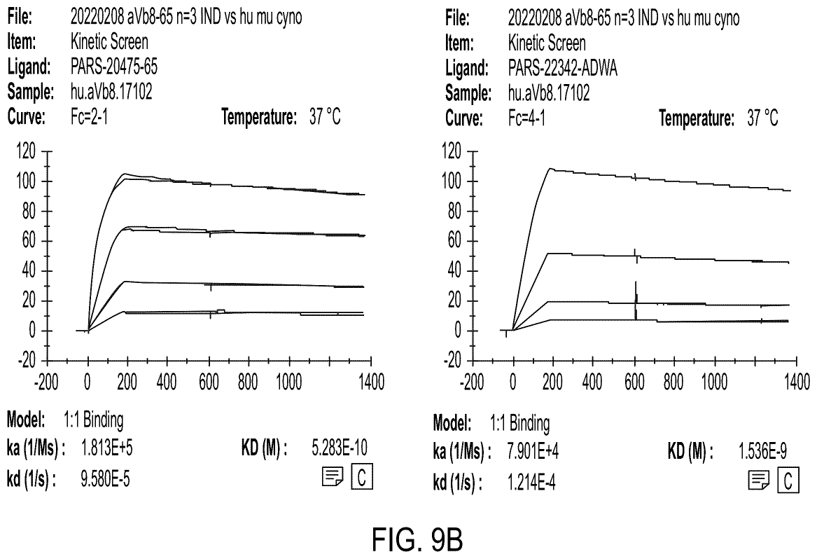

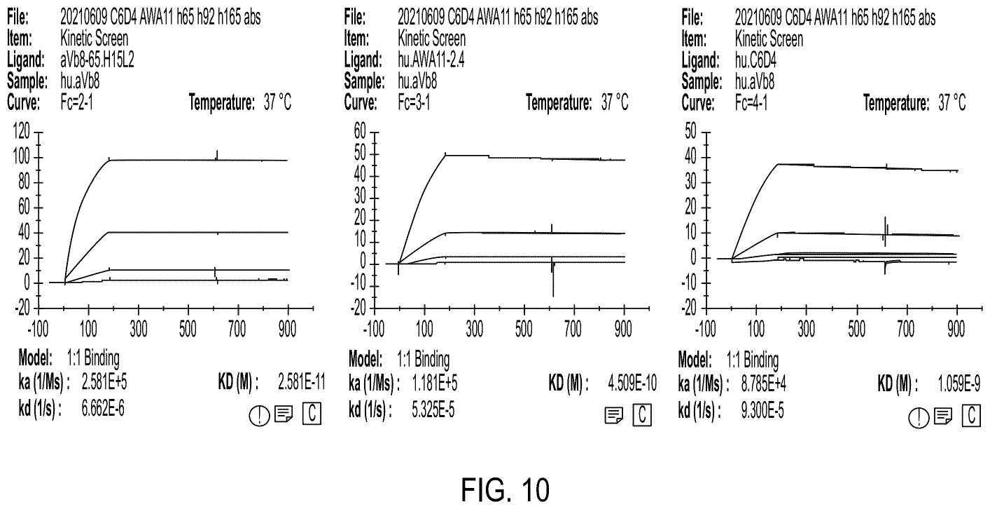

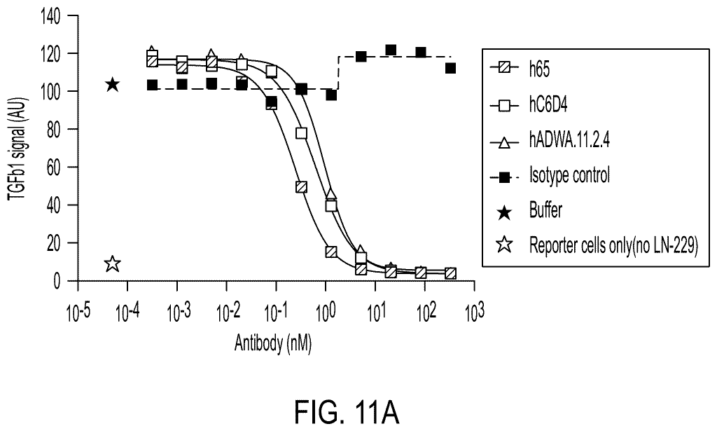

The values of the kinetic constants kon, koff, and KD for the binding of αvβ8-65, hC6D4 and ADWA11-2.4 to the various species of αvβ8 proteins are summarized in Table 13. A 1:1 Langmuir binding model was used to determine the KD values. The sensograms observed for the bindings of the various αvβ8 proteins to the captured antibody are shown in FIG. 9A, FIG. 9B, and FIG. 10. The solid lines overlaid on the experimental curves in the figure represent the results of analyses performed using the 1:1 binding model, indicating that the 1:1 binding model is adequate to describe this interaction.

TABLE 13
Kinetic and Affinity Constants for the Binding of
Recombinant human αvβ8 Protein to Captured
αvβ8-65, hC6D4, hADWA11-2.4 as Determined
Using Surface Plasmon Resonance at 37° C.

| Clone | kₒₙ (1/Ms) | k_{off} (1/s) | K_{D} (nM) |
|---|---|---|---|
| αvβ8-65 | 1.677 × 10⁵ | 1.690 × 10⁻⁴ | 1.008 |
| hC6D4 | 6.499 × 10⁴ | 2.669 × 10⁻⁴ | 4.107 |
| αvβ8-65 | 1.813 × 10⁵ | 9.580 × 10⁻⁵ | 0.528 |
| hADWA11-2.4 | 7.901 × 10⁴ | 1.214 × 10⁻⁴ | 1.536 |
| αvβ8-65 | 2.581 × 10⁵ | <1 × 10⁻⁵ * | <0.0258 |
| hC6D4 | 8.785 × 10⁴ | 9.300 × 10⁻⁵ | 1.059 |
| hADWA11-2.4 | 1.181 × 10⁵ | 5.325 × 10⁻⁵ | 0.4509 |

hu = human; K_{D} = equilibrium dissociation constant; k_{off} = dissociation rate constant; kₒₙ = association rate constant; mu = mouse.
* The kd for aVb8-65 is below the T200 instrument detection limit for dissociation rate constant (kd) of 10⁻⁵ s⁻¹

### Example 4: Potency of Anti-αVP8 Monoclonal Antibodies for Human αVP8

All antibodies have mIgG2a.LALAPG isotype. All antibodies tested in the assays were stored in a refrigerator set to maintain a temperature range of 2-8° C.

LN-229 cells that are derived from a human glioblastoma and express αvβ8 endogenously were obtained from the Genentech cell bank (gCell). The cells were maintained in culture medium (high glucose Dulbecco's Modified Eagle Medium (DMEM) containing 10% fetal bovine serum, 2 mM L glutamine, 100 units/mL penicillin, and 100 μg/mL streptomycin) at 37±0.5° C. with a 5% CO₂ atmosphere.

3T3-Nano cells, a TGFβ reporter cell line was generated by stable transfection of 3T3 cells obtained from gCell. The cells were derived from a mouse fibroblast cell line, with a SMAD inducible NanoLuc® luciferase reporter gene and a constitutively expressed firefly luciferase gene. The cells were maintained in high glucose DMEM with 10% FBS and 2 mM L glutamine, supplemented with 100 units/mL penicillin, 100 μg/mL streptomycin, and 500 μg/mL zeocin and were incubated at 37±0.5° C. with 5% CO₂.

3T3-Nano-human GARP/LTGF-β1 (WT) reporter cells used to determine potency of the antibodies blocking the integrin αvβ8 mediated activation of TGF-β1 (WT) were generated by stable transfection of 3T3-Nano cells with human GARP and latent TGF-β1 (WT).

3T3-Nano-human GARP/LTGF-31 (non-releasable, NR) reporter cells used to determine potency of the antibodies blocking the integrin avb8 mediated activation of LTGF-β1(NR) were generated by transient transfection of 3T3-Nano cells with human GARP and latent TGF-β1(NR).

3T3-Nano-human GARP/LTGF-β3 (WT) reporter cells used to determine potency of the antibodies blocking the integrin avb8 mediated activation of latent TGF-β3 (WT) were generated by transient transfection of 3T3-Nano cells with human GARP and latent TGF-β3 (WT).

Co cultures of LN-229 cells endogenously expressing integrin αvβ8 as well as TGF β reporter cells, 3T3-Nano-human GARP/LTGF-β3 (WT), 3T3-Nano-human GARP/LTGF-β1 (NR) or 3T3-Nano-human GARP/LTGF-β3 (WT) were used to evaluate the blocking activity of antibodies against integrin avb8 mediated activation of LTGF-β1 (WT), LTGF-β1 (NR) or LTGF β3 (WT)β respectively. Expression of integrin αvβ8 on the cell surface of LN-229 cells can bind and activate the latent TGF β1(WT or NR) and latent TGFβ presented on the surface of TGFβ reporter cells by GARP. This activation allows TGF β1 (WT or NR) or LTGF 33 (WT) to bind to its cell surface receptor on the reporter cells, which signals through pSMAD and leads to the production of NanoLuc® luciferase. Constitutively expressed firefly luciferase was used for data normalization. The quantities of NanoLuc® luciferase and firefly luciferase were assessed using a Nano Glo® Dual Luciferase® reporter assay system (Promega; Madison, WI).

On the assay day 1, 6×10⁶ 3T3-Nano cells in 12 mL of culture medium (high glucose DMEM with 10% FBS, 2 mM L glutamine and 500 μg/mL zeocin) were seeded into a T75 cell culture flask (Corning; New York, NY) and incubated at 37° C. in an incubator with a 5% CO₂ atmosphere for 20-24 hours.

On the assay day 2, 3T3-Nano cells were washed with 12 ml transfection medium (high glucose DMEM with 10% FBS and 2 mM L glutamine) and then transfected with expression vectors of human GARP and latent TGF β1(NR) (3T3-Nano-human GARP/LTGF β 1(NR)) or with human GARP and latent TGF β 3 (WT) (3T3-Nano-human GARP/LTGF β 3 (WT)) by Lipofectamine 3000 (Thermo Fisher Scientific; Waltham, MA) per manufacture's instruction. The flasks were incubated at 37° C. in an incubator with a 5% CO₂ atmosphere for 22-24 hours.

On the assay day 3, LN-229 cells were seeded into 96 well, flat, clear bottom, white polystyrene, tissue culture treated microplates (Corning; New York, NY) at a density of 40,000 cells/well by adding 42 μL of cells (952,000 cells/mL) diluted in test medium (high glucose DMEM supplemented with 10% heat inactivated FBS, 100 units/mL penicillin, 100 μg/mL streptomycin, and 2 mM glutamine). The test material was diluted to a concentration of 500 μg/mL (3333.3 nM) and serially diluted 4-fold for 11 dilution points in phosphate-buffered saline (PBS). Thereafter, 8 μL of diluted antibody or PBS were added to the LN-229 cells and incubated for 30 minutes (inside a biosafety cabinet for the first 10 minutes and then at 37° C. in an incubator with a 5% CO₂ atmosphere). Then, the reporter cells, 3T3-Nano-human GARP/LTGF β1 (WT), 3T3-Nano-human GARP/LTGF β1(NR) or 3T3-Nano-human GARP/LTGF 33 (WT) cells were seeded into the assay plate at a cell density of 20,000 cells/well by adding 30 μL of the cells (667,000 cells/mL). The plates of cells were incubated at 37° C. in an incubator with a 5% CO₂ atmosphere for 18-20 hours.

On the assay day 4, the levels of latent TGF-β1(WT or NR) or latent TGF 33 (WT) activation and TGF-β reporter cell normalization were determined by measuring the quantities of NanoLuc® and firefly luciferase, respectively. ONE Glo™ EX reagent (Promega) was pre warmed to room temperature, and 80 μL were added to the cells in each well. After incubation for 20 minutes at room temperature with strong agitation, firefly luciferase luminescence was measured using an EnSight® multimode plate reader (PerkinElmer; Waltham, MA). NanoDLR™ Stop & Glo® reagent (80 μL; Promega) was then added to each well. After incubation for 20 minutes at room temperature with strong agitation, the NanoLuc® luciferase luminescence was measured using an EnSight® multimode plate reader. The NanoLuc® luciferase signal was normalized to the firefly signal and multiplied by 1000. Half maximal inhibitory concentration (IC50) values were calculated from the titration curves using the Prism [Inhibitor] vs. Response—Variable Slope (Four Parameters) model (GraphPad Software; San Diego, CA).

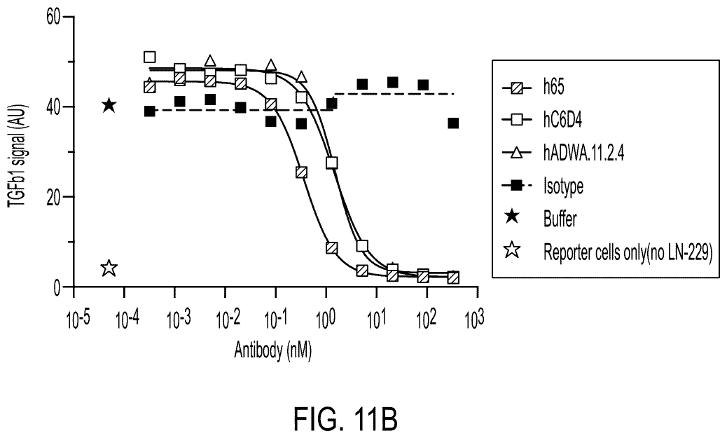

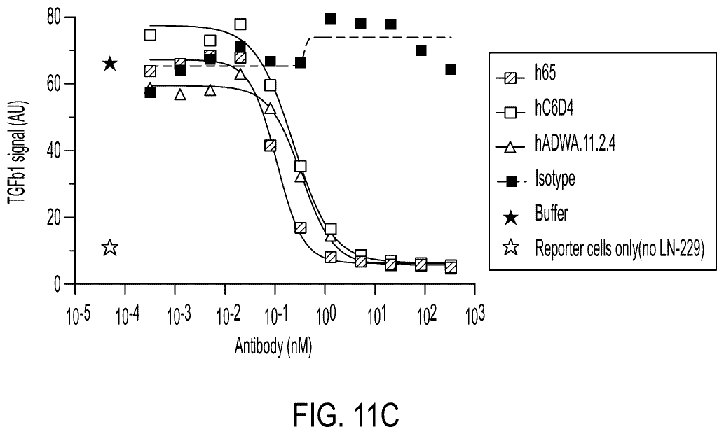

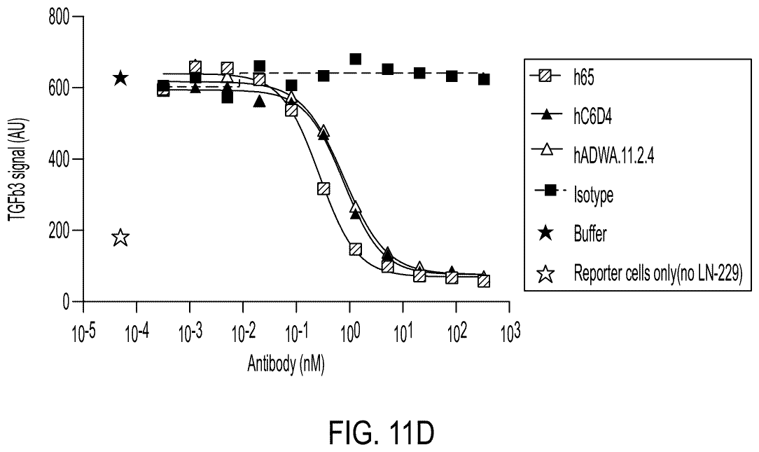

The in vitro cell based potency assay in a co-culture between LN-229 and 3T3-Nano-human GARP/LTGFβ1 (WT) is shown in FIGS. 11A (Experiment #1) and 11B (Experiment #2). The in vitro cell based potency assay in a co-culture between LN-229 and 3T3-Nano-human GARP/LTGFβ1 (non-releasable (NR)) is shown in FIG. 11C (Experiment #3). The in vitro cell based potency assay in a co-culture between LN-229 and 3T3-Nano-human GARP/LTGFβ33 (WT) is shown in FIG. 11D (Experiment #4). A summary of the IC50 (nM) is depicted in Table 14.

TABLE 14
Summary of IC50 (nM)

| Experiment # | Antibody | IC50 (nM) |
|---|---|---|
| #1 | clone 65 | 0.25 |
| | ADWA11 | 0.95 |
| | C6D4 | 0.63 |
| | Isotype control | >333.3 |
| #2 | clone 65 | 0.36 |
| | ADWA11 | 1.51 |
| | C6D4 | 1.45 |
| | Isotype control | >333.3 |
| #3 | clone 65 | 0.11 |
| | ADWA11 | 0.34 |
| | C6D4 | 0.24 |
| | Isotype control | >333.3 |
| #4 | clone 65 | 0.27 |
| | ADWA11 | 0.79 |
| | C6D4 | 0.78 |
| | Isotype control | >333.3 |

### Example 5: Competition Assay with anti-αVβ8 Monoclonal Antibodies

All antibodies tested were maintained at a temperature range of 2-8° C.

The EMT6 murine mammary carcinoma and HCC 1159 human ovary carcinoma cell lines were obtained from the American Type Culture Collection (ATCC, Manassas, VA). Cells were cultured in RPMI (Gibco, USA) supplemented with 10% of FBS (Gibco, USA) under standard conditions (37° C. in humidified atmosphere containing 5% C02.

Cells were detached with 2.5 mM EDTA from the culture and incubated with Mouse BD FC Block™ (5 μg/ml; Catalog No. 553142, BD Biosciences, San Jose, CA) for 30 minutes on ice. Then cells were stained in 96 wells with different antibody combinations (PE-Clone65 with AF647-hC6D4 or PE-Clone65 with AF647-hADWA11) at different concentrations (0 ug/ml to 40 ug/ml) on ice for 1 hour. Last, cells were incubated with LIVE/DEAD® Aqua Fixable Dead Cell (Catalog No. L34957; Thermo Fisher Scientific; Waltham, MA) for 30 minutes on ice and then fixed with 1% PFA. Flow Cytometry data were collected with a BD FACS symphony (BD Biosciences). Flow data were analyzed in FlowJo (version 10.8.1). Half maximal inhibitory concentration (IC50) values were calculated from the titration curves using the Prism [Inhibitor] vs. Response—Variable Slope (Four Parameters) model (GraphPad Software; San Diego, CA).

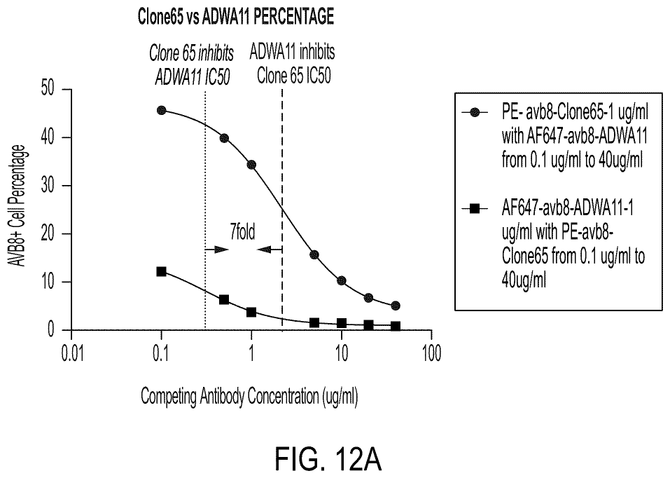

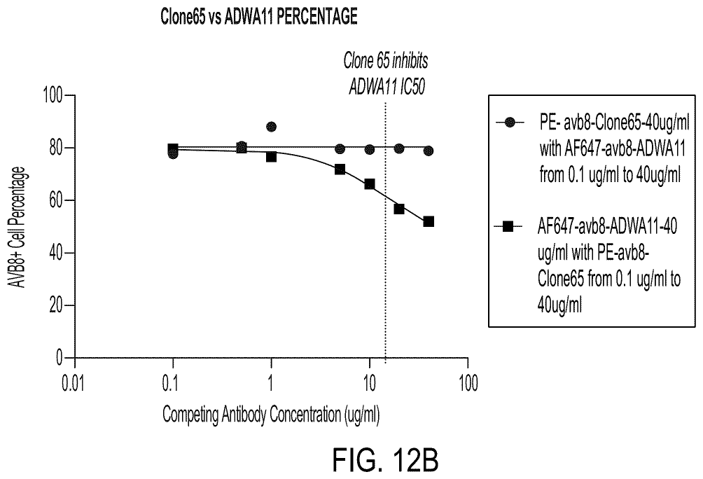

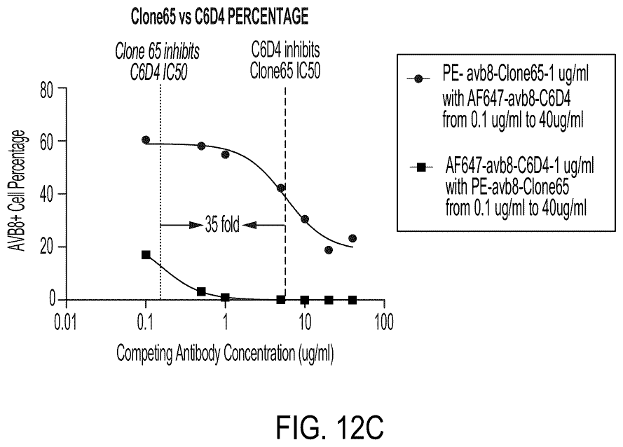

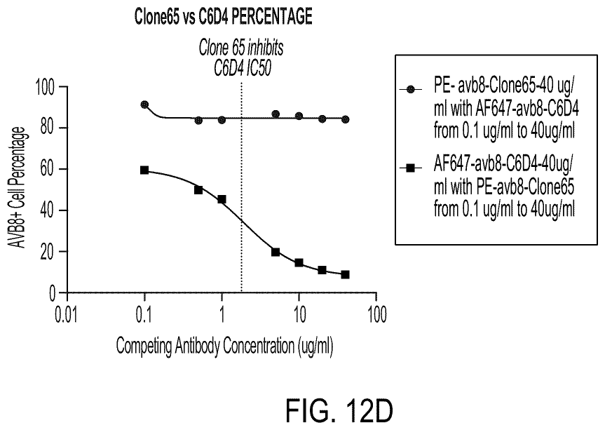

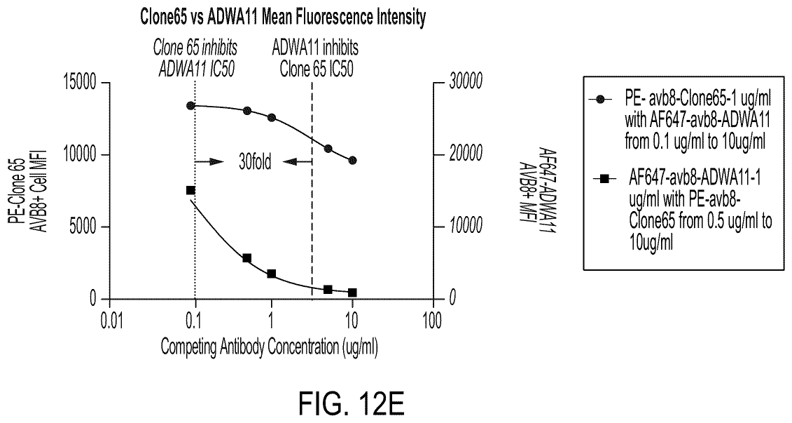

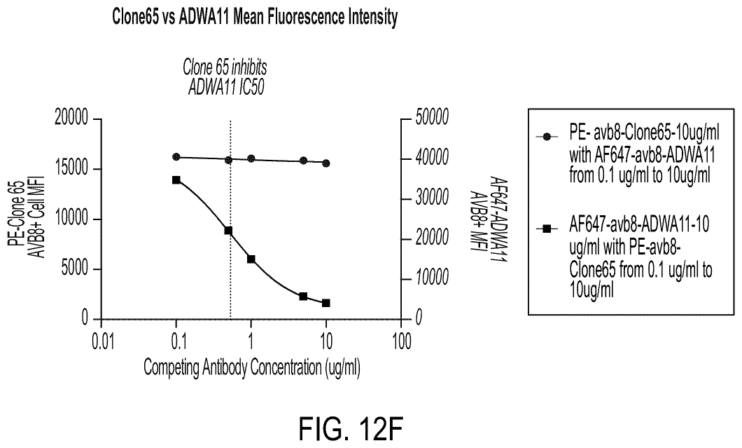

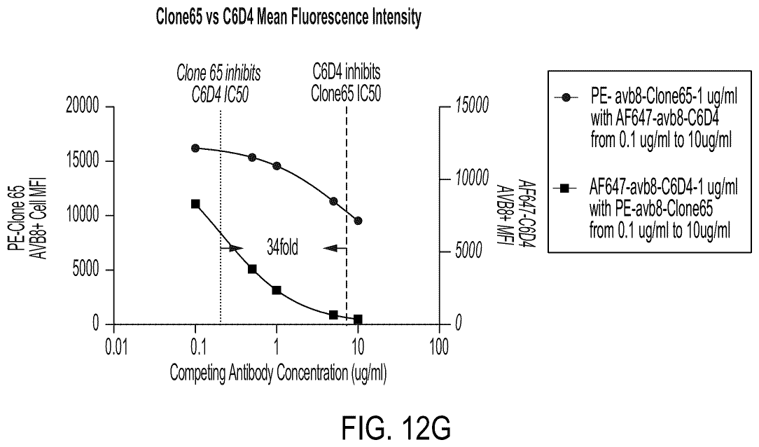

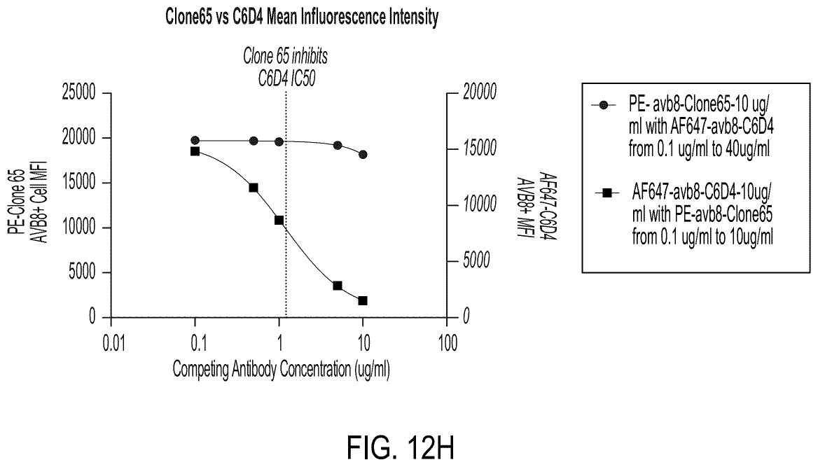

The competition assay between anti-αVP8-65 and ADWA11 in the EMT6 cell line shown in FIGS. 12A (1 μg/mL) and 12B (40 μg/mL). The competition assay between anti-αVP8-65 and C6D4 in the EMT6 cell line is shown in FIGS. 12C (1 μg/mL) and 12D (40 μg/mL). The competition assay between anti-αVP8-65 and ADWA11 in the HCC1159 cell line is shown in FIGS. 12E (1 μg/mL) and 12F (10 μg/mL). The competition assay between anti-αVP8-65 and C6D4 in the HCC1159 cell line is shown in FIGS. 12G (1 μg/mL) and 12H (10 μg/mL). A summary of IC50 (mg/mL) is shown in Table 15.

TABLE 15
Summary of IC50 (mg/mL)

| Figure | Antibody | IC50 (mg/ml) |
|---|---|---|
| FIG. 12A | Clone 65 | 0.3072 |
| | ADWA11 | 2.918 |
| FIG. 12B | Clone 65 | 15.08 |
| FIG. 12C | Clone 65 | 0.1603 |
| | C6D4 | 5.740 |
| FIG. 12D | Clone 65 | 1.926 |
| FIG. 12E | Clone 65 | 0.1063 |
| | ADWA11 | 3.161 |
| FIG. 12F | Clone 65 | 0.5242 |
| FIG. 12G | Clone 65 | 0.2086 |
| | C6D4 | 7.215 |
| FIG. 12H | Clone 65 | 1.182 |

### Example 6: Pharmacokinetic (PK) Profile of Humanized αVP8 Antibodies in Various Animal Models

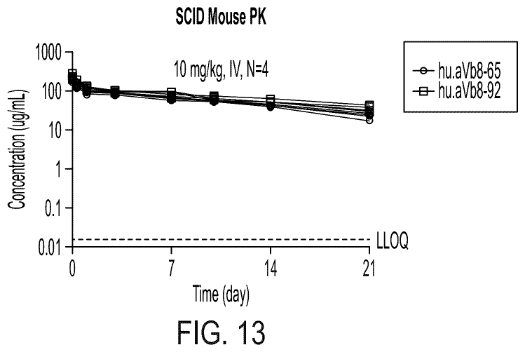

SCID Mice. SCID mice were IV administered 10 mg/kg of hu.αVP8-65 or hu.αVP8-92 and serum concentration of both antibodies was measured over 21 days as shown in FIG. 13. The PK profile of αVP8-65 or hu.αVP8-92 is provided in Table 16.

TABLE 16
Pharmacokinetics Profile of Humanized αvβ8 antibodies in SCID mice.

| | ⁺Cₘₐₓ | AUCₐₗₗ | AUC_{inf} | CL | Vₛₛ | |
|---|---|---|---|---|---|---|
| Group | (ug/mL) | (ug*day/mL) | (ug*day/mL) | (mL/day/kg) | (mL/kg) | AUC_{%extrap} |
| hu.aVb8-65 | 208.6 ± | 1197.2 ± | 1485 ± | 6.78 ± | 86.1 ± | 19 ± |
| | 23.3 | 65.3 | 142.7 | 0.708 | 8.32 | 5.9 |
| hu.aVb8-92 | 248.3 ± | 1491 ± | 2211 ± | 4.58 ± | 84.15 ± | 32.1 ± |
| | 26.5 | 102.8 | 287.9 | 0.626 | 4.58 | 5.3 |

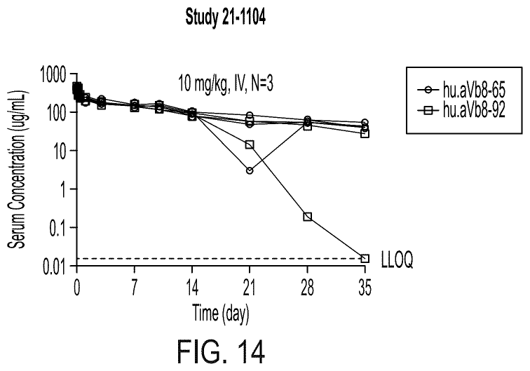

Cynomolgus monkeys. Cynomolgus monkeys were IV administered 10 mg/kg of hu.aVb8-92 or hu.aVb8-65 and serum concentration of both antibodies were measured over 35 days as shown in FIG. 14. The observed Cmax was higher (˜1.5 fold) than anticipated based on cynomolgus serum volume (Table 17). Lower than typical Vss (˜80 ml/kg) for cynomolgus was also observed (Table 17).

TABLE 17
Pharmacokinetics Profile of Humanized αvβ8 antibodies in Cynomolgus monkeys.

| | ⁺Cₘₐₓ | AUCₐₗₗ | AUC_{inf} | AUC₀₋₁₄ | CL | Vₛₛ | |
|---|---|---|---|---|---|---|---|
| Group | (ug/mL) | (ug*day/mL) | (ug*day/mL) | (ug*day/mL) | (mL/day/kg) | (mL/kg) | AUC_{%Extrap.} |
| hu.aVb8-65 | 387 ± | 3659 ± | 4804 ± | 2268 ± | 2.14 ± | 49.1 ± | 23.01 ± |
| | 30.43 | 451 | 956 | 222 | 0.42 | 3.9 | 6.8 |
| hu.aVb8-92 | 419 ± | 3190 ± | 3655 ± | 2140 ± | 2.74 ± | 46 ± | 12.7 ± |
| | 62.6 | 151 | 130 | 153 | 0.98 | 3 | 1 |

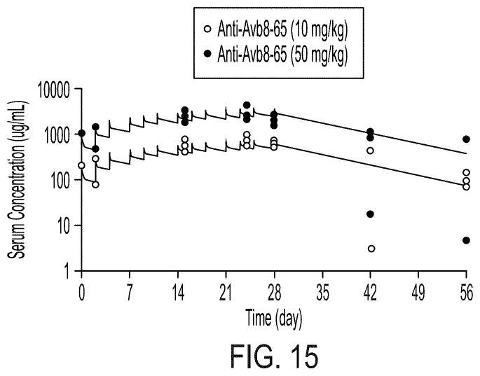

CD-1 Mice. Safety of hu.aVb8-65 antibody was measured in a 56 day study at two separate doses (10 mg/kg or 50 mg/kg) in CD-1 mice (FIG. 15). The study design is shown in Table 18. Mice were IV administered the two separate doses three times per week for four weeks. Only one animal was found dead on day 30 (50 mg/kg of hu.aVb8-65). All other animals survived to scheduled necropsy. There were no clinical observations, effects on body weight or toxicological significance in CD-1 mice administered either dose.

TABLE 18
Pharmacokinetics Profile of Humanized αVβ8 antibodies in CD-1 mice.

| | Dose^{a} | Cumulative Dose | Number of Animals (M/F) | | |
|---|---|---|---|---|---|
| Treatment | (mg/kg TIW) | (mg/kg/week) | Terminal (D29) | Recovery (D57) | TK |
| Vehicle | 0 | 0 | 5/5 | 3/0 | N/A |
| hu.aVb8-65 | 10 | 30 | 5/5 | N/A | 6/0 |
| hu.aVb8-65 | 50 | 150 | 5/5 | 3/0 | 6/0 |

### Example 7: In Vivo Anti-Tumor Efficacy of Humanized αvβ8 Antibodies

All anti-αVβ8 antibodies were on a mouse IgG2a.LALAPG isotype. Murine (Mu) IgG1 anti-PD-L1 6E11 monoclonal antibody (Mu anti-PD-L1) was generated by immunization of PD-L1 knockout mice with PF-L1-Fc fusion protein and cloned onto a murine IgG1 isotype antibody. Mu IgG1 anti-glycoprotein 120 (Mu IgG1 anti-gp120) is the control for Mu anti-PD-L1. Mu IgG2a LALAPG anti-gp120 (Mu IgG2a anti-gp120) is the control for the anti-αVβ8 antibodies. All antibodies tested in the assays were generated in house. Before use in the study, the materials were stored in a refrigerator set to maintain a temperature range of 2-8° C.

The EMT6 murine mammary carcinoma cell line, obtained from American Type Culture Collection (Manassas, VA), was cultured in RPMI 1640 medium containing 1% L-glutamine and 10% fetal bovine serum (FBS; Catalog No. F2442; Sigma-Aldrich, St Louis, MO). The cells were detached using 0.5% trypsin in an EDTA-containing phosphate-buffered saline and collected in RPMI 1640 containing 1% L-glutamine and 10% FBS. The collected cells were centrifuged, washed once with Hanks' Balanced Salt Solution (HBSS), counted, and resuspended in a 1:1 solution of HBSS and Matrigel (Corning; Bedford, MA) at a density of 1×10⁶ cells/mL before inoculation into animals.

Female Balb/c mice (8-9 weeks old, ˜20 g at study initiation) were obtained from Charles River Laboratories (Hollister, CA). The mice were housed in standard rodent micro-isolator cages and were acclimated to study conditions for at least 3 days before tumor cell implantation. Only animals that appeared to be healthy and free of obvious abnormalities were used for the study.

Mice were inoculated in the left mammary fat pad #5 with 1×10⁵ syngeneic EMT6 cells suspended in 100 μL of HBSS:Matrigel. Tumors were monitored until they reached a volume of ˜180 mm3 (7 days after inoculation). On Day 0 of the study, the mice were randomly assigned to 6 groups (9 mice/group), based on tumor volume. Antibodies were administered twice per week for 3 weeks (intravenously for the first dose and intraperitoneally, thereafter). Mice were treated with Isotype control, aPD-L1 or a combination of aPDL1 and one of the anti-αvβ8 antibodies. All antibodies were administrated at 10 mg/kg. All dosing concentrations were calculated based on a mean body weight of 18.5 g for the Balb/c mouse strain used in this study. Each antibody stock solution was diluted using a histidine buffer (20 mM histidine acetate, 240 mM sucrose, 0.02% polysorbate-20, pH 5.5). Each test material was diluted to a concentration that allowed the administration of a dose of 10 mg/kg. Tumor measurements, body weights, and general clinical observations were recorded twice per week over the course of the study.

Tumors were measured using calipers, and tumor volumes were calculated using the modified ellipsoid formula. Mice were euthanized if their tumor volumes exceeded 1500 mm3, their tumors ulcerated, or their body weight loss was equal or more than 20% of their starting weight. All animal studies were approved by the Genentech Institutional Animal Care and Use Committee. The lengths and widths of the tumors were measured using UltraCal-IV calipers (Model 54-10-111; Fred V. Fowler Co; Newton, MA). The following formula was used, in Excel (Version 11.5.6; Microsoft; Redmond, WA), to calculate tumor volume: Tumor volume (mm3)=(length×width²)×0.5.

Analyses and comparisons of tumor growth were performed using a package of customized functions in R (Version 4.1.0; R Foundation for Statistical Computing; Vienna, Austria) that integrates software from open-source packages (e.g., lme4, mgcv, gamm4, multcomp, settings, and plyr) and several packages from tidyverse (e.g., magrittr, dplyr, tidyr, and ggplot2) (Forrest et al., Generalized additive mixed modeling of longitudinal tumor growth reduces bias and improves decision making in translational oncology, Cancer Res 2020; 80(22):5089-97). Briefly, as tumors generally exhibit exponential growth, tumor volumes were subjected to natural log transformation before analysis. All raw tumor volume measurements less than 8 mm3 were judged to reflect complete tumor absence and were converted to 8 mm3 prior to natural log transformation. Additionally, all raw tumor volume measurements less than 16 mm3 were considered miniscule tumors too small to be measured accurately and were converted to 16 mm3 prior to natural log transformation. A generalized additive mixed model (GAMM) was then applied to fit the temporal profile of the log-transformed tumor volumes in all study groups with regression splines and automatically generated spline bases. This approach addresses both repeated measurements from the same study subjects and moderate dropouts before the end of the study.

Strong anti-tumor responses that resulted in reductions in tumor size were tracked as partial responses (PRs, defined as animals in the group that had their raw tumor volume measurement drop by more than 50% at any point during their participation in the study, but whose final raw tumor volume measurement was still greater than 8 mm3) and complete responses (CRs, defined as animals in the group whose final raw untransformed tumor volume measurement was less than 8 mm3. For an animal to be counted as a CR, its final tumor volume measurement cannot indicate the existence of a tumor).

Several studies were conducted following the described procedure. For each study the % of CR above anti-PD-L1 was calculated as follows: % CR=n CR/n mice in the group of treatment; % CR above anti-PD-L1=% CR of combination group −% CR of anti-PD-L1 group.

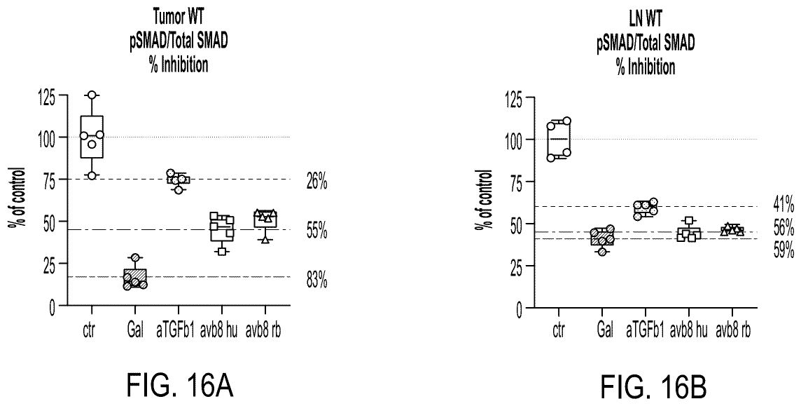

rb.avb8-65 and hu.avb8-65 show similar inhibition of pSMAD2/3 in tumor and lymph node (FIGS. 16A and 16B). EMT6 Thy1.1 WT tumor bearing mice were treated with aPD-L1 and GP120 (ctr) or mice were treated with aPD-L1 and Galunisertib (1 hr prior to takedown; SMI of TGFbR2), anti-TGFb1 21D1, anti-hu.avb8.65.m2a.LALAPG, or anti-rb.avb8.65.m2a.LALAPG. Around 55-56% of pSMAD2/3 was inhibited with rb.avb8-65 and hu.avb8-65 administration.

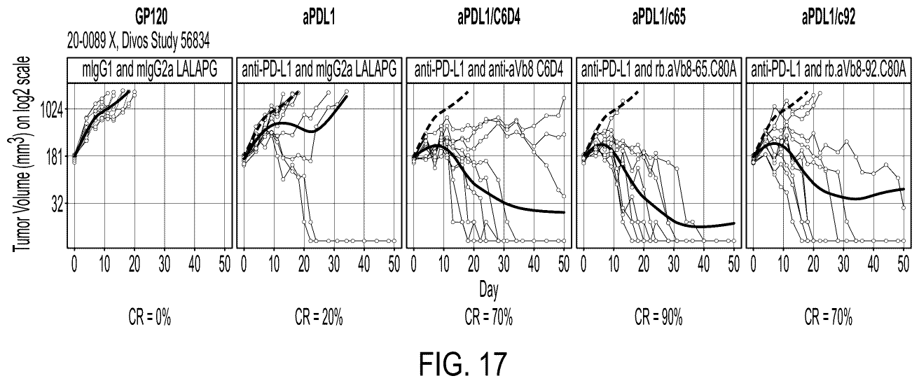

Next, anti-tumor efficacy of the humanized and rabbit antibodies were studied by co-injecting mice with the antibodies and aPD-L1. Tumor volume was measured across days. The humanized and rabbit αVP8 antibodies were superior to Mu.C6D4 (FIG. 17).

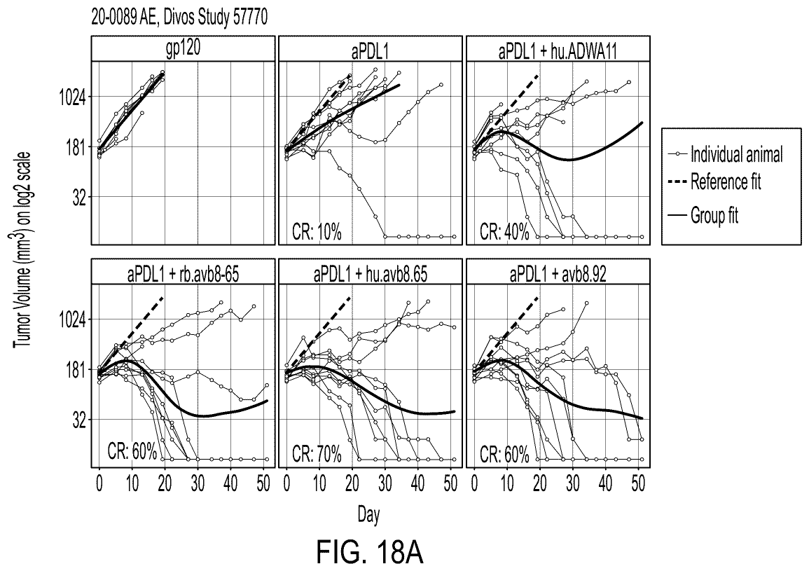

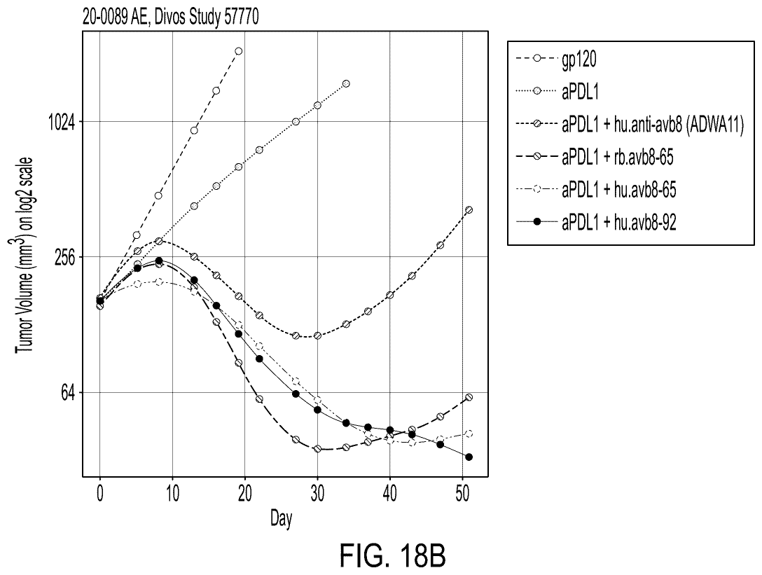

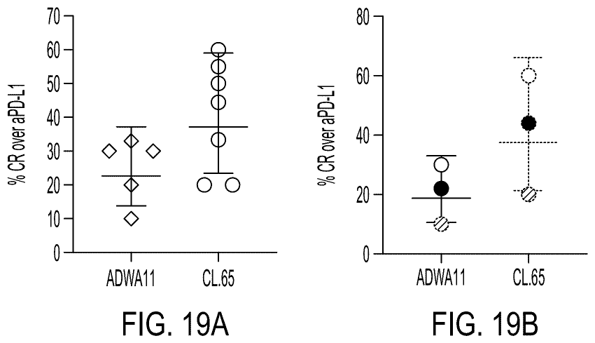

Hu.avb8-65 and -92 also showed better complete response (CR) than benchmark (ADWA11) in EMT6 tumor model in combination with anti-PD-L1 (FIGS. 18A and 18B). Hu.avb8-65 showed better CR than ADWA11 in the EMT6 tumor model in combination with anti-PD-L1 (FIGS. 19A and 19B).

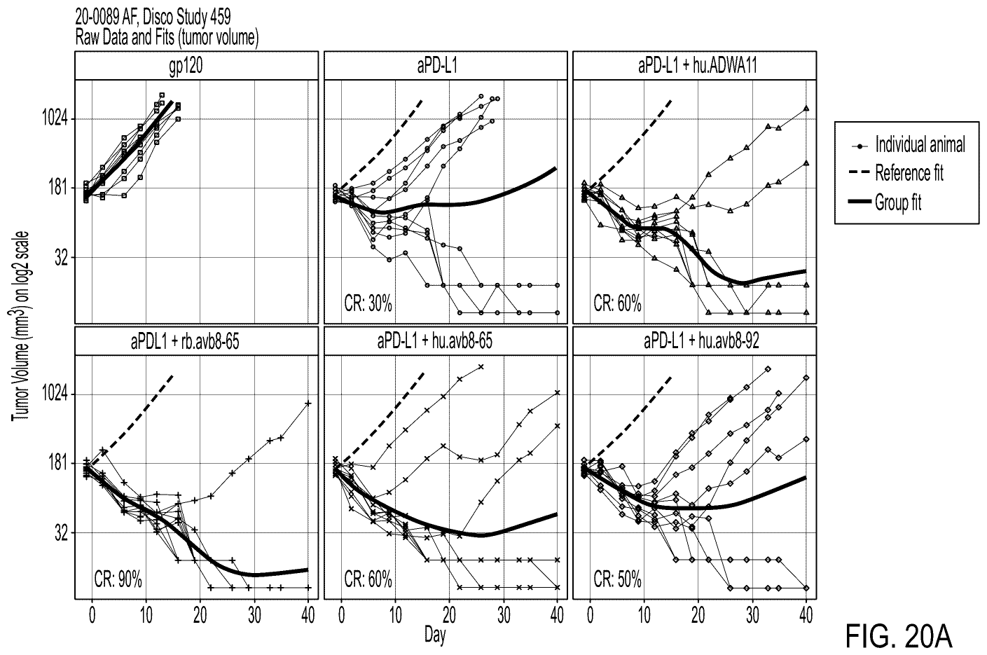

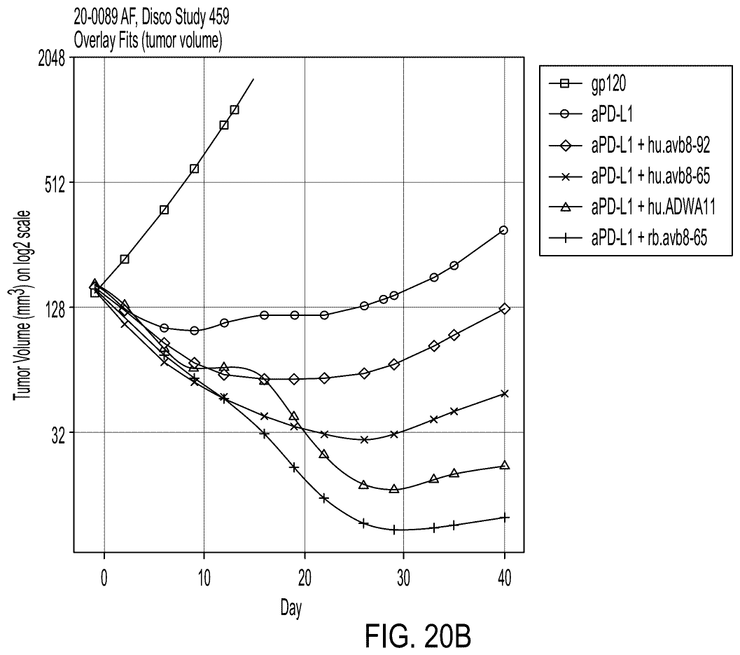

In the EMT6 model, Balb/c mice were injected with 0.1×10⁶ EMT6 cells. No significant weight loss was observed and there was CR as of Day 51 of study. Likewise, hu.avb8-65 and -92 showed tumor killing efficacy in an MC38 tumor model in combo with anti-PD-L1 (FIGS. 20A and 20B). In the MC38 model, C57BL6 mice were injected with 1×10⁶ MC38 cells. No significant weight loss was observed in mice.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Example 8: In Vivo Anti-Tumor Efficacy of αvβ8 Antibodies

The EMT6 murine mammary carcinoma cell line and female BALB/c mice were used from the same sources as in Example 7. In this Example, the following antibodies were used: (1) murine anti-gp120-IgG1 (an isotype control), (2) murine anti-gp120-IgG2a-LALAPG (an isotype control), (3) murine anti-PD-L1/IgG1 (clone 6E11; as discussed in Example 7), (4) murine Ch-anti-αvβ8 IgG2a.LALAPG (“Ch-anti-αvβ8-65”), and murine anti-αvβ8-ADWA11 IgG2a.LALAPG (see preceding Examples).

EMT6 cells were cultured in Roswell Park Memorial Institute (RPMI) 1640 medium plus 2 mM L-glutamine with 10% fetal bovine serum (FBS; HyClone, Waltham, MA). Cells in log-phase growth were centrifuged, washed once with Hank's balanced salt solution (HBSS), counted, and resuspended in 50% HBSS and 50% Matrigel (Corning; Bedford, MA) at a concentration of 1×10⁶ cells/mL for injection into mice. Mice were inoculated with 1×10⁵ cells in 100 μL of HBSS:Matrigel (1:1). EMT6 cells were inoculated in the left mammary fat pad #5 of mice. When tumor reached a volume of 130-230 mm³ animals were distributed into treatment groups based on tumor volume and treated with isotype control antibodies (murine IgG1 anti-gp120, 10 mg/kg; murine IgG2a LALAPG anti-gp120, 10 mg/kg), anti-PD-L1 (mouse IgG1 clone 6E11, 10 mg/kg), Ch-anti-αvβ8-65 (10 mg/kg), anti-αvβ8-ADWA11 (10 mg/kg), or a combination of anti-PD-L1 with Ch-anti-αvβ8-65 or anti-αvβ8-ADWA11 antibody. Antibodies were administered 2 times a week for 21 days, with the first dose intravenously, and subsequent doses intraperitoneally. Mice were euthanized if tumor volumes exceeded 1500 mm3. Animal weight change was tracked for each individual animal in the study. All animal studies were approved by a Institutional Animal Care and Use Committee.

Analyses and comparisons of tumor growth were performed using a package of customized functions in R (Version 4.1.0 (2021-05-18); R Foundation for Statistical Computing; Vienna, Austria), which integrates software from open-source packages (e.g., lme4, mgcv, gamm4, multcomp, settings, and plyr) and several packages from tidyverse (e.g., magrittr, dplyr, tidyr, and ggplot2) (Forrest et al., Generalized additive mixed modeling of longitudinal tumor growth reduces bias and improves decision making in translational oncology, Cancer Res 2020; 80(22):5089-97). Briefly, as tumors generally exhibit exponential growth, tumor volumes were subjected to natural log transformations before analysis. All raw tumor volume measurements from 0 to 8 mm3 were judged to reflect complete tumor absence and were converted to 8 mm3 prior to natural log transformation. A generalized additive mixed model was then applied to describe the changes in transformed tumor volumes over time using regression splines with automatically generated spline bases. This approach addresses both repeated measurements from the same study subjects and moderate dropouts before the end of the study. Analyses and comparisons of tumor growth were performed using a package of customized functions in R (Version: 4.1.0; R Foundation for Statistical Computing; Vienna, Austria), which integrates software from open-source packages (e.g., lme4, mgcv, gamm4, multcomp, settings, and plyr) and several packages from tidyverse (e.g., magrittr, dplyr, tidyr, and ggplot2) (Forrest et al., Generalized additive mixed modeling of longitudinal tumor growth reduces bias and improves decision making in translational oncology, Cancer Res 2020; 80(22):5089-97). Additionally, all raw tumor volume measurements less than 16 mm3 were considered too small. All raw tumor volume measurements less than 8 mm3 were judged to reflect complete tumor absence and were converted to “No Tumor Ceiling”.

Strong anti-tumor responses that resulted in reductions in tumor size were tracked as partial responses (PRs, defined as a >50% decrease from the initial tumor volume), and complete responses (CRs, defined as a 100% decrease in tumor volume). % CRs for each group of treatment were calculated using the following formula:

$$( n of CRs / n of mice in the group ) * 100$$

Tumor growth rate was calculated using the calculation of a Dunnett contrast of endpoint Gain Integrated in Time (eGaIT) estimates. Such contrast of eGaIT represents the difference in growth rates between the treatment and reference groups which are derived from the area under the curve (AUC) of the fits on the natural log scale over a common time period. To obtain the growth rate from the fit AUC on this scale in this time range, the AUC is corrected for starting tumor burden and then subjected to slope equivalence “normalization.” Mathematically, this “normalization” is attained by dividing the estimated baseline-corrected AUC value by half of the square of the common study period, resulting in units of natural log units per day. When tumors exhibit log-linear growth (i.e., the fit is a line on the natural log scale), slope equivalence “normalization” of the AUC results in the calculation of the slope of the fit. In cases where tumors demonstrate non-log-linear growth (i.e., the fit is curved on the natural log scale), slope equivalence “normalization” results in the calculation of the constant log-linear growth rate required to yield the observed baseline-corrected AUC for the fit. The more negative the contrast value, the greater the antitumor effect; the more positive the contrast value, the greater the pro-growth effect.

Comparison between anti-αvβ8 clone 65 and ADWA11 was performed by calculating: (1) the percentage of CRs of the two antibodies in combination with anti-PD-L1 (combo ADWA11 or combo clone 65) over anti-PD-L1 treatment alone in each experiment using the following formula: % CRs over anti-PD-L1=% combo CRs−anti-PDL1 CR, and (2) The percentage of mice with regressing tumors at day 14 (eGaIT <=0.05).

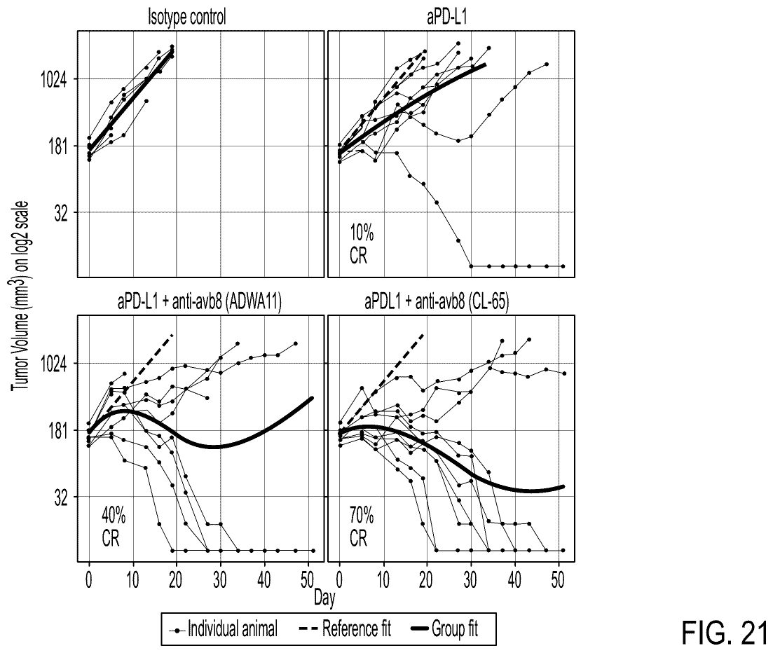

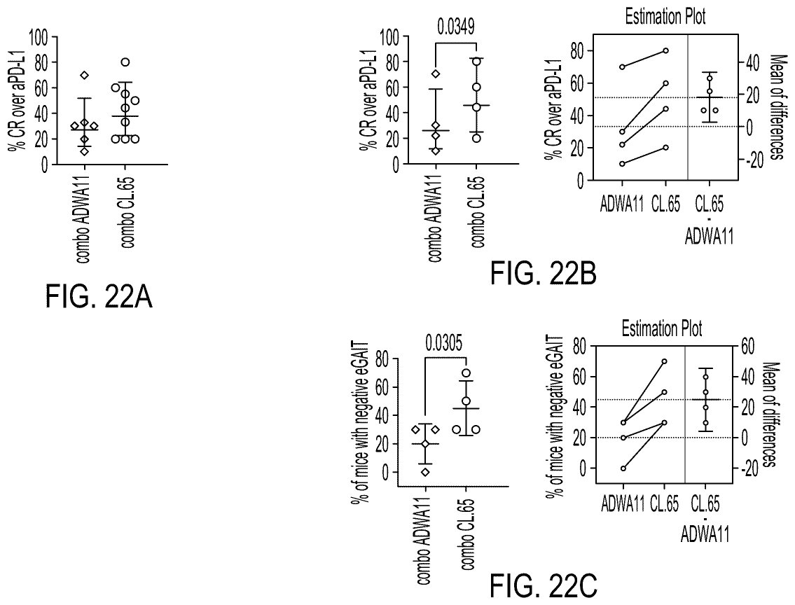

Result. FIG. 21 shows the anti-tumor activity of Ch-anti-αvβ8-65 and anti-αvβ8-ADWA11, each in combination with anti-PD-L1, compared to isotype control and anti-PD-L1 alone. Anti-PD-L1 alone did not result in a significant complete response rate, while the combination with clone 65 resulted in a significant 70% CR rate, which was significantly higher than the combination with anti-αvβ8-ADWA11. The % EGaIT at day 14 was calculated at 20% for the combination with ADWA11 and 30% for the combination with clone 65. The average eGaIT at day 14 was calculated at 0.0966 for the combination with ADWA11 and 0.0354 for the combination with clone 65. FIGS. 22A, 22B, and 22C show comparison of anti-tumor activity of Ch-anti-αvβ8-65 and anti-αvβ8-ADWA11 by (FIG. 22A) percentage of CRs in the combination groups compared to anti-PD-L1 alone across several studies, (FIG. 22B) percentage of CRs in the combination groups compared to anti-PD-L1 alone in direct comparison (i.e., the two molecules were used within the same study), and (FIG. 22C) the percentage of mice with regressing tumors at day 14, measured in studies in which both molecules were tested.

What is claimed is:

1. An antibody, or antigen-binding portion thereof, that specifically binds to αvβ8, wherein the antibody or antigen-binding portion thereof exhibits at least one of the following properties:
(a) binds to human αvβ8 with a K_{D} of 1 nM or less;
(b) binds to murine αvβ8 with a K_{D} of 1 nM or less;
(c) binds to cynomolgus αvβ8 with a K_{D} of 1 nM or less;
(d) inhibits αvβ8-mediated activation of LTGFβ 1 and LTGFβ3 presented by and/or associated with human-leucine-rich-repeat-containing protein 32 (LRRC32), LRRC32, and/or latent TGFβ-binding proteins (LTBPs); and/or
(e) blocks binding of TGFβ peptide to αvβ8.

2. The antibody, or antigen-binding portion thereof, of claim 1, comprising a light chain variable domain (VL) comprising CDR-L1, CDR-L2, and CDR-L3 and a heavy chain variable domain (VH) comprising CDR-H1, CDR-H2, and CDR-H3, wherein:
(a) CDR-L1, CDR-L2, and CDR-L2 sequences are from a VL domain of SEQ ID NO:152 and CDR-H1, CDR-H2, and CDR-H3 sequences are from a VH domain of SEQ ID NO:153;
(b) CDR-L1, CDR-L2, and CDR-L2 sequences are from a VL domain of SEQ ID NO:154 and CDR-H1, CDR-H2, and CDR-H3 sequences are from a VH domain of SEQ ID NO:155;
(c) CDR-L1, CDR-L2, and CDR-L2 sequences are from a VL domain of SEQ ID NO:156 and CDR-H1, CDR-H2, and CDR-H3 sequences are from a VH domain of SEQ ID NO:157;
(d) CDR-L1, CDR-L2, and CDR-L2 sequences are from a VL domain of SEQ ID NO:158 and CDR-H1, CDR-H2, and CDR-H3 sequences are from a VH domain of SEQ ID NO:159;
(e) CDR-L1, CDR-L2, and CDR-L2 sequences are from a VL domain of SEQ ID NO:160 and CDR-H1, CDR-H2, and CDR-H3 sequences are from a VH domain of SEQ ID NO:161;
(f) CDR-L1, CDR-L2, and CDR-L2 sequences are from a VL domain of SEQ ID NO:164 and CDR-H1, CDR-H2, and CDR-H3 sequences are from a VH domain of SEQ ID NO:165;
(g) CDR-L1, CDR-L2, and CDR-L2 sequences are from a VL domain of SEQ ID NO:166 and CDR-H1, CDR-H2, and CDR-H3 sequences are from a VH domain of SEQ ID NO:167;
(h) CDR-L1 is according to SEQ ID NO:7, CDR-L2 is according to SEQ ID NO:8, CDR-L3 is according SEQ ID NO:9, CDR-H1 is according to SEQ ID NO:10, CDR-H2 is according to SEQ ID NO:11, and CDR-H3 is according to SEQ ID NO:12;
(i) CDR-L1 is according to SEQ ID NO:13, CDR-L2 is according to SEQ ID NO:14, CDR-L3 is according SEQ ID NO:15, CDR-H1 is according to SEQ ID NO:16, CDR-H2 is according to SEQ ID NO:17, and CDR-H3 is according to SEQ ID NO:18;
(j) CDR-L1 is according to SEQ ID NO:19, CDR-L2 is according to SEQ ID NO:20, CDR-L3 is according SEQ ID NO:21, CDR-H1 is according to SEQ ID NO:22, CDR-H2 is according to SEQ ID NO:23, and CDR-H3 is according to SEQ ID NO:24;
(k) CDR-L1 is according to SEQ ID NO:25, CDR-L2 is according to SEQ ID NO:26, CDR-L3 is according SEQ ID NO:27, CDR-H1 is according to SEQ ID NO:28, CDR-H2 is according to SEQ ID NO:29, and CDR-H3 is according to SEQ ID NO:30;
(l) CDR-L1 is according to SEQ ID NO:31, CDR-L2 is according to SEQ ID NO:32, CDR-L3 is according SEQ ID NO:33, CDR-H1 is according to SEQ ID NO:34, CDR-H2 is according to SEQ ID NO:35, and CDR-H3 is according to SEQ ID NO:36;
(m) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42;
(n) CDR-L1, CDR-L2, and CDR-L2 sequences are from a VL domain of SEQ ID NO:150 and CDR-H1, CDR-H2, and CDR-H3 sequences are from a VH domain of SEQ ID NO:151;
(o) CDR-L1 is according to SEQ ID NO:1, CDR-L2 is according to SEQ ID NO:2, CDR-L3 is according SEQ ID NO:3, CDR-H1 is according to SEQ ID NO:4, CDR-H2 is according to SEQ ID NO:5, and CDR-H3 is according to SEQ ID NO:6;
(p) CDR-L1, CDR-L2, and CDR-L2 sequences are from a VL domain of SEQ ID NO:162 and CDR-H1, CDR-H2, and CDR-H3 sequences are from a VH domain of SEQ ID NO:163; or
(q) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42.

3. The antibody, or antigen-binding portion thereof, of claim 1 or claim 2, comprising a VL comprising CDR-L1, CDR-L2, and CDR-L3 and a VH comprising CDR-H1, CDR-H2, and CDR-H3, wherein CDR-L1, CDR-L2, and CDR-L2 sequences are from a VL domain of SEQ ID NO:166 and CDR-H1, CDR-H2, and CDR-H3 sequences are from a VH domain of SEQ ID NO:167.

4. The antibody, or antigen-binding portion thereof, of any one of claims 1 to 3, comprising a VL comprising CDR-L1, CDR-L2, and CDR-L3 and a VH comprising CDR-H1, CDR-H2, and CDR-H3, wherein CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42.

5. The antibody, or antigen-binding portion thereof, of claim 1 or claim 2, comprising a VL comprising CDR-L1, CDR-L2, and CDR-L3 and a VH comprising CDR-H1, CDR-H2, and CDR-H3, wherein CDR-L1, CDR-L2, and CDR-L2 sequences are from a VL domain of SEQ ID NO:154 and CDR-H1, CDR-H2, and CDR-H3 sequences are from a VH domain of SEQ ID NO:155.

6. The antibody, or antigen-binding portion thereof, of any one of claims 1, 2 and 5, comprising a VL comprising CDR-L1, CDR-L2, and CDR-L3 and a VH comprising CDR-H1, CDR-H2, and CDR-H3, wherein CDR-L1 is according to SEQ ID NO:13, CDR-L2 is according to SEQ ID NO:14, CDR-L3 is according SEQ ID NO:15, CDR-H1 is according to SEQ ID NO:16, CDR-H2 is according to SEQ ID NO:17, and CDR-H3 is according to SEQ ID NO:18.

7. An antibody, or antigen-binding portion thereof, that specifically binds to αvβ8, wherein the antibody, or antigen-binding portion thereof, comprises a heavy chain variable domain (VH) comprising CDR-H1, CDR-H2, and CDR-H3 and a light chain variable domain (VL) comprising CDR-L1, CDR-L2, and CDR-L3, wherein:
(a) CDR-L1, CDR-L2, and CDR-L2 sequences are from a VL domain of SEQ ID NO:152 and CDR-H1, CDR-H2, and CDR-H3 sequences are from a VH domain of SEQ ID NO:153;
(b) CDR-L1, CDR-L2, and CDR-L2 sequences are from a VL domain of SEQ ID NO:154 and CDR-H1, CDR-H2, and CDR-H3 sequences are from a VH domain of SEQ ID NO:155;
(c) CDR-L1, CDR-L2, and CDR-L2 sequences are from a VL domain of SEQ ID NO:156 and CDR-H1, CDR-H2, and CDR-H3 sequences are from a VH domain of SEQ ID NO:157;
(d) CDR-L1, CDR-L2, and CDR-L2 sequences are from a VL domain of SEQ ID NO:158 and CDR-H1, CDR-H2, and CDR-H3 sequences are from a VH domain of SEQ ID NO:159;
(e) CDR-L1, CDR-L2, and CDR-L2 sequences are from a VL domain of SEQ ID NO:160 and CDR-H1, CDR-H2, and CDR-H3 sequences are from a VH domain of SEQ ID NO:161;
(f) CDR-L1, CDR-L2, and CDR-L2 sequences are from a VL domain of SEQ ID NO:164 and CDR-H1, CDR-H2, and CDR-H3 sequences are from a VH domain of SEQ ID NO:165;
(g) CDR-L1, CDR-L2, and CDR-L2 sequences are from a VL domain of SEQ ID NO:166 and CDR-H1, CDR-H2, and CDR-H3 sequences are from a VH domain of SEQ ID NO:167;
(h) CDR-L1 is according to SEQ ID NO:7, CDR-L2 is according to SEQ ID NO:8, CDR-L3 is according SEQ ID NO:9, CDR-H1 is according to SEQ ID NO:10, CDR-H2 is according to SEQ ID NO:11, and CDR-H3 is according to SEQ ID NO:12;
(i) CDR-L1 is according to SEQ ID NO:13, CDR-L2 is according to SEQ ID NO:14, CDR-L3 is according SEQ ID NO:15, CDR-H1 is according to SEQ ID NO:16, CDR-H2 is according to SEQ ID NO:17, and CDR-H3 is according to SEQ ID NO:18;
(j) CDR-L1 is according to SEQ ID NO:19, CDR-L2 is according to SEQ ID NO:20, CDR-L3 is according SEQ ID NO:21, CDR-H1 is according to SEQ ID NO:22, CDR-H2 is according to SEQ ID NO:23, and CDR-H3 is according to SEQ ID NO:24;
(k) CDR-L1 is according to SEQ ID NO:25, CDR-L2 is according to SEQ ID NO:26, CDR-L3 is according SEQ ID NO:27, CDR-H1 is according to SEQ ID NO:28, CDR-H2 is according to SEQ ID NO:29, and CDR-H3 is according to SEQ ID NO:30;
(l) CDR-L1 is according to SEQ ID NO:31, CDR-L2 is according to SEQ ID NO:32, CDR-L3 is according SEQ ID NO:33, CDR-H1 is according to SEQ ID NO:34, CDR-H2 is according to SEQ ID NO:35, and CDR-H3 is according to SEQ ID NO:36;
(m) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42;
(n) CDR-L1, CDR-L2, and CDR-L2 sequences are from a VL domain of SEQ ID NO:150 and CDR-H1, CDR-H2, and CDR-H3 sequences are from a VH domain of SEQ ID NO:151;
(o) CDR-L1 is according to SEQ ID NO:1, CDR-L2 is according to SEQ ID NO:2, CDR-L3 is according SEQ ID NO:3, CDR-H1 is according to SEQ ID NO:4, CDR-H2 is according to SEQ ID NO:5, and CDR-H3 is according to SEQ ID NO:6;
(p) CDR-L1, CDR-L2, and CDR-L2 sequences are from a VL domain of SEQ ID NO:162 and CDR-H1, CDR-H2, and CDR-H3 sequences are from a VH domain of SEQ ID NO:163; or
(q) CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42.

8. An antibody, or antigen-binding portion thereof, that specifically binds to αvβ8, wherein the antibody, or antigen-binding portion thereof, comprises a VH comprising CDR-H1, CDR-H2, and CDR-H3 and a VL comprising CDR-L1, CDR-L2, and CDR-L3, wherein CDR-L1, CDR-L2, and CDR-L2 sequences are from a VL domain of SEQ ID NO:166 and CDR-H1, CDR-H2, and CDR-H3 sequences are from a VH domain of SEQ ID NO:167.

9. An antibody, or antigen-binding portion thereof, that specifically binds to αvβ8, wherein the antibody, or antigen-binding portion thereof, comprises a VH comprising CDR-H1, CDR-H2, and CDR-H3 and a VL comprising CDR-L1, CDR-L2, and CDR-L3, wherein CDR-L1 is according to SEQ ID NO:37, CDR-L2 is according to SEQ ID NO:38, CDR-L3 is according SEQ ID NO:39, CDR-H1 is according to SEQ ID NO:40, CDR-H2 is according to SEQ ID NO:41, and CDR-H3 is according to SEQ ID NO:42.

10. An antibody, or antigen-binding portion thereof, that specifically binds to αvβ8, wherein the antibody, or antigen-binding portion thereof, comprises a VH comprising CDR-H1, CDR-H2, and CDR-H3 and a VL comprising CDR-L1, CDR-L2, and CDR-L3, wherein CDR-L1, CDR-L2, and CDR-L2 sequences are from a VL domain of SEQ ID NO:154 and CDR-H1, CDR-H2, and CDR-H3 sequences are from a VH domain of SEQ ID NO:155.

11. An antibody, or antigen-binding portion thereof, that specifically binds to αvβ8, wherein the antibody, or antigen-binding portion thereof, comprises a VH comprising CDR-H1, CDR-H2, and CDR-H3 and a VL comprising CDR-L1, CDR-L2, and CDR-L3, wherein CDR-L1 is according to SEQ ID NO:13, CDR-L2 is according to SEQ ID NO:14, CDR-L3 is according SEQ ID NO:15, CDR-H1 is according to SEQ ID NO:16, CDR-H2 is according to SEQ ID NO:17, and CDR-H3 is according to SEQ ID NO:18;

12. The antibody, or antigen-binding portion thereof, of any one of claims 1 to 11, which is a monoclonal antibody.

13. The antibody, or antigen-binding portion thereof, of any one of claims 1 to 12, which is a humanized or chimeric antibody.

14. The antibody, or antigen-binding portion thereof, of any one of claims 1 to 13, which is an antibody fragment that specifically binds human αvβ8.

15. The antibody, or antigen-binding portion thereof, of any one of claims 1 to 14, comprising a sequence selected from the group consisting of
(a) a VL sequence having at least 95% sequence identity to the amino acid sequence of SEQ ID NO:152 and a VH sequence having at least 95% sequence identity to the amino acid sequence of SEQ ID NO:153;
(b) a VL sequence having at least 95% sequence identity to the amino acid sequence of SEQ ID NO:154 and a VH sequence having at least 95% sequence identity to the amino acid sequence of SEQ ID NO:155;
(c) a VL sequence having at least 95% sequence identity to the amino acid sequence of SEQ ID NO:156 and a VH sequence having at least 95% sequence identity to the amino acid sequence of SEQ ID NO:157;
(d) a VL sequence having at least 95% sequence identity to the amino acid sequence of SEQ ID NO:158 and a VH sequence having at least 95% sequence identity to the amino acid sequence of SEQ ID NO:159;
(e) a VL sequence having at least 95% sequence identity to the amino acid sequence of SEQ ID NO:160 and a VH sequence having at least 95% sequence identity to the amino acid sequence of SEQ ID NO:161;
(f) a VL sequence having at least 95% sequence identity to the amino acid sequence of SEQ ID NO:164 and a VH sequence having at least 95% sequence identity to the amino acid sequence of SEQ ID NO:165;
(g) a VL sequence having at least 95% sequence identity to the amino acid sequence of SEQ ID NO:166 and a VH sequence having at least 95% sequence identity to the amino acid sequence of SEQ ID NO:167; and
(h) a VL sequence having at least 95% sequence identity to the amino acid sequence of SEQ ID NO:162 and a VH sequence having at least 95% sequence identity to the amino acid sequence of SEQ ID NO:163.

16. The antibody, or antigen-binding portion thereof, of any one of claims 1-4, 7-9, and 12-15, comprising a VL sequence having at least 95% sequence identity to the amino acid sequence of SEQ ID NO:166 and a VH sequence having at least 95% sequence identity to the amino acid sequence of SEQ ID NO:167.

17. The antibody, or antigen-binding portion thereof, of any one of claims 1-2, 5-7, and 10-15, comprising a VL sequence having at least 95% sequence identity to the amino acid sequence of SEQ ID NO:154 and a VH sequence having at least 95% sequence identity to the amino acid sequence of SEQ ID NO:155.

18. The antibody, or antigen-binding portion thereof, of any one of claims 1 to 17, comprising a sequence selected from the group consisting of:
(a) a VL sequence comprising the sequence of amino acids set forth in SEQ ID NO:152 and a VH sequence comprising the sequence of amino acids set forth in SEQ ID NO:153;
(b) a VL sequence comprising the sequence of amino acids set forth in SEQ ID NO:154 and a VH sequence comprising the sequence of amino acids set forth in SEQ ID NO:155;
(c) a VL sequence comprising the sequence of amino acids set forth in SEQ ID NO:156 and a VH sequence comprising the sequence of amino acids set forth in SEQ ID NO:157;
(d) a VL sequence comprising the sequence of amino acids set forth in SEQ ID NO:158 and a VH sequence comprising the sequence of amino acids set forth in SEQ ID NO:159;
(e) a VL sequence comprising the sequence of amino acids set forth in SEQ ID NO:160 and a VH sequence comprising the sequence of amino acids set forth in SEQ ID NO:161;
(f) a VL sequence comprising the sequence of amino acids set forth in SEQ ID NO:164 and a VH sequence comprising the sequence of amino acids set forth in SEQ ID NO:165;
(g) a VL sequence comprising the sequence of amino acids set forth in SEQ ID NO:166 and a VH sequence comprising the sequence of amino acids set forth in SEQ ID NO:167;
(h) a VL sequence comprising the sequence of amino acids set forth in SEQ ID NO:150 and a VH sequence comprising the sequence of amino acids set forth in SEQ ID NO:151; and
(i) a VL sequence comprising the sequence of amino acids set forth in SEQ ID NO:162 and a VH sequence comprising the sequence of amino acids set forth in SEQ ID NO:163.

19. The antibody, or antigen-binding portion thereof, of any one of claims 1-4, 7-9, 12-15, 16, and 18, comprising a VL sequence comprising the sequence of amino acids set forth in SEQ ID NO:166 and a VH sequence comprising the sequence of amino acids set forth in SEQ ID NO:167.

20. The antibody, or antigen-binding portion thereof, of any one of claims 1-2, 5-7, 10-15, 17, and 18, comprising a VL sequence comprising the sequence of amino acids set forth in SEQ ID NO:154 and a VH sequence comprising the sequence of amino acids set forth in SEQ ID NO:155.

21. The antibody, or antigen-binding portion thereof, of any one of claims 1 to 20, which is a full-length antibody of IgG1 isotype.

22. The antibody, or antigen-binding portion thereof, of any one of claims 1 to 21, comprising a variant IgG1 Fc region with reduced effector function.

23. The antibody, or antigen-binding portion thereof, of claim 21 or claim 22 wherein the Fc region comprises amino acid substitutions L234A/L235A with numbering according to the EU index of Kabat.

24. The antibody, or antigen binding portion thereof, of any one of claims 21 to 23 wherein the Fc region comprises amino acid substitution P329G with numbering according to the EU index of Kabat.

25. The antibody, or antigen-binding portion thereof, of any one of claims 1 to 24, wherein the antibody binds human αvβ8 with a KD of 1 nM or less as measured by surface plasmon resonance.

26. The antibody, or antigen-binding portion thereof, of any one of claims 1 to 25 comprising:
(a) a heavy chain exhibiting at least 95% sequence identity to the sequence of amino acids set forth in SEQ ID NO:201 and a light chain exhibiting at least 95% sequence identity to the sequence of amino acids set forth in SEQ ID NO:200;
(b) a heavy chain exhibiting at least 95% sequence identity to the sequence of amino acids set forth in SEQ ID NO:203 and a light chain exhibiting at least 95% sequence identity to the sequence of amino acids set forth in SEQ ID NO:202;
(c) a heavy chain exhibiting at least 95% sequence identity to the sequence of amino acids set forth in SEQ ID NO:220 and a light chain exhibiting at least 95% sequence identity to the sequence of amino acids set forth in SEQ ID NO:200; or
(d) a heavy chain exhibiting at least 95% sequence identity to the sequence of amino acids set forth in SEQ ID NO:221 and a light chain exhibiting at least 95% sequence identity to the sequence of amino acids set forth in SEQ ID NO:202.

27. The antibody, or antigen-binding portion thereof, of any one of claims 1-4, 7-9, 12-15, 16, 18, 19, and 21-26, comprising a heavy chain exhibiting at least 95% sequence identity to the sequence of amino acids set forth in SEQ ID NO:203 and a light chain exhibiting at least 95% sequence identity to the sequence of amino acids set forth in SEQ ID NO:202.

28. The antibody, or antigen-binding portion thereof, of any one of claims 1-2, 5-7, 10-15, 17, 18, and 20-26, comprising a heavy chain exhibiting at least 95% sequence identity to the sequence of amino acids set forth in SEQ ID NO:201 and a light chain exhibiting at least 95% sequence identity to the sequence of amino acids set forth in SEQ ID NO:200.

29. The antibody, or antigen-binding portion thereof, of any one of claims 1 to 28 comprising:
(a) a heavy chain of SEQ ID NO:201 and a light chain of SEQ ID NO:200;
(b) a heavy chain of SEQ ID NO:203 and a light chain of SEQ ID NO:202;
(c) a heavy chain of SEQ ID NO:220 and a light chain of SEQ ID NO:200; or
(d) a heavy chain of SEQ ID NO:221 and a light chain of SEQ ID NO:202.

30. The antibody, or antigen-binding portion thereof, of any one of claims 1-4, 7-9, 12-15, 16, 18, 19, 21-26 and 29, comprising a heavy chain of SEQ ID NO:203 and a light chain of SEQ ID NO:202 or a heavy chain of SEQ ID NO:221 and a light chain of SEQ ID NO:202.

31. The antibody, or antigen-binding portion thereof, of any one of claims 1-2, 5-7, 10-15, 17, 18, 20-26 and 29, comprising a heavy chain of SEQ ID NO:201 and a light chain of SEQ ID NO:200 or a heavy chain of SEQ ID NO:220 and a light chain of SEQ ID NO:200.

32. An isolated nucleic acid encoding the antibody of any one of claims 1 to 31.

33. A vector comprising the nucleic acid of claim 32.

34. A host cell comprising the nucleic acid of claim 32 or the vector of claim 33.

35. A method of producing an antibody that binds to αvβ8 comprising culturing the host cell of claim 34 under conditions suitable for the expression of the antibody.

36. The method of claim 35, further comprising recovering the antibody from the host cell.

37. An antibody produced by the method of claim 36.

38. A pharmaceutical composition comprising the antibody or antigen-binding portion thereof of any one of claims 1 to 31 and a pharmaceutically acceptable carrier.

39. A method of treating cancer in an individual in need thereof, the method comprising administering to the individual an effective amount of the antibody, or antigen-binding portion thereof, of any one of claims 1 to 31 or the pharmaceutical composition of claim 38.

40. A method of treating cancer in an individual in need thereof, the method comprising administering:
(a) an effective amount of the antibody or antigen-binding fragment thereof of any one of claims 1 to 31 or the pharmaceutical composition of claim 38, and
(b) an effective amount of a PD-1 axis antagonist.

41. The method of claim 40, wherein the PD-1 axis antagonist is an anti-PD-L1 antibody.

42. The method of claim 40 or claim 41, wherein the PD-1 axis antagonist is atezolizumab.

43. The method of any one of claims 39 to 42, further comprising assessing the level of αvβ6 expressed in the cancer.

44. The method of any one of claims 39 to 43, wherein the cancer is selected from the list of ovarian cancer, triple-negative breast cancer, non-small cell lung cancer, colorectal cancer, cholangiocarcinoma, endometrial cancer, kidney renal papillary cancer, and bladder cancer.


SEQUENCE LISTING (221 sequences)

SEQ ID NO: 1 - protein, 11 aa, synthetic construct
QASENINSLLA

SEQ ID NO: 2 - protein, 7 aa, synthetic construct
RASTLAS

SEQ ID NO: 3 - protein, 15 aa, synthetic construct
QSNYGFTGSGAFFYG

SEQ ID NO: 4 - protein, 5 aa, synthetic construct
NYAMI

SEQ ID NO: 5 - protein, 16 aa, synthetic construct
IISSSDRIWYASWVKG

SEQ ID NO: 6 - protein, 12 aa, synthetic construct
SVSNSYFDGWNL

SEQ ID NO: 7 - protein, 11 aa, synthetic construct
QASENINSLLA

SEQ ID NO: 8 - protein, 7 aa, synthetic construct
RASTLAS

SEQ ID NO: 9 - protein, 15 aa, synthetic construct
QSNYGFTGSGAFFYG

SEQ ID NO: 10 - protein, 5 aa, synthetic construct
NYAMI

SEQ ID NO: 11 - protein, 16 aa, synthetic construct
IISSSDRIWYASWVKG

SEQ ID NO: 12 - protein, 12 aa, synthetic construct
SVSNSYFDGWNL

SEQ ID NO: 13 - protein, 11 aa, synthetic construct
QASENINSLLA

SEQ ID NO: 14 - protein, 7 aa, synthetic construct
RASTLAS

SEQ ID NO: 15 - protein, 15 aa, synthetic construct
QSNYGFTGSGAFFYG

SEQ ID NO: 16 - protein, 5 aa, synthetic construct
NYAMI

SEQ ID NO: 17 - protein, 16 aa, synthetic construct
IISSSDRIWYASWVKG

SEQ ID NO: 18 - protein, 12 aa, synthetic construct
SVSNSYFDGWNL

SEQ ID NO: 19 - protein, 11 aa, synthetic construct
QASENIQSLLA

SEQ ID NO: 20 - protein, 7 aa, synthetic construct
RASTLAS

SEQ ID NO: 21 - protein, 15 aa, synthetic construct
QSNYGFTGSGAFFYG

SEQ ID NO: 22 - protein, 5 aa, synthetic construct
NYAMI

SEQ ID NO: 23 - protein, 16 aa, synthetic construct
IISSSDRIWYASWVKG

SEQ ID NO: 24 - protein, 12 aa, synthetic construct
SVSNSYFDGWNL

SEQ ID NO: 25 - protein, 11 aa, synthetic construct
QASENINALLA

SEQ ID NO: 26 - protein, 7 aa, synthetic construct
RASTLAS

SEQ ID NO: 27 - protein, 15 aa, synthetic construct
QSNYGFTGSGAFFYG

SEQ ID NO: 28 - protein, 5 aa, synthetic construct
NYAMI

SEQ ID NO: 29 - protein, 16 aa, synthetic construct
IISSSDRIWYASWVKG

SEQ ID NO: 30 - protein, 12 aa, synthetic construct
SVSNSYFDGWNL

SEQ ID NO: 31 - protein, 11 aa, synthetic construct
QASENINTLLA

SEQ ID NO: 32 - protein, 7 aa, synthetic construct
RASTLAS

SEQ ID NO: 33 - protein, 15 aa, synthetic construct
QSNYGFTGSGAFFYG

SEQ ID NO: 34 - protein, 5 aa, synthetic construct
NYAMI

SEQ ID NO: 35 - protein, 16 aa, synthetic construct
IISSSDRIWYASWVKG

SEQ ID NO: 36 - protein, 12 aa, synthetic construct
SVSNSYFDGWNL

SEQ ID NO: 37 - protein, 13 aa, synthetic construct
QSIKSVGDNKWLA

SEQ ID NO: 38 - protein, 7 aa, synthetic construct
DASDLAS

SEQ ID NO: 39 - protein, 10 aa, synthetic construct
AGSVSSGDSG

SEQ ID NO: 40 - protein, 5 aa, synthetic construct
SYAMG

SEQ ID NO: 41 - protein, 16 aa, synthetic construct
VIRKDDNTYYGNWAKG

SEQ ID NO: 42 - protein, 13 aa, synthetic construct
ALNTYVGFPYFNI

SEQ ID NO: 43 - protein, 11 aa, synthetic construct
QASENINSLLA

SEQ ID NO: 44 - protein, 7 aa, synthetic construct
RASTLAS

SEQ ID NO: 45 - protein, 15 aa, synthetic construct
QSNYGFTGSGAFFYG

SEQ ID NO: 46 - protein, 7 aa, synthetic construct
GIDLSNY

SEQ ID NO: 47 - protein, 5 aa, synthetic construct
SSSDR

SEQ ID NO: 48 - protein, 12 aa, synthetic construct
SVSNSYFDGWNL

SEQ ID NO: 49 - protein, 11 aa, synthetic construct
QASENINSLLA

SEQ ID NO: 50 - protein, 7 aa, synthetic construct
RASTLAS

SEQ ID NO: 51 - protein, 15 aa, synthetic construct
QSNYGFTGSGAFFYG

SEQ ID NO: 52 - protein, 7 aa, synthetic construct
GIDLSNY

SEQ ID NO: 53 - protein, 5 aa, synthetic construct
SSSDR

SEQ ID NO: 54 - protein, 12 aa, synthetic construct
SVSNSYFDGWNL

SEQ ID NO: 55 - protein, 11 aa, synthetic construct
QASENINSLLA

SEQ ID NO: 56 - protein, 7 aa, synthetic construct
RASTLAS

SEQ ID NO: 57 - protein, 15 aa, synthetic construct
QSNYGFTGSGAFFYG

SEQ ID NO: 58 - protein, 7 aa, synthetic construct
GIDLSNY

SEQ ID NO: 59 - protein, 5 aa, synthetic construct
SSSDR

SEQ ID NO: 60 - protein, 12 aa, synthetic construct
SVSNSYFDGWNL

SEQ ID NO: 61 - protein, 11 aa, synthetic construct
QASENINSLLA

SEQ ID NO: 62 - protein, 7 aa, synthetic construct
RASTLAS

SEQ ID NO: 63 - protein, 15 aa, synthetic construct
QSNYGFTGSGAFFYG

SEQ ID NO: 64 - protein, 7 aa, synthetic construct
GIDLSNY

SEQ ID NO: 65 - protein, 5 aa, synthetic construct
SSSDR

SEQ ID NO: 66 - protein, 12 aa, synthetic construct
SVSNSYFDGWNL

SEQ ID NO: 67 - protein, 11 aa, synthetic construct
QASENINSLLA

SEQ ID NO: 68 - protein, 7 aa, synthetic construct
RASTLAS

SEQ ID NO: 69 - protein, 15 aa, synthetic construct
QSNYGFTGSGAFFYG

SEQ ID NO: 70 - protein, 7 aa, synthetic construct
GIDLSNY

SEQ ID NO: 71 - protein, 5 aa, synthetic construct
SSSDR

SEQ ID NO: 72 - protein, 12 aa, synthetic construct
SVSNSYFDGWNL

SEQ ID NO: 73 - protein, 11 aa, synthetic construct
QASENINSLLA

SEQ ID NO: 74 - protein, 7 aa, synthetic construct
RASTLAS

SEQ ID NO: 75 - protein, 15 aa, synthetic construct
QSNYGFTGSGAFFYG

SEQ ID NO: 76 - protein, 7 aa, synthetic construct
GIDLSNY

SEQ ID NO: 77 - protein, 5 aa, synthetic construct
SSSDR

SEQ ID NO: 78 - protein, 12 aa, synthetic construct
SVSNSYFDGWNL

SEQ ID NO: 79 - protein, 13 aa, synthetic construct
QSIKSVGDNKWLA

SEQ ID NO: 80 - protein, 7 aa, synthetic construct
DASDLAS

SEQ ID NO: 81 - protein, 10 aa, synthetic construct
AGSVSSGDSG

SEQ ID NO: 82 - protein, 7 aa, synthetic construct
GFSLSSY

SEQ ID NO: 83 - protein, 5 aa, synthetic construct
RKDDN

SEQ ID NO: 84 - protein, 13 aa, synthetic construct
ALNTYVGFPYFNI

SEQ ID NO: 85 - protein, 7 aa, synthetic construct
NSLLAWY

SEQ ID NO: 86 - protein, 10 aa, synthetic construct
LLIYRASTLA

SEQ ID NO: 87 - protein, 14 aa, synthetic construct
QSNYGFTGSGAFFY

SEQ ID NO: 88 - protein, 6 aa, synthetic construct
SNYAMI

SEQ ID NO: 89 - protein, 12 aa, synthetic construct
WIGIISSSDRIW

SEQ ID NO: 90 - protein, 12 aa, synthetic construct
SRSVSNSYFDGW

SEQ ID NO: 91 - protein, 7 aa, synthetic construct
NSLLAWY

SEQ ID NO: 92 - protein, 10 aa, synthetic construct
LLIYRASTLA

SEQ ID NO: 93 - protein, 14 aa, synthetic construct
QSNYGFTGSGAFFY

SEQ ID NO: 94 - protein, 6 aa, synthetic construct
SNYAMI

SEQ ID NO: 95 - protein, 12 aa, synthetic construct
WIGIISSSDRIW

SEQ ID NO: 96 - protein, 12 aa, synthetic construct
SRSVSNSYFDGW

SEQ ID NO: 97 - protein, 7 aa, synthetic construct
NSLLAWY

SEQ ID NO: 98 - protein, 10 aa, synthetic construct
LLIYRASTLA

SEQ ID NO: 99 - protein, 14 aa, synthetic construct
QSNYGFTGSGAFFY

SEQ ID NO: 100 - protein, 6 aa, synthetic construct
SNYAMI

SEQ ID NO: 101 - protein, 12 aa, synthetic construct
WIGIISSSDRIW

SEQ ID NO: 102 - protein, 12 aa, synthetic construct
SRSVSNSYFDGW

SEQ ID NO: 103 - protein, 7 aa, synthetic construct
NSLLAWY

SEQ ID NO: 104 - protein, 10 aa, synthetic construct
LLIYRASTLA

SEQ ID NO: 105 - protein, 14 aa, synthetic construct
QSNYGFTGSGAFFY

SEQ ID NO: 106 - protein, 6 aa, synthetic construct
SNYAMI

SEQ ID NO: 107 - protein, 12 aa, synthetic construct
WIGIISSSDRIW

SEQ ID NO: 108 - protein, 12 aa, synthetic construct
SRSVSNSYFDGW

SEQ ID NO: 109 - protein, 7 aa, synthetic construct
NSLLAWY

SEQ ID NO: 110 - protein, 10 aa, synthetic construct
LLIYRASTLA

SEQ ID NO: 111 - protein, 14 aa, synthetic construct
QSNYGFTGSGAFFY

SEQ ID NO: 112 - protein, 6 aa, synthetic construct
SNYAMI

SEQ ID NO: 113 - protein, 12 aa, synthetic construct
WIGIISSSDRIW

SEQ ID NO: 114 - protein, 12 aa, synthetic construct
SRSVSNSYFDGW

SEQ ID NO: 115 - protein, 7 aa, synthetic construct
NSLLAWY

SEQ ID NO: 116 - protein, 10 aa, synthetic construct
LLIYRASTLA

SEQ ID NO: 117 - protein, 14 aa, synthetic construct
QSNYGFTGSGAFFY

SEQ ID NO: 118 - protein, 6 aa, synthetic construct
SNYAMI

SEQ ID NO: 119 - protein, 12 aa, synthetic construct
WIGIISSSDRIW

SEQ ID NO: 120 - protein, 12 aa, synthetic construct
SRSVSNSYFDGW

SEQ ID NO: 121 - protein, 7 aa, synthetic construct
NKWLAWY

SEQ ID NO: 122 - protein, 10 aa, synthetic construct
LLIYDASDLA

SEQ ID NO: 123 - protein, 9 aa, synthetic construct
AGSVSSGDS

SEQ ID NO: 124 - protein, 6 aa, synthetic construct
SSYAMG

SEQ ID NO: 125 - protein, 12 aa, synthetic construct
WIGVIRKDDNTY

SEQ ID NO: 126 - protein, 12 aa, synthetic construct
ARALNTYVGFPY

SEQ ID NO: 127-149 - skipped (23 entries)

SEQ ID NO: 150 - protein, 113 aa, synthetic construct
DIVMTQTPASVEAAVGGTVTIKCQASENINSLLAWYQQKPGQPPKLLIYRASTLASGVPSRFRGSGSGTQFTLTISDLECDDAATYYCQSNYGFTGSGAFFYGFGGGTEVVVE

SEQ ID NO: 151 - protein, 118 aa, synthetic construct
QSVEESGGRLVTPGTPLTLTCTVSGIDLSNYAMIWVRQAPGKGLEWIGIISSSDRIWYASWVKGRFTISKTSSTTVDLQMTSLTTEDTATYFCSRSVSNSYFDGWNLWGPGTLVTVSS

SEQ ID NO: 152 - protein, 113 aa, synthetic construct
DIQMTQSPSSVSASVGDRVTITCQASENINSLLAWYQQKPGKPPKLLIYRASTLASGVPSRFSGSGSGTDFTLTISSLQPEDFATYYCQSNYGFTGSGAFFYGFGQGTKVEIK

SEQ ID NO: 153 - protein, 120 aa, synthetic construct
EQQLLESGGGLVQPGGSLRLSCAVSGIDLSNYAMIWVRQAPGKGLEWIGIISSSDRIWYASWVKGRFTISKDSSKTTVYLQMNSLRAEDTAVYFCSRSVSNSYFDGWNLWGPGTLVTVSS

SEQ ID NO: 154 - protein, 113 aa, synthetic construct
DIQMTQSPSSVSASVGDRVTITCQASENINSLLAWYQQKPGKAPKLLIYRASTLASGVPSRFSGSGSGTDFTLTISSLQPEDFATYYCQSNYGFTGSGAFFYGFGQGTKVEIK

SEQ ID NO: 155 - protein, 120 aa, synthetic construct
EVQLLESGGGLVQPGGSLRLSCAASGIDLSNYAMIWVRQAPGKGLEWVGIISSSDRIWYASWVKGRFTISRDNSKNTLYLQMNSLRAEDTAVYYCSRSVSNSYFDGWNLWGQGTLVTVSS

SEQ ID NO: 156 - protein, 113 aa, synthetic construct
DIQMTQSPSSVSASVGDRVTITCQASENIQSLLAWYQQKPGKAPKLLIYRASTLASGVPSRFSGSGSGTDFTLTISSLQPEDFATYYCQSNYGFTGSGAFFYGFGQGTKVEIK

SEQ ID NO: 157 - protein, 120 aa, synthetic construct; identical to SEQ ID NO: 155

SEQ ID NO: 158 - protein, 113 aa, synthetic construct
DIQMTQSPSSVSASVGDRVTITCQASENINALLAWYQQKPGKAPKLLIYRASTLASGVPSRFSGSGSGTDFTLTISSLQPEDFATYYCQSNYGFTGSGAFFYGFGQGTKVEIK

SEQ ID NO: 159 - protein, 120 aa, synthetic construct; identical to SEQ ID NO: 155

SEQ ID NO: 160 - protein, 113 aa, synthetic construct
DIQMTQSPSSVSASVGDRVTITCQASENINTLLAWYQQKPGKAPKLLIYRASTLASGVPSRFSGSGSGTDFTLTISSLQPEDFATYYCQSNYGFTGSGAFFYGFGQGTKVEIK

SEQ ID NO: 161 - protein, 120 aa, synthetic construct; identical to SEQ ID NO: 155

SEQ ID NO: 162 - protein, 110 aa, synthetic construct
AAVLTQTPSPVSAAVGGTVSISCQSIKSVGDNKWLAWYQQKPGQPPKLLIYDASDLASGVPSRFKGSGSRTQFTLTISDLQCDDAATYYCAGSVSSGDSGFGGGTEVVVA

SEQ ID NO: 163 - protein, 118 aa, synthetic construct
QSVEESGGRLVTPGTPLTLTCTVSGFSLSSYAMGWVRQAPGKGLEWIGVIRKDDNTYYGNWAKGRFTISRTSTTVDLKMTSLTAADTATYFCARALNTYVGFPYFNIWGPGTLVTVSS

SEQ ID NO: 164 - protein, 110 aa, synthetic construct
DAVLTQSPDSLAVSLGERATINCQSIKSVGDNKWLAWYQQKPGQPPKLLIYDASDLASGVPDRFSGSGSRTDFTLTISSLQAEDVAVYYCAGSVSSGDSGFGGGTKVEIK

SEQ ID NO: 165 - protein, 121 aa, synthetic construct
EQQLLESGGGLVQPGGSLRLSCAVSGFSLSSYAMGWVRQAPGKGLEWIGVIRKDDNTYYGNWAKGRFTISRDSSKNTVYLQMNSLRAEDTAVYFCARALNTYVGFPYFNIWGPGTLVTVSS

SEQ ID NO: 166 - protein, 110 aa, synthetic construct; identical to SEQ ID NO: 164

SEQ ID NO: 167 - protein, 121 aa, synthetic construct
EQQLLESGGGLVQPGGSLRLSCAASGFSLSSYAMGWVRQAPGKGLEWIGVIRKDDNTYYGNWAKGRFTISRDSSKNTLYLQMNSLRAEDTAVYFCARALNTYVGFPYFNIWGPGTLVTVSS

SEQ ID NO: 168-199 - skipped (32 entries)

SEQ ID NO: 200 - protein, 220 aa, synthetic construct
DIQMTQSPSSVSASVGDRVTITCQASENINSLLAWYQQKPGKAPKLLIYRASTLASGVPSRFSGSGSGTDFTLTISSLQPEDFATYYCQSNYGFTGSGAFFYGFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 450 aa, synthetic construct
EVQLLESGGGLVQPGGSLRLSCAASGIDLSNYAMIWVRQAPGKGLEWVGIISSSDRIWYASWVKGRFTISRDNSKNTLYLQMNSLRAEDTAVYYCSRSVSNSYFDGWNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 217 aa, synthetic construct
DAVLTQSPDSLAVSLGERATINCQSIKSVGDNKWLAWYQQKPGQPPKLLIYDASDLASGVPDRFSGSGSRTDFTLTISSLQAEDVAVYYCAGSVSSGDSGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 451 aa, synthetic construct
EQQLLESGGGLVQPGGSLRLSCAASGFSLSSYAMGWVRQAPGKGLEWIGVIRKDDNTYYGNWAKGRFTISRDSSKNTLYLQMNSLRAEDTAVYFCARALNTYVGFPYFNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205-209 - skipped (5 entries)

SEQ ID NO: 210 - protein, 10 aa, synthetic construct
GFTFSDSWIH

SEQ ID NO: 211 - protein, 18 aa, synthetic construct
AWISPYGGSTYYADSVKG

SEQ ID NO: 212 - protein, 9 aa, synthetic construct
RHWPGGFDY

SEQ ID NO: 213 - protein, 11 aa, synthetic construct
RASQDVSTAVA

SEQ ID NO: 214 - protein, 7 aa, synthetic construct
SASFLYS

SEQ ID NO: 215 - protein, 9 aa, synthetic construct
QQYLYHPAT

SEQ ID NO: 216 - protein, 118 aa, synthetic construct [sequence omitted]

SEQ ID NO: 217 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 218 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 449 aa, synthetic construct
EVQLLESGGGLVQPGGSLRLSCAASGIDLSNYAMIWVRQAPGKGLEWVGIISSSDRIWYASWVKGRFTISRDNSKNTLYLQMNSLRAEDTAVYYCSRSVSNSYFDGWNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 221 - protein, 450 aa, synthetic construct
EQQLLESGGGLVQPGGSLRLSCAASGFSLSSYAMGWVRQAPGKGLEWIGVIRKDDNTYYGNWAKGRFTISRDSSKNTLYLQMNSLRAEDTAVYFCARALNTYVGFPYFNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG